(19) United States
(12) Patent Application Publication
(10) Pub. No.: US 2022/0211489 A1
(43) Pub. Date: Jul. 7, 2022

(54) METHODS AND DEVICES FOR WAVEFRONT TREATMENTS OF ASTIGMATISM, COMA, PRESBYOPIA IN HUMAN EYES

(71) Applicants: Junzhong Liang, Freemont, CA (US); Ling Yu, Freemont, CA (US)

(72) Inventors: Junzhong Liang, Freemont, CA (US); Ling Yu, Freemont, CA (US)

(21) Appl. No.: 17/611,298

(22) PCT Filed: Apr. 9, 2020
(86) PCT No.: PCT/US2020/027548

§ 371 Date: Nov. 15, 2021

Related U.S. Application Data
(60) Provisional application No. 62/920,859, filed on May 20, 2019.
(60) Provisional application No. 62/974,317, filed on Nov. 26, 2019.
(60) Provisional application No. 62/995,872, filed on Feb. 18, 2020.

(51) Int. Cl. A61F 2/16; A61F 9/008; G02C 7/04

(52) U.S. Cl. CPC A61F 2/1618; A61F 2/1637; A61F 9/0081; A61F 9/00831; G02C 7/044; G02C 2202/22; A61F 2/164; A61F 2009/00872; A61F 2009/0088; A61F 2009/00895; A61F 2/1624

(57) ABSTRACT

Methods and devices are provided for wavefront treatments of an eye's astigmatism, coma, and presbyopia. Wavefront-engineered monofocal lenses, inducing spherical aberration into the eye's central pupil, provide vision correction beyond 20/20 acuity and improve quality of vision by eliminating image distortion caused by uncorrected astigmatism and coma in the eye. New presbyopia-correcting lenses, including Extended Depth of Focus (EDOF) bifocal, EDOF trifocal, and quasi-accommodating lenses, are disclosed for presbyopia corrections between +0.75 D to +3.25 D, and they are achieved by inducing a positive spherical aberration and a positive focus offset less than 3 Diopters in a central section plus a negative spherical aberration in an annular section within a central part of a monofocal lens. These wavefront lenses can be adapted for contact lenses, implantable contact lenses, Intraocular Lenses (IOLs), phakic IOLs, accommodating IOLs, corneal inlays, as well as eyepieces for Virtual Reality (VR) displays, game goggles, microscopes, telescopes.

### RELATED APPLICATION DATA

The instant application is a national stage bypass CIP application under 35 USC 111(a) of PCT/US2020/027548, filed on Apr. 9, 2020 which claims priority to U.S. provisional applications: 1)# 62/920,859 filed on May 20, 2019 by Junzhong Liang and Ling Yu, titled “Wavefront monofocal lenses, wavefront bifocals, wavefront trifocals, and methods and devices of using spherical aberration to mitigate eye's astigmatism and focus errors,” 2) #62/974,317 filed on Nov. 26, 2019 by Junzhong Liang and Ling Yu, titled “ Methods and devices for wavefront correction of Astigmatism, coma, presbyopia in human eyes,” and 3) #62/995/872 filed on Feb. 18,2020 by Junzhong Liang and Ling Yu, titled “Wavefront monofocal, EDOF bifocal, EDOF trifocal, continuously-in-focus lenses and wavefront correction for astigmatism, coma, presbyopia in human eyes.” The disclosures of these related applications are incorporated herein by reference.

### FIELD OF THE INVENTION

This application relates to refractive correction of human eyes including myopia, hyperopia, astigmatism, coma, and presbyopia in the form of apparatus, methods, and applications.

### BACKGROUND

Conventional refractive corrections for human eyes up until now are designed for the correction of specific refractive errors in eyes: focus errors (myopia and hyperopia), astigmatism (cylinder error), and spherical aberration in some cases. These refractive corrections are compromised for a number of reasons: 1) limitations in selecting a correction device for an astigmatic correction, 2) limitations and errors in measuring an eye's refractive defects using manifset refraction, 3) manufacturing errors in ophthalmic lenses, 4) coma or other high-order aberrations in some eyes.

Presbyopia is another factor that degrades human vision. Most people begin to notice the effects of presbyopia some time after age 40, when they start having trouble clearly seeing small print. Devices for presbyopia correction include reading glasses, bifocal/trifocal/progressive spectacles, multifocal contact lenses, and diffractuve bifocal/trifocal intraocular lenses (IOLs).

Bifocals, invented by Benjamin Franklin in 1824, are eyeglasses with two distinct optical powers. In addition to a baseline power for far vision defects, bifocals also have an add-on power on top of the the baseline power for presbyopia correction. The two distinct optical powers in bifocal spectacles are placed at split physical locations, e.g., at the top for far distances and at the bottom for near distances. When people roll their eyes upward and downward, vision correction for far distances and near distances do not use the same optics in the lens. This split-optics design cannot be employed in contact lenses, IOLs, implantable contact lenses (ICLs), corneal inlays, and surgical procedures because the eye must use the same optics to see objects at far distances and near distances when the freedom of rolling the eye up and down for the two distinct optical powers is lost.

Diffractive optics uses grooved Kinoform steps on top of a monofocal lens to generate 1) a first focus from the non-deviated “0” order diffraction for a far distance and 2) another focus from the deviated “1” order diffraction, creating simultaneous multiple foci from the same incoming light. Diffractive optics has been reported in bifocal (see U.S. Pat. No. 5,116,111) and trifocal IOLs (see U.S. Pat. No. 8,636,796, No. 9,320,594).

Advantages of diffractive bifocal and trifocal IOLs include: 1) solving the problem of split-optics for making bifocal or trifocal lenses, 2) allowing post-op cataract patients to see far distances and near distances without eyeglasses. However, diffractive lenses (bifocal/trifocal IOLs) cannot be tolerated by most post-op cataract patients because they severely degrade quality of vision. Firstly, diffractive bifocal/trifocal IOLs cause nighttime symptoms such as halo and starburst due to multiple images of bright objects at far distances. Secondly, spider-web night symptoms are often seen, caused by diffraction rings projected onto the retina.

Diffraction optics cannot be applied to contact lenses because the diffractive surface, which is not continuous and contains sharp edges (see FIG. 1), would cause tissue damage to the corneal surface or disrupt normal tear flow on the cornea. Since both the split-optics design in bifocal spectacles and the diffraction-optics in IOLs are not suitable for contact lenses, there currently is no reliable bifocal contact lens in the prior art even though many multifocal contact lenses are commercially available. Multifocal contact lenses that rely upon pupil-splitting for presbyopia corrections are reported (see U.S. Pat. No. 6,808,262, No. 4,704,016, No. 4,898,461, No. 4,704,016, No. 6,808,262). Retinal images for both far distances and near distances are uncertain if physical optics is considered, e.g., diffraction and interference of light beams across the pupil of an eye.

The ultimate solution for fixing presbyopia for human vision is either to restore accommodation of an aged crystalline lens in the eye or to replace the optics of an eye with an accommodating IOL. After tremendous effort in developing accommodating IOLs over the last 20 years, progress has been made recently in achieving accommodation by fluid IOLs (see FIG. 2). However, analysis of the data of accommodating IOLs indicates at least three issues that are clinically significant. First, there is a large fluctuation in the focus power, which is as large as +/−0.5 D, at both targeted accommodation states for far distances around 0 D and for near distances around 3 D for eyes E13-401 (top right in FIGS. 2) and E15-301 (bottom right in FIG. 2). Second, at the far accommodation state, the accommodating IOLs can have a mean accommodation error of −1.0 D for eye E13-401 (top right in FIG. 2) at the time scale of 0 to 5 seconds and for eye E02-411 (bottom left in FIG. 2) at time scales around 15 seconds and 25 seconds. This large focus error can result in difficulty seeing clearly at the far distances from time to time. Third, the accommodation range in the eyes in FIG. 2 varies from eye to eye and from moment to moment for some eyes.

U.S. Pat. No. 8,529,559 B2 and US patent application # 2011/0029073 A1 disclosed methods and devices of inducing spherical aberration into eye's central pupil for presbyopia treatments. While providing the benefit of extending depth of focus for ophthalmic lenses, inducing spherical aberration by corrective lenses is believed to reduce retina contrast significantly. Inducing spherical aberration of opposite sign into the eye's central pupil is also proposed to extend depth of focus up to 3.5 D. Unfortunately, the original designs suffer from significantly reduced contrast at far distances.

Consequently, although many configurations and methods for vision correction are known in the art, these conventional methods and systems suffer from one or more disadvantages discussed herein above.

### SUMMARY

In a non-limiting embodiment, a wavefront-engineered monofocal lens for an eye, configured as an implantable lens or a wearable lens, includes a) a baseline Diopter power extending across an optical section with a diameter between 5 mm and 8 mm for a spherocylindrical correction; b) at least an aspherical section having at least one aspheric surface in the center of the monofocal lens with a diameter D₀ between 2.5 mm and 4.5 mm, wherein the aspherical section induces spherical aberration into the eye's central pupil, and the induced spherical aberration or wavefront error in the lens center provides treatments for residual refractive errors in the eye left uncorrected by the spherocylindrical correction, wherein the residual and uncorrected refractive errors include astigmatism, focus errors, coma and higher order aberrations that are significant in the central pupil of the eye. In a non-limiting embodiment, a bifocal lens for an eye configured as an implantable lens or a wearable lens, includes a baseline Diopter power extending across an optical section with a diameter between 5 mm and 8 mm for a spherocylindrical correction; a positive focus offset ϕ₁ at a center section having a diameter less than 2.5 mm and larger than 1.8 mm, wherein the positive focus offset is less than +2.0 D and more than +0.25 D; two central aspherical sections at least in the center of the lens having an outer diameter less than 4.5 mm and larger than 2.5 mm, wherein the central aspherical sections comprises at least one aspheric surface for inducing a positive spherical aberration in a first zone and a negative spherical aberration in a second zone, wherein the first zone and the second zone are concentric. In a non-limiting embodiment, a trifocal lens for an eye configured as an implantable lens or a wearable lens, includes a baseline Diopter power extending across an optical section with a diameter between 5 mm and 8 mm for a spherocylindrical correction; a positive focus offset ϕ₁ at a center section having a diameter D₀ less than 2.1 mm and larger than 1.65 mm, wherein the positive focus offset is less than +3.0 D and larger than +1.0 D; two central aspherical sections at least in the center of the lens having an outer diameter less than 4 mm and larger than 2.5 mm, wherein the central aspherical sections comprises at least one aspheric surface for inducing a positive spherical aberration in a first zone and a negative spherical aberration in a second zone, wherein the first zone and the second zone are concentric; wherein the wavefront errors from the induced focus offset ϕ₁ and induced spherical aberrations in the central aspherical sections create a trifocal lens: a first “far” focus, a second focus with an “intermediate” add-on power, and a third focus with a “near” add-on power, wherein the positive focus offset ϕ₁ at the center section must be less than the total focus range of the trifocal lens.

In a non-limiting embodiment, a Continuously-In-Focus (CIF) lens for an eye has an optical section less than 8 mm in diameter including a multifocal structure that provides a continuous focus for vision correction in a focus range larger than 1.0 D, wherein the multifocal structure has multiple foci immediately adjacent each other to provide a substantially continuous focus; wherein the multiple foci are achieved either by using an aspherical surface to induce spherical aberrations into the central part of lens with a diameter less than 4 mm or using diffractive optics to create simultaneous multiple foci.

In a non-limiting embodiment , a wavefront Implantable Contact Lens (ICL) for an eye comprises: a haptics section for fixing the ICL to an iris in an anterior chamber of an eye or holding the ICL in place inside a posterior chamber of an eye; an optical lens section including i) a baseline Diopter power extending across an optical section with a diameter between 5 mm and 8 mm for a spherocylindrical correction, ii) a central section with a diameter between 1.65 mm and 2.5 mm that induces a positive spherical aberration plus a positive focus offset ϕ1 less than +3.0 D and greater than +0.5 D, iii) an annular section with an outer diameter less than 4.5 mm that induces a negative spherical aberration; wherein the wavefront errors from the induced spherical aberrations and the focus offset in the central and annular sections make the optical lens one of i) a quasi-accommodation and continuous-in focus lens, ii) a wavefront bifocal lens, iii) a wavefront trifocal lens.

In a non-limiting embodiment, a method of refractive correction for an eye comprises the steps of: determining refractive errors of an eye for a far vision correction, wherein the refractive errors include at least a sphere power SPH; performing a refractive surgery of an Extended Depth of Focus between a first focus power ϕ₁ and a second focus power ϕ₂ and the targeted spherical power SPH is set between the first focus power ϕ₁ and the second focus power ϕ₂ so that the post-op eye can retain excellent vision at far distances even if the post-op eye develops myopia progression in the future.

### BRIEF DESCRIPTION OF THE DRAWINGS

FIG. 1 illustrates a cross-section view of a difractive bifocal IOL (top) and a diffractive trifocal IOL (bottom) in the prior art.

FIG. 2 shows objective measurements of accommodation of acccommodating IOLs in eyes in the prior art.

FIG. 3 shows parameters of toric contact lenses in the prior art.

FIG. 4 shows specification parameters of toric IOLs in the prior art.

FIG. 5A shows point spread functions of a hypothetical eye for a pupil diameter of 3.5 mm with astigmatism (CYL) betweem 0 D and ⅝ D and a focus error (SPH) between −0.5 D and +0.5 D left uncorrected by a conventional monofocal contact lens or a conventional monofocal IOL.

FIG. 5B shows the calculated retinal images of the hypothetical eye for a pupil diameter of 3.5 mm with astigamtism betweem 0 D and ⅝ D and a focus error (SPH) between −0.5 D and +0.5 D left uncorrected by a conventional monofocal contact lens or a conventional monofocal IOL. Tumbling E is calibrated for visual acuity of 20/16 (smallest letters), 20/20, 20/25, 20/30, and 20/40 (largest letters).

FIG. 6A shows point spread functions of a hypothetical eye for a pupil diameter of 3.5 mm with astigmatism (CYL) of ⅝ D and a focus error (SPH) between −0.5 D and +0.5 D left uncorrected by a monofocal contact lens or a monofocal IOL. In addition, six scenarios of spherical aberration in a corrected eye are provided, including 1) S₁=0, meaning a perfect correction of spherical aberration existing in a natural eye, 2) S₁=−0.26, meaning no change of spherical aberration in a natural eye, 3) S₁=−0.52, −0.78, −1.04, −1.3, meaning that more spherical aberration is induced into the eye.

FIG. 6B shows the calculated retinal images from the point spread functions for the cases in FIG. 6A.

FIG. 6C shows point spread functions of a hypothetical eye for a pupil diameter of 3.5 mm with astigmatism (CYL) of ⅝ D and a focus error (SPH) between −0.5 D and +0.5 D left uncorrected by a monofocal contact lens or a monofocal IOL. In addition, six scenarios of eye's spherical aberration are provided, which include 1) S₁=0, 2) S₁=0.26, and 3) S₁=0.52, 0.78,1.04, 1.3, meaning that more spherical aberration is induced into the eye.

FIG. 6D shows the calculated retinal images from the point spread functions for the cases in FIG. 6C.

FIG. 6E shows point spread functions of a hypothetical eye for a pupil diameter of 3.5 mm with astigmatism (CYL) of ⅜ D and a focus error (SPH) between −0.5 D and +0.5 D left uncorrected by a monofocal contact lens or a monofocal IOL. In addition, six scenarios of eye's spherical aberration are considered, which include 1) S₁=0, 2) S₁=−0.26, and 3) S₁=−0.52, −0.78,−1.04,−1.3, meaning that more spherical aberration is induced into the eye.

FIG. 6F shows the calculated retinal images from the point spread functions for the cases in FIG. 6E.

FIG. 6G shows point spread functions of a hypothetical eye for a pupil diameter of 3.5 mm with no astigmatism (CYL=0D) and a focus error (SPH) between −0.5 D and +0.5 D left uncorrected by a monofocal contact lens or a monofocal IOL. In addition, six scenarios of eye's spherical aberration are considered, which include 1) S₁=0, 2) S₁=−0.26, and 3) S₁=−0.52, −0.78,−1.04,−1.3, meaning that more spherical aberration is induced into the eye.

FIG. 6H shows the calculated retinal images from the point spread functions for the cases in FIG. 6G.

FIG. 6I shows calculated retinal images of an acuity chart for a hypothetical eye with only coma left uncorrected by a conventional monofocal lens (left column) and by a wavefront-enginered monofocal lens in one examplary design (right colum) for a 3.5 mm pupil. Coma in the eye is measured by a Zenike polynomail with a coefficient of 1.0 microns for a 6 mm pupil. Coma in three different orientations are considered.

FIG. 6J shows calculated retinal images of an acuity chart for a hypothetical eye with only coma left uncorrected by a conventional monofocal lens (left column) and by a wavefront-engineered monofocal lens in one examplary design (right colum) for a 3.5 mm pupil. Coma in the eye is measured by a Zenike polynomail with a coefficient of 1.5 microns for a 6 mm pupil. Coma in three different orientations are considered.

FIG. 7 shows a schematic diagram of a wavefront-engineered monofocal lens in one aspect of the present invention.

FIG. 8A shows point spread functions of a hypothetical eye for a pupil diameter of 3.5 mm for an conventional monofocal lens (left column) in comparison to an exemplary wavefront-engineered monofocal lens (right column) in the present invention. Eye's astigmatish is assumed to be zero or perfectly corrected (CYL=0). A focus error (SPH) between −0.5 D and +0.5 D is left uncorrected by the monofocal lenses.

FIG. 8B shows calculated retinal images from the point spread functions in FIG. 8A with the conventional monofocal lens (left column) in comparison to the wavefront-engineered monofocal lens (left column) in the exemplary design.

FIG. 8C shows calculated Modulation Transfer Functions (MTF) from the point spread functions in FIG. 8A for the conventional monofocal lens (Top) in comparison to the wavefront-engineered monofocal lens in the exemplary design (bottom).

FIG. 9A shows point spread functions of a hypothetical eye for a pupil diameter of 3.5 mm with an exemplary wavefront-engineered monofocal lens in Table 2A. Astigmatism (CYL) betweem 0 D and ⅝ D and a focus error (SPH) between −0.5 D and +0.5 D are left uncorrected by the wavefront-engineered monofocal lens.

FIG. 9B shows the calculated retinal images for the same hypothetical eye for pupil diameter of 3.5 mm (indoor and acuity test) with an exemplary wavefront-engineered monofocal lens in Table 2A.

FIG. 9C shows the calculated retinal images for the same hypothetical eye for a pupil diameter of 2.5 mm (outdoor and day vision) with the wavefront-engineered monofocal lens in Table 2A.

FIG. 9D shows the calculated retinal images of a hypothetical eye for a pupil diameter of 5 mm (night vision) with the wavefront-engineered monofocal lens in Table 2A.

FIG. 9E shows the calculated retinal images of a hypothetical eye for a pupil diameter of 5 mm (night vision) with a conventional monofocal lens.

FIG. 9F shows point spread functions of a hypothetical eye for a pupil diameter of 3.5 mm with a wavefront-engineered monofocal lens in another exemplary design (Table 2B). Astigmatism (CYL) betweem 0 D and ⅝ D and a focus error (SPH) between −0.5 D and +0.5 D are left uncorrected by the wavefront-engineered monofocal lens.

FIG. 9G shows the calculated retinal images from the point spread functions for the cases in FIG. 9F.

FIG. 10A shows calculated point spread functions of a hypothetical eye with a “PureVision-low” multifocal lens from Bausch & Lomb for pupil diameters of 3.0 mm, 3.5 mm, 4.5 mm and 5 mm. For simplicity, we consider CYL=0 D only.

FIG. 10B shows the calculated retinal images of a hypothetical eye with a “PureVisionlow” multifocal lens from Bausch & Lomb.

FIG. 10C shows point spread functions of a hypothetical eye with an “Air Optix-med” multifocal lense from Alcon for pupil diameters of 3.0 mm, 3.5 mm, 4.5 mm and 5 mm. For simplicity, we consider CYL=0 D only.

FIG. 10D shows the calculated retinal images of a hypothetical eye with an “Air Optix-med” multifocal lense from Alcon.

FIG. 11 shows a schematic diagram of a wavefront bifocal, trifocal, continously-in-focus lens in one aspect of the present invention.

FIG. 12A shows point spread functions of a hypothetical eye with an examplary design of wavefront bifocal lense (WF Bifocal 1 D) for pupil diameters of 3.0 mm, 3.5 mm, 4.5 mm and 5 mm. For simplicity, we consider the case of CYL=0 D.

FIG. 12B shows the calculated retinal images from the point spread functions in FIG. 10A with our design of wavefront bifocal lense (WF Bifocal 1 D).

FIG. 12C shows plots of calculated retinal contrast “through focus” of WF Bifocal 1 D for a 3.5 mm pupil, and for 20/20 lines and 20/40 lines with pupil size between 3 mm to 5 mm.

FIG. 12D shows calculated retinal contrast for 20/25, 20/30, 20/40, 20/60 for normal eyes in a photopic condition (A) and in Mesopic condition (B) from studing more than 250 eyes of US navy pilots with 5% low contrast acuity for photopic vision and with 25% low contrast acuity for mesopic vision.

FIG. 12E shows plots of calculated Modulation Transfer Function (MTF) of WF Bifocal 1 D for far distances at infinity (−0.25 D), at 4 meters (0 D), and a focus error at +0.25 D for pupil sizes of 3 mm, 3.5 mm, and 5 mm.

FIG. 13A shows point spread functions of a hypothetical eye with our design of wavefront EDOF Bifocal 3 D for pupil diameters of 3.0 mm, 3.5 mm, 4.5 mm and 5 mm. For simplicity, we consider the case of CYL=0 D only.

FIG. 13B shows the calculated retinal images from the point spread functions in FIG. 13A with our wavefront EDOF Bifocal 3 D lens.

FIG. 13C shows plots of calculated retinal contrast “through focus” of EDOF Bifocal 3 D for a 3 mm pupil, and for 20/20 lines and 20/40 lines with pupil size between 3 mm to 5 mm.

FIG. 13D shows plots of calculated Modulation Transfer Function (MTF) of EDOF Bifocal 3 D for far distances at infinity (−0.25 D), at 4 meters (0 D), and a focus error at +0.25 D for pupil sizes of 3 mm, 3.5 mm, and 5 mm.

FIG. 13E shows calculated retinal contrast for far distances in (A) as well as through-focus for 20/20 acuity in (B) of our EDOF Bifocal 3 D in comparion to the wavefront design in the prior art.

FIG. 14A shows point spread functions of a hypothetical eye with one design of wavefront “EDOF Trifocal 2.75 D” for pupil diameters of 3.0 mm, 3.5 mm, 4.5 mm and 5 mm. For simplicity, we consider the case of CYL=0 D only.

FIG. 14B shows the calculated retinal images from the point spread functions in FIG. 14A with a wavefront “EDOF Trifocal2.75 D” lens.

FIG. 14C shows plots of calculated retinal contrast “through focus” of EDOF Trifocal2.75 D for a 3 mm pupil, and for 20/20 lines and 20/40 lines with pupil size between 3 mm to 5 mm.

FIG. 14D shows plots of calculated Modulation Transfer Function (MTF) of EDOF Trifocal2.75 D for far distances at infinity (−0.25 D), at 4 meters (0 D), and a focus error at +0.25 D for pupil sizes of 3 mm, 3.5 mm, and 5 mm.

FIG. 15A shows point spread functions of a hypothetical eye with one design of wavefront Quasi Accommodating and Continously-in-Focus “QACIF2D” for pupil diameters of 3.0 mm, 3.5 mm, 4.5 mm and 5 mm. For simplicity, we consider the case of CYL=0 D only.

FIG. 15B shows the calculated retinal images from the point spread functions in FIG. 15A with the wavefront QACIF2D lens.

FIG. 15C shows plots of calculated retinal contrast “through focus” of QACIF2D for a 3.5 mm pupil, and for 20/20 lines and 20/40 lines with pupil size between 3 mm to 5 mm.

FIG. 15D shows plots of calculated Modulation Transfer Function (MTF) of QACIF2D for far distances at infinity (−0.25 D), at 4 meters (0 D), and a focus error at +0.25 D for pupil sizes of 3 mm, 3.5 mm, and 5 mm.

FIG. 15E shows plots of calculated retinal contrast “through focus” of QACIF2A for a 3.5 mm pupil, and for 20/20 lines and 20/40 lines with pupil size between 3 mm to 5 mm.

FIG. 15F shows the calculated retinal images with the wavefront QACIF2D lens if CYL=½ D.

FIG. 15G shows the calculated retinal images with the wavefront QACIF2D lens if CYL=¾ D.

FIG. 16 provides a comparison of wavefront mono/multifocal lenses in the present invention with conventional refractive monofocal lenses, difractive monofocal/multifocal lenses for night vision as well as quality of vision impacted by imperfect corrections of astigmatism and focus error by these ophthalmic lenses.

FIG. 17A shows calculated retinal imaged for a pupil size of 5 mm at nighttime for a conventional refractive monofocal lenses in comparison with several emaplary designs of wavefront multifocal lenses in the present inventions at far infinity (−0.25 D), at 4 meters (0 D), and a focus error at +0.25 D.

FIG. 17B shows image principle of a diffractive bifocal lens in (A) as well as components of calculted retinal images at far distances for diffractive bifocal lens with an add-on power of +1.75 D in (B) and 3.5 D in (C), respectively.

FIG. 17C shows calculate retinal images of a monofocal lens through focus between −0.75 D and +0.75 D with uncorrected astigmatism of ⅜ D.

FIG. 18 illustrates a liquid ophthalmic lens in one aspect of the present invention.

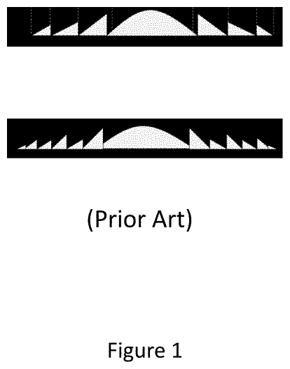

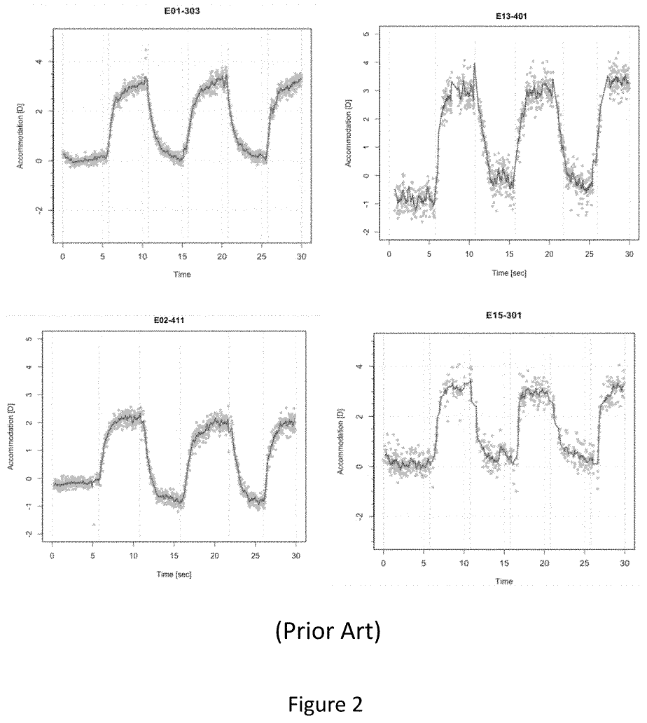

### DETAILED DESCRIPTION

### 1. Wavefront-Engineered Monofocal/Toric Lenses

Focus errors (SPH) and astigmatism (CYL) are refractive errors in human eyes that cause image blur and degrade visual acuity and quality of vision.

Monofocal lenses, also called single vision lenses, are the most common forms of eyeglasses, contact lenses, implantable contact lenses, and IOLs. Types of monofocal lenses include spherical monofocal lenses, aspherical monofocal lenses, and toric monofocal lenses.

Spherical monofocal lenses use spherical surfaces for both the front and the back surfaces and are used for correction of focus errors in the eye such as myopia and hyperopia.

Toric monofocal lenses use at least one toric surface; they not only provide vision correction for focus errors but also astigmatism in an eye.

### 1A. Astigmatism Left Uncorrected by Monofocal/Toric Ophthalmic Lenses

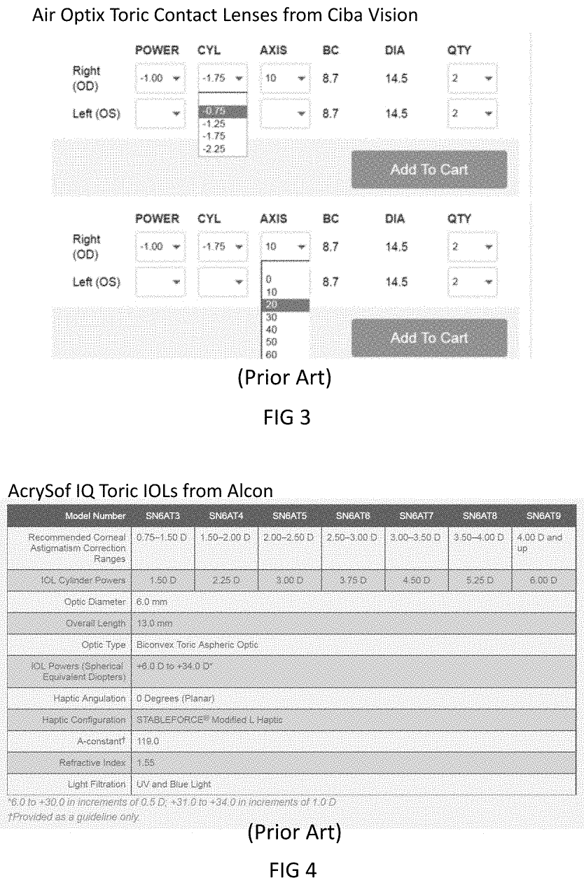

Correction of astigmatism by toric contact lenses usually starts from 0.75 D, with an incremental step of 0.5 D. This is shown in FIG. 3, which is an online order form for Air Optix toric contact lenses from Ciba Vision and Alcon Laboratories, Inc. Astigmatic corrections by IOLs also start from about 0.75 D. FIG. 4 shows specifications of AcrySof® IQ Toric IOLs as well as the guidelines for using these toric IOLs from Alcon Laboratories, Inc. The recommendation shows that astigmatism of 0.75 D to 1.0 D can be left uncorrected by toric monofocal IOLs.

Error sources for a astigmatic correction in contact lenses, Implantable Contact Lenses (ICLs), IOLs include: 1) astigmatism that is not corrected in the prescription if the eye's astigmatism is less than 0.75 D, determined in eye refraction, 2) a limited selection of toric powers in toric lenses with incremental steps of 0.5 D, 3) selection of a toric AXIS is limited to 10 degree increments, 4) rotation of toric contact lenses on a cornea or rotation of toric ICLs and IOLs in post-op settlement.

Therefore, astigmatism in human eyes has not been well corrected by either existing monofocal or toric lenses that include contact lenses, IOLs, ICLs. Uncorrected astigmatism left in eyes can be as much as ⅝ D.

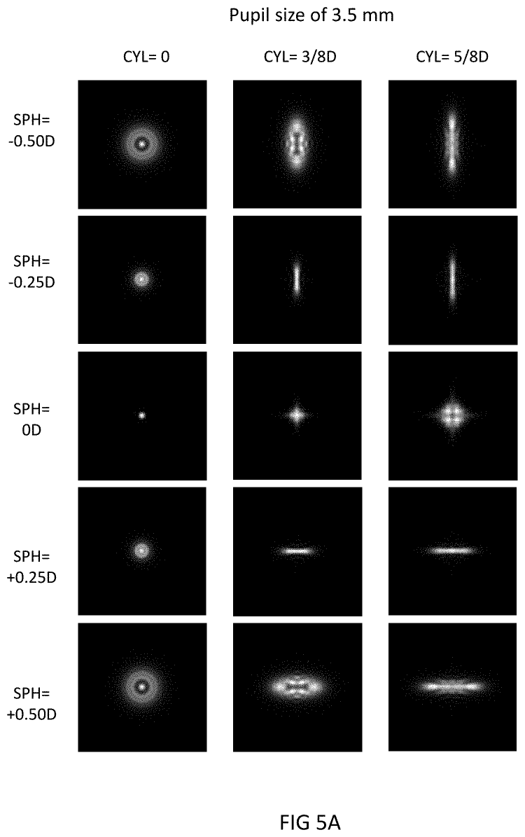

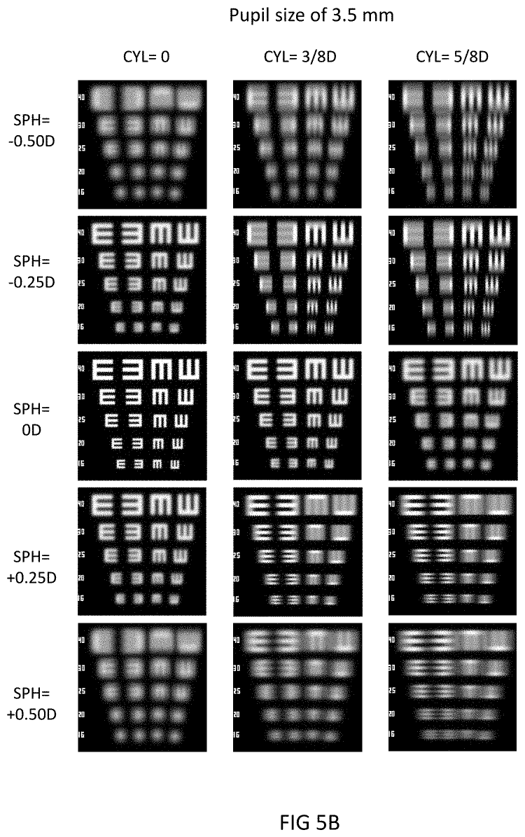

In order to study the impact of eye's uncorrected astigmatism left by conventional monofocal lenses, we provide a simulation of the eye's point-spread functions in FIG. 5A as well as the simulated retinal images of acuity charts in FIG. 5B.

In the simulation, we considered a perfect correction of astigmatism (CYL=0) and two cases with uncorrected astigmatism of ⅜ D and ⅝ D. We also considered an uncorrected focus error (SPH) of −0.5 D, −0.25 D, 0D, +0.25 D, and +0.5 D because uncorrected focus errors are also common for IOLs, ICLs, and contact lenses. Error sources include 1) a myopic power of −0.25 D between far vision at infinity and far vision at 4 meters for refractive testing, 2) a limited selection in SPH power for IOLs and ICLs, 3) errors in SPH power of the ordered lenses, 4) errors in eye refraction.

FIG. 5A shows retinal images of a point source, or point-spread functions, of a hypothetical eye for a pupil size of 3.5 mm in diameter. Significant image blurs are seen in FIG. 5A except for the case of a perfect correction (SPH=0 and CYL=0). From the calculated point-spread functions in FIG. 5A, we calculated the corresponding retinal images of an acuity chart of the eye in FIG. 5B, by convolving the calculated point-spread functions in FIG. 5A with a tumbling E acuity chart. The acuity chart consists of letter Es in different sizes, calibrated for visual acuity of 20/16 (the smallest letters and at the bottom row in each image in FIG. 5B), 20/20, 20/25, 20/30, and 20/40 (the largest letters and on the top row in in each image).

It must be pointed out that the total dimension size for the point-spread functions in FIG. 5A is ⅛ of that for the retinal images in FIG. 5B in order to show the fine details of the point-spread functions.

All the simulated point-spread functions in this disclosure have the same dimensional scale while all the simulated retinal images in this disclosure have the same dimensional scales as well, and the dimensional scales of point-spread functions are ⅛ as large as that for the retinal images. We use the same acuity chart in simulations for all the cases throughout this application, consisting of letter Es in different sizes, calibrated for visual acuity of 20/16 (the smallest letters and at the bottom row in each image in FIG. 5B), 20/20, 20/25, 20/30, and 20/40 (the largest letters and on the top row in in each image in FIG. 5B).

From the simulation results in FIG. 5A and FIG. 5B, it can be seen that conventional monofocal contact lenses, ICLs, IOLs are far from adequate. Quality of vision is only good if both SPH and CYL are nearly perfectly corrected. Several issues are noticed.

Firstly, when astigmatism is not properly corrected, image blur due to astigmatism such as CYL=⅝ D (3^{rd} column in FIG. 5A and 5B) will make it impossible to recognize a complete set of acuity letters for 20/20 (the 2^{nd} smallest letters in the chart) in any one of the five focus SPH settings. Because of this, people will most likely have compromised vision, and their best corrected acuity is in the range of 20/40 or 20/30 (the largest or the second letters in the chart) instead of normal acuity of 20/20.

Secondly, even in the case when astigmatism is perfectly corrected (CYL=0, 1^{st} column in FIG. 5A and 5B), vision is blurred so that a 20/16 letter (smallest letter in the chart) is no longer resolvable if there is a focus error of +/−0.25 D. Vision is totally blurred for all letters from 20/40 to 20/16 if the focus error is +/−0.5 D. This is significant because vision is tested at 4 meters indoors while a myopic SPH error of −0.25 D will occur for outdoors at infinity.

Thirdly, image distortion (structure change between objects and their images) is clearly observed if uncorrected astigmatism is coupled with uncorrected focus error +/−0.25 D or if the uncorrected focus error alone reaches a level of 0.5 D.

Finally, toric lenses will have the same issues because their correction for astigmatism is limited as shown in FIG. 3 and FIG. 4.

### 1B. Spherical Aberration in Normal Human Eyes

In spherical aberration, parallel light rays that pass through the central region of a positive lens focus farther away than light rays that pass through the edges of the lens. The optics of a human eye is a positive lens, and spherical aberration is significant at the pupil periphery. Based on a study of 214 eyes, the Zernike spherical aberration (2.236*(6 r⁴−6 r²+1)) is found to be +0.138±0.103 microns for a 5.7 mm pupil, where r is a normalized pupil radius (r=ρ/2.85) and ρ is the pupil radius of the eye (J. Porter et. al., Monochromatic aberrations of the human eye in a large population, Journal of the Optical Society of America A, Vol. 18, issue 8, pp. 1793-1803 (2001)).

From Porter's mean Zernike spherical aberration W₁₂(ρ)=0.138*2.236*6*(r⁴−r²+1), we would obtain its corresponding Seidel spherical aberration W(ρ)=1.85*r⁴=1.85*(ρ/2.85)⁴, or

$$W ( ρ ) = 0.0280 {ρ}^{4}$$

Diopter power profile ϕ(ρ) can be derived from Seidel spherical aberration W(ρ) as

$$ϕ ( ρ ) = - ( dW ( ρ ) \text{/} d ρ ) \text{/} ρ = - 0.11 * {ρ}^{2}$$

where ρ is a polar radius in millimeters. We believe the coefficient for Zernike spherical aberration from Porter et. al. is its correction instead of the Zernike spherical aberration itself because 1) it is well-known that eye's refraction power is higher at pupil periphery than that at the pupil center for human eyes, 2) the Diopter power (−0.11±0.08 D/mm²) is close to the Diopter profiles of 0.10±0.06 D/mm² provided by S. Plainis, D A Atchison and W N Charman in “Power Profiles of Multifocal Contact Lenses and Their Interpretation,” in Optometry and Vision Sciences, vol. 90, No. 10, pp1066-1077) with an opposite sign.

Therefore, we take a negative Seidel spherical aberration in normal eyes as

$$\begin{matrix}W ( ρ ) = - 1.85 * {( ρ \text{/} 2.85 )}^{4} \\ = - 0.0280 {ρ}^{4} ,\end{matrix}$$

and the corresponding focus profile across pupil radius is

$$ϕ ( ρ ) = 0.11 * {ρ}^{2} .$$

It must be also mentioned that S. Plainis, D A Atchison and W N Charman classified the eye's Seidel spherical aberration as “positive,” and this conflicts with the classical definition in Optics (see Modern Optical Engineering by Warren J. Smith on page 65 in the third edition). Positive spherical aberration is called over-corrected and is generally associated with divergent elements (negative lenses) while negative spherical aberration is called under-corrected and is generally associated with convergent elements (positive lenses).

Human eyes have negative spherical aberration, and the wavefront error due to the eye's negative spherical aberration can also be expressed as

$$W ( ρ ) = {S}_{1} * {( ρ \text{/} {r}_{0} )}^{4}$$

where r₀=0.5*D₀ is a pupil radius, ρ is a polar radius in a pupil plane and has a value between 0 and r₀, and a negative spherical aberration has a negative coefficient of S₁ (S₁<0). Table 1 lists the eye's spherical aberration both in microns (μm) and in wavelengths (λ=0.55 microns) for four different pupil sizes: 5.7 mm, 3.5 mm, 3 mm, and 2 mm. The mean spherical aberration in human eyes is −0.26 microns for a 3.5 mm pupil.

TABLE 1
Spherical Aberration of Human Eyes for Different Pupil Sizes

| | | | | | |
|---|---|---|---|---|---|
| Pupil Diameter (mm) | D | 5.7 | 3.5 | 3 | 2 |
| Pupil Radius (mm) | r₀ | 2.85 | 1.75 | 1.5 | 1 |
| Mean Spherical | S₁ | −1.85 | −0.26 | −0.14 | −0.03 |
| Aberration (μm), −1.85 (ρ/r₀)⁴ | | | | | |
| Mean Spherical | S₁ | −3.366 | −0.479 | −0.258 | −0.051 |
| Aberration (λ = 0.55 μm) | | | | | |

It is clearly seen in Table 1 that the eye's spherical aberration is negligible in the central pupil, only about λ/20 within a pupil of 2 mm in diameter and λ/4 within a pupil of 3 mm in diameter, respectively. Optic elements are often considered diffraction-limited or perfect if the wavefront error is below λ/4. On the other hand, the mean spherical aberration in normal human eyes reaches 3.4λ for a large pupil of 5.7 in diameter in the dark, and is thus significant in degrading vision at night.

Aspherical monofocal lenses, using at least one aspheric surface for the front and back surfaces, can also be found in contact lenses and IOLs. The aspherical surface is used fortwo purposes: 1) providing correction for spherical aberration in human eyes that is significant at the pupil periphery, 2) eliminating spherical aberration in IOLs with a large refractive power. In both these cases, aspherical monofocal lenses differ from spherical monofocal lenses only in lens periphery outside roughly a 3 mm diameter, because spherical aberration for human eyes and the correction lenses are insignificant in the central optical zone.

1C. Mitigation of Astigmatism by Inducing Spherical Aberration into Eye's Central Pupil

In one aspect of the present invention, we describe a fundamental discovery about benefits of inducing more spherical aberration in eye's central pupil for improving quality of ophthalmic lenses.

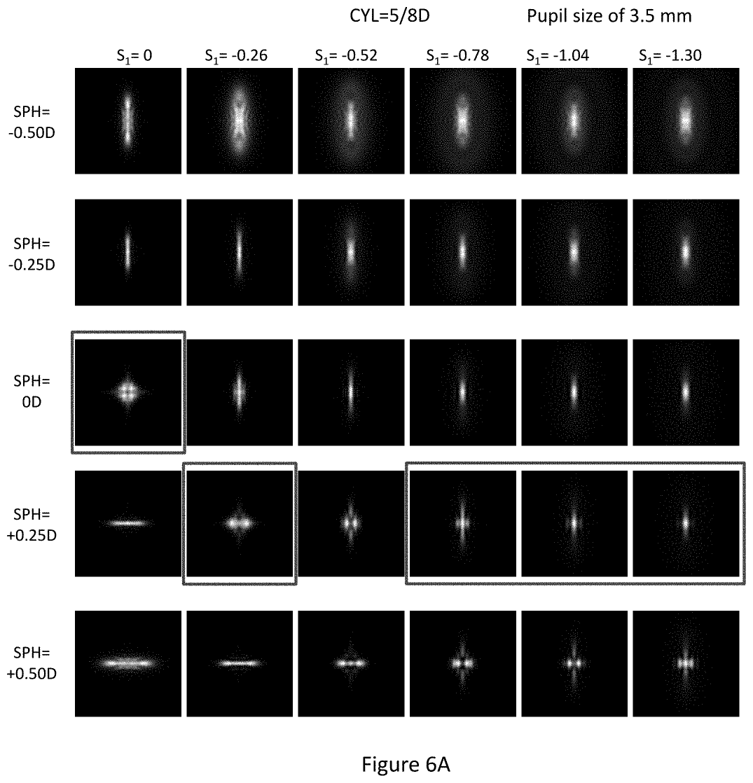

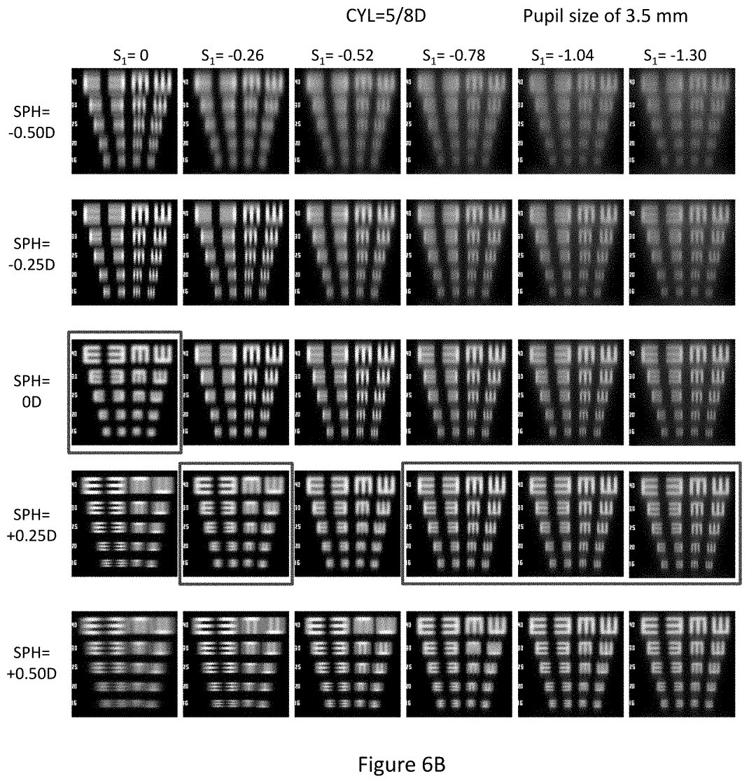

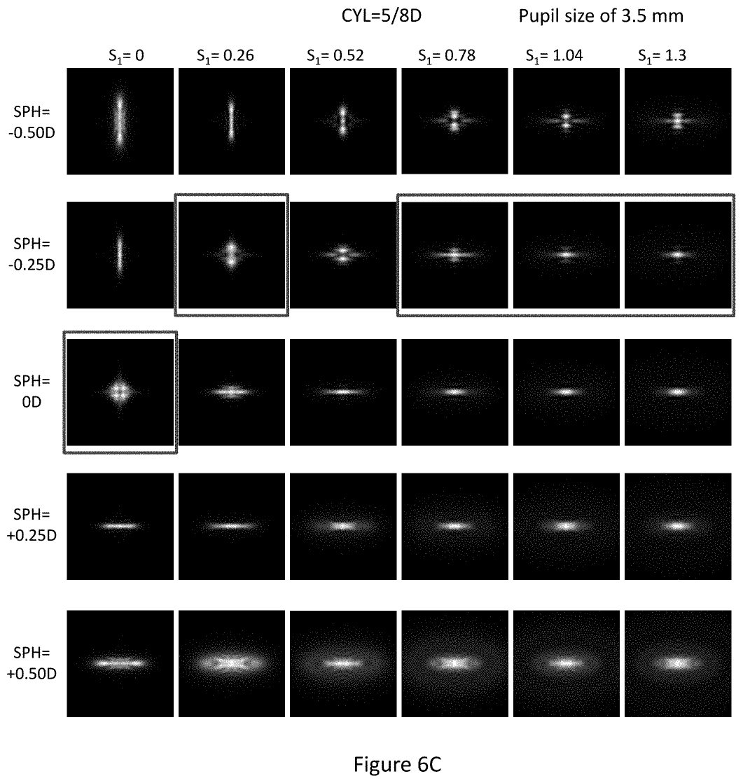

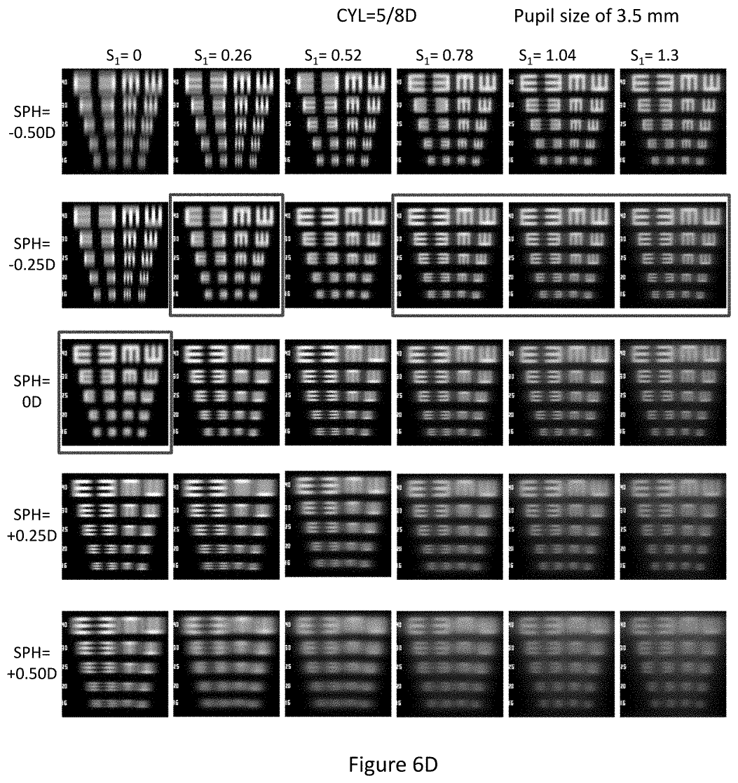

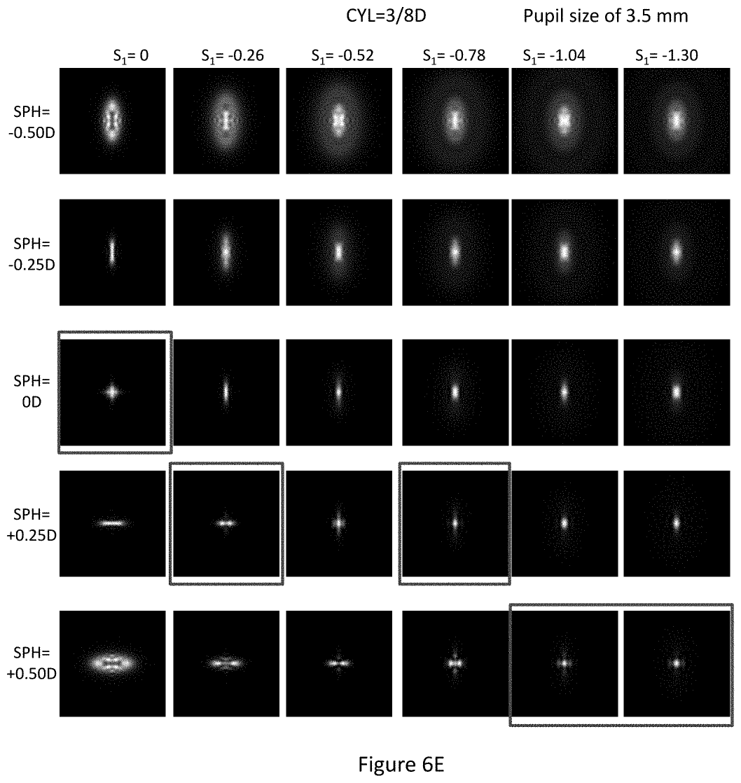

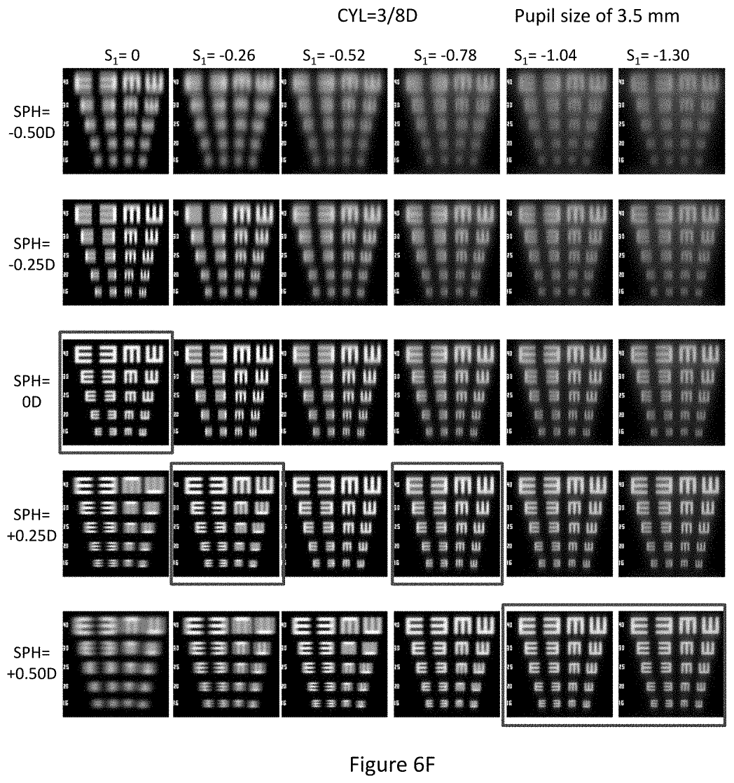

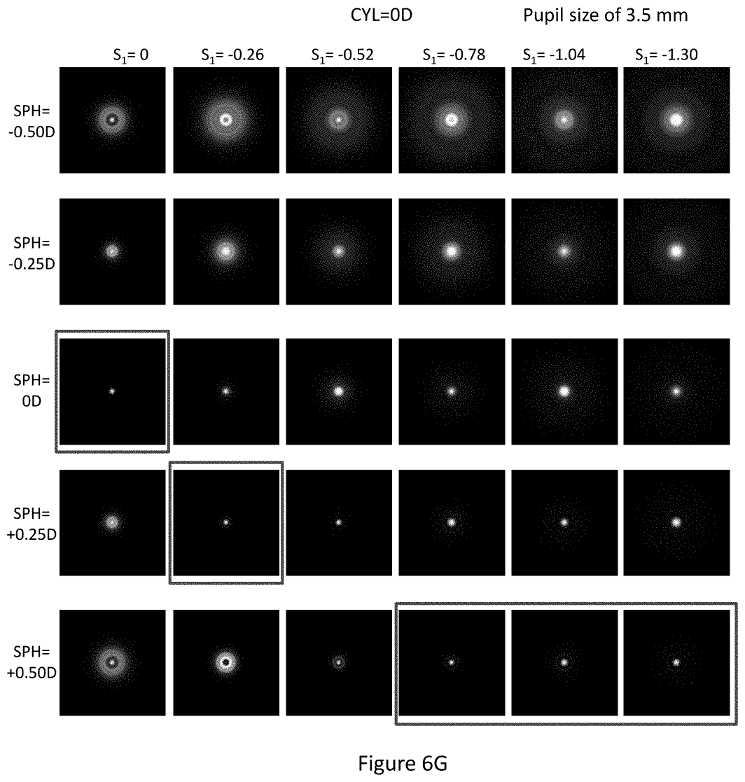

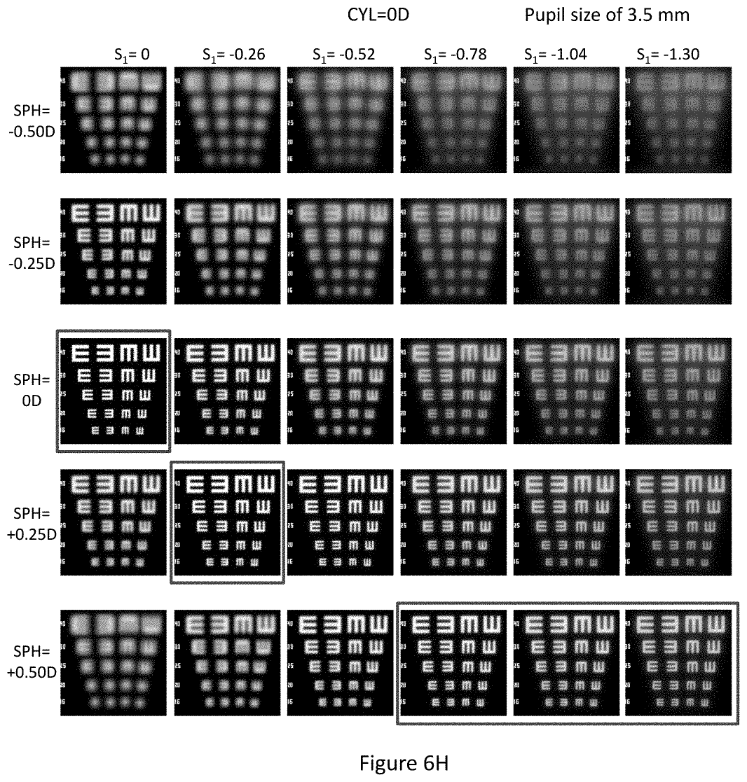

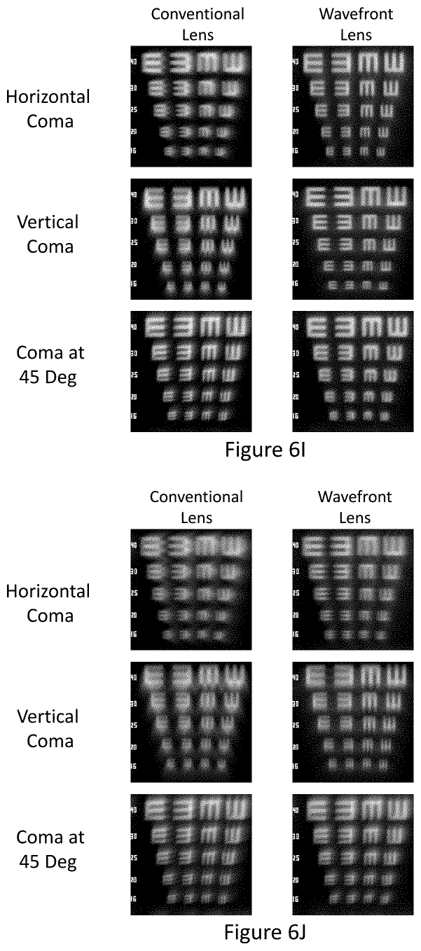

FIG. 6A show point-spread functions for a hypothetical human eye for a pupil size of 3.5 mm in diameter with uncorrected astigmatism of CYL=⅝ D, while six cases of spherical aberration in the eye are considered: 1) S₁=0 (first column from the left) if eye's spherical aberration is completely corrected by a conventional aspherical lens, 2) S₁=−0.26 (second column from the left) if eye's spherical aberration is left unchanged by a spherical lens, 3) S₁=−0.52, −0.78, −1.04, and −1.34 if additional spherical aberration is induced into an eye by a wavefront-engineered lens. A wavefront-engineered monofocal lens in the present invention includes 1) a standard spherocylindrical correction across an optical section having a diameter between 5 mm and 8 mm, 2) induced spherical aberration in the central part of the lens with a diameter between 2.5 mm and 4.5 mm. Vision quality of an eye for a pupil size of 3.5 mm in diameter is simulated because it is the mean pupil size of normal human eyes in clinical test of visual acuity. In the simulation, we also considered different amounts of focus errors (SPH): −0.5 D, −0.25 D, 0D, 0.25 D, 0.5 D.

It is clearly seen that, if the eye has an astigmatism of ⅝ D left uncorrected by a mono-focal contact lens, ICL, or IOL, the eye's point spread function in FIG. 6A is large in size when eye's spherical aberration is completely corrected for S₁=0 or unchanged for S₁=−0.26. The eye's point spread function is more compact and reduced in its size when more spherical aberration is induced into the central pupil in the case from S₁=−0.52 to S₁=−1.3.

From the point spread function in FIG. 6A, we calculated eye's retinal images of an acuity chart, shown in FIG. 6B, for the pupil size of 3.5 mm in diameter with uncorrected astigmatism of CYL=⅝ D. Images with the best quality for acuity for different spherical aberration S₁=0, −0.26, −0.78, −1.04, −1.30 are identified and boxed.

From the simulated retinal images in FIG. 6B, we have a few findings. First, for a conventional aspherical lens that corrects eye's spherical aberration (S₁=0, first column in FIG. 6B), image blur makes it impossible to recognize a complete set of acuity letters for 20/20 (the 2^{nd} smallest letters in the chart, fourth row from top) or even 20/25. Poor acuity of 20/40 or worse plus image distortion are observed when the uncorrected CYL=⅝ D is mixed with SPH error of ±0.25 D and ±0.5 D. Second, for a spherical lens that leaves eye's spherical aberration uncorrected (S₁=−0.26, second column in FIG. 6A), image distortion is seen in all five focus settings. The best quality of vision is found with a focus offset +0.25 D with image distortion of all acuity letters between 20/16 and 20/30. All images with +/−0.25 D and +/−0.5 D are blurred with difficulty for recognizing letters of 20/40 or worse. It is likely that the best corrected vision will be worse than 20/20, and quality of corrected vision is poor due to image distortion caused by phase shift in the phase transfer function. Third, for a new kind of wavefront aspherical lens that induces more spherical aberration into eye's central pupil (S₁ is more than 0.52 microns in magnitude, S₁=−0.78, −1.04, and −1.30), we see improved vision in three aspects: 1) improved best corrected visual acuity to 20/20 or even 20/16, 2) improved quality of vision by eliminating distortion, 3) more tolerance in errors in focus correction.

Similarly, it is also found in FIG. 6C and FIG. 6D that a wavefront aspherical lens that induces positive spherical aberration for S₁=0.78, 1.04, and 1.30 microns for a 3.5 mm pupil also improves acuity, quality of vision, and focus tolerance if eye's uncorrected astigmatism is ⅝ D.

Contrary to the universal belief that inducing spherical aberration into an eye would degrade the best corrected vision, we have shown for the first time that inducing spherical aberration into an eye's central pupil can improve acuity and quality of vision if uncorrected astigmatism of ⅝ D is left uncorrected by an ophthalmic lens (contact lenses/ICLs/IOLs), and the best corrected acuity can be improved from 20/40 and 20/30 to 20/20 or better.

Having shown that inducing spherical aberration into eye's central pupil by a wavefront-engineered monofocal lens can mitigate uncorrected astigmatism of ⅝ D for improved best corrected acuity, we would also like to see the impact of induced spherical aberration for eyes with less uncorrected astigmatism such as CYL=⅜ D or even with a perfect correction of astigmatism CYL=0D.

FIG. 6E shows eye's point-spread functions for a hypothetical human eye for a pupil size of 3.5 mm in diameter with CYL=⅜ D, while the same six cases of spherical aberration in the eye are considered: 1) S₁=0 (first column from the left) if eye's spherical aberration is corrected by a conventional aspherical lens, 2) S₁=−0.26 (second column from the left) if the eye's spherical aberration is left unchanged by a conventional spherical lens, 3) S₁=−0.52, −0.78, −1.04, and −1.3 if additional spherical aberration into an eye by a wavefront aspherical lens. We also consider eyes with different amounts of focus errors: SPH=−0.5 D, −0.25 D, 0D, 0.25 D, 0.5 D.

Similar to the results in FIG. 6A and FIG. 6C, it is observed that inducing spherical aberration has the same effect of mitigating astigmatism of CYL=⅜ D in FIG. 6E: 1) eye's point spread function is large in size when the eye's spherical aberration is completely corrected (S₁=0 in the first column from left) or unchanged (S₁=−0.26 in the 2nd column from left). The eye's point spread function is reduced in size when more spherical aberration is induced for S₁=−0.78, −1.04, and −1.3.

From the point spread functions in FIG. 6E, we calculated the retinal image of an acuity chart for the hypothetical human eye, shown in FIG. 6F, for a pupil size of 3.5 mm in diameter. Images with the best quality for acuity for S₁=0, =−0.26, −0.78, −1.04, −1.30 are identified and boxed.

For astigmatism of ⅜ D left uncorrected by a monofocal lens, we have similar findings in FIG. 6F (CYL=⅜ D) and in FIG. 6B (CYL=⅝ D) and FIG. 6D (CYL=⅝ D): a new kind of wavefront aspherical lens, that induces more spherical aberration into eye's central pupil (S₁=−0.78, −1.04, and −1.30), will improve quality of vision beyond conventional aspherical lenses (S₁=0) and conventional spherical lens (S₁=−0.26) in three aspects: 1) improved best corrected visual acuity beyond 20/16, 2) eliminating distortion due to phase shift in the phase transfer function, 3) more tolerance in errors in focus correction.

For a hypothetical eye with either no astigmatism or astigmatism that is completely corrected, FIG. 6G shows the eye's point-spread functions for a pupil size of 3.5 mm in diameter. The eye with the most compact point spread function is found: 1) at one focus setting of SPH=0 for S₁=0, 2) at two focus settings of SPH=0, 0.25 for S₁=−0.26, 3) at two focus settings of SPH=0.25 D, 0.50 D for S₁=−0.52 and S=−1.04, at three focus setting of SPH=0, 0.25, 0.50 D for S₁=−0.78 and S₁=−1.3.

Looking at the simulated acuity chart in FIG. 6H, we can conclude that, in a rare case (about 1/20), even when an eye has a perfect correction for astigmatism (CYL=0) by a monofocal/toric lens, the new kind of wavefront aspherical lens that induces more spherical aberration into eye's central pupil (S₁=−0.78, −1.04, and −1.30) still improves vision correction beyond conventional aspherical lenses (S₁=0) and conventional spherical lens (S₁=−0.26) by 1) increasing tolerance for the error in focus power while achieving the same best acuity beyond 20/16 or better with very little reduction in contrast, 2) eliminating distortion due to phase shift in the phase transfer function caused by a small error in focus correction.

It is also noticed that adding a focus offset beyond the induced spherical aberration in the central pupil will achieve the best quality.

In addition to the conventional baseline Diopter power for a spherocylindrical correction, the wavefront-engineered monofocal lens intentionally makes the lens imperfect according to the conventional definition. The wavefront errors introduced in the central optical section of the wavefront-engineered monofocal lens can be expressed as,

$$W ( ρ , φ ) = {S}_{1} * {( ρ \text{/} {r}_{0} )}^{4} - 0.5 * Φ * {ρ}^{2} ,$$

where r₀=0.5*D₀ is a radius of the central aspherical section, ρ is a polar radius in a pupil plane, which has a value between 0 and r₀, ϕ is a focus offset in Diopter, and S₁ is the total spherical aberration induced into the wavefront-engineered monofocal lens.
1 D. Mitigation of Coma by Inducing Spherical Aberration into Eye's Central Pupil

Coma in eyes degrades quality of vision. Wavefront correction of coma and high-order aberrations was demonstrated using adaptive optics by J Liang, D R Williams, D T Miller, published in “Supernormal vision and high-resolution retinal imaging through adaptive optics” in Journal of the Optical Society of America A Vol. 14, Issue 11, pp. 2884-2892 (1997). Wavefront correction of high-order aberrations was also proposed in U.S. Pat. No. 5,777,719.

Effective correction of coma in eyes has not been effectively demonstrated for normal eyes in eyeglasses, contact lenses, and IOLs for many reasons. First, coma in each eye must be individually measured. Second, coma-correcting lenses (eyeglasses, contact lenses, IOLs) must be custom made. Third, precise registration in lens position and orientation of the coma-correcting lenses to the eye must be achieved for eyeglasses, contact lenses, IOLs to coma in an eye.

In one aspect of the present invention, we show therapeutic treatments for coma by inducing additional spherical aberration in the central pupil of eye in FIG. 6I and FIG. 6J.

FIG. 6I shows calculated retinal images of an acuity chart for a hypothetical eye with only coma left uncorrected by a conventional monofocal lens (left column) and by a wavefront-engineered monofocal lens that induces spherical aberration (S₁) of −0.78 microns for a pupil size of 3.5 mm (right column). Coma in the simulated eye is measured by a Zernike polynomial with a Zernike coefficient of 1.0 micron for a 6 mm pupil. Annoying image blurs and image distortion caused by coma in the eye (left column) is effectively eliminated by the wavefront lenses (right column).

FIG. 6J shows simulation results with a Zernike coefficient for coma increased from 1.0 micron to 1.5 microns for a 6 mm pupil. Effectiveness of using wavefront lenses for mitigation of significant coma is still evident.

### 1E. Wavefront-Engineered Monofocal/Toric Contact Lenses, ICLs, IOLs

U.S. Pat. No. 8,529,559 B2 and US patent application # 2011/0029073 A1 disclosed methods and devices of inducing spherical aberration into eye's central pupil for presbyopia corrections. Before our discoveries in the present invention, inducing more spherical aberration into eye by correction lenses has been universally believed to have negative effect in image contrast. We have shown in the present invention that, in addition to increasing depth of focus, inducing spherical aberration in the central pupil of an eye is also effective for improving quality of vision corrections: improved Best Corrected Visual Acuity (BCVA) and mitigating uncorrected astigmatism, coma, focus errors left by a spherocylindrical correction.

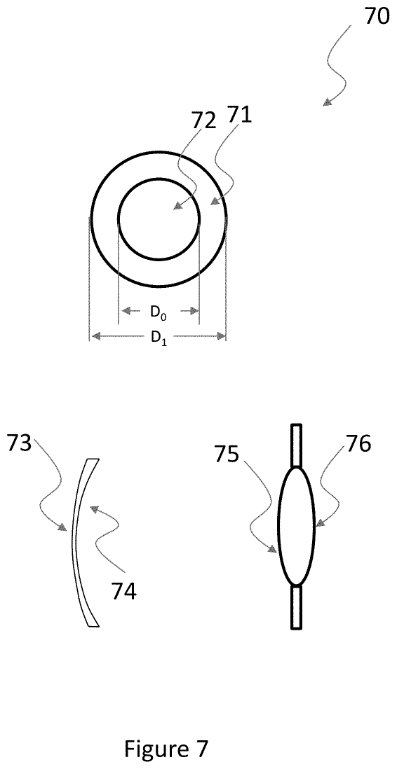

We disclose a wavefront-engineered monofocal lens for an eye in FIG. 7. The lens 70 is configured as an IOL (75,76) or a contact lens (73,74) or an ICL, and it comprises: 1) a baseline Diopter power extending across an optical section of the lens (71+72) for the correction of far vision defects, and the optical section having a diameter D₁ between 5 mm and 8 mm and the correction of far vision defects including at least a focus error and/or a cylinder error, 2) at least a central aspherical section in the center of the lens (72) that uses at least one aspheric surface (73 or 74, 75 or 76) to induce spherical aberration into eye's central pupil. The central aspherical section has a diameter D₀ between 2.5 mm and 4.5 mm. The baseline Diopter power is normally specified as a spherocylindrical correction. The wavefront errors introduced in the aspherical section provides treatments for (or mitigation to) residual refractive errors left uncorrected in the eye by the baseline Diopter power for far vision defects. The uncorrected refractive errors in the eye left by the lens include astigmatism, focus errors (myopic or hyperopic powers), coma, and other higher order aberrations that are significant in degrading vision at least in the central pupil of an eye. The uncorrected (residual) refractive errors can further include a presbyopia power less than +1.0 D. If the presbyopia power is more than 1.0 D such as 2 D in U.S. Pat. No. 8,529,559 B2 and US patent application # 2011/0029073 A1, corrected vision suffers from significant loss in image contrast for far vision for a pupil size around 3.5 mm, leading to worse than 20/20 at far distances. The wavefront-engineered monofocal lens can be adapted as a contact lens, an Intraocular Lenses (IOL), or an Accommodating Intraocular Lenses (AIOL), an Implantable Contact Lenses (ICL), a phakic IOL.

In one embodiment, the central aspherical section is further configured to induce an additional focus offset between −0.75 D and +1.25 D on top of the baseline Diopter power.

In another embodiment, the induced spherical aberration in the central aspherical section can be expressed as a wavefront error of S₁*(ρ/ρ₀)⁴, and ρ₀=0.5*D₀ is a radius of the central aspherical section, ρ is a polar radius in a pupil plane and having a value between 0 and ρ₀. ρ₀ is between 1.25 mm and 2.25 mm.

In yet another embodiment, S₁ is positive and greater than 0.78*(D₀/3.5)⁴ in magnitude or negative and more than 0.26*(D₀/3.5)⁴ in magnitude. D₀ is a diameter of the aspherical section. The combined spherical aberration from the eye under the correction and the wavefront-engineered monofocal lens is more than two times as much as the statistical mean of eye's spherical aberration in normal human eyes in magnitude.

In addition to the conventional baseline Diopter power for a spherocylindrical correction, our invention of the wavefront-engineered monofocal lenses intentionally makes the monofocal lens imperfect according to the conventional definition. The wavefront errors introduced in the central optical section of the wavefront-engineered monofocal lens can be expressed as,

$$W ( ρ , φ ) = {S}_{1} * {( ρ \text{/} {r}_{0} )}^{4} - 0.5 * Φ * {ρ}^{2} ,$$

where r₀=0.5*D₀ is a radius of the central aspherical section, ρ is a polar radius in a pupil plane, which has a value between 0 and r₀, ϕ is a focus offset in Diopter, and S₁ is the total spherical aberration induced into the wavefront-engineered monofocal lens.

TABLE 2A
Parameters for an exemplary wavefront-engineered monofocal lens

| r₀ (mm) | S₁ (microns) | Φ(Diopters) |
|---|---|---|
| 2.0 | 4.69 | 0.65 |

In one exemplary embodiment for further increased tolerance for uncorrected astigmatism as well as to extend depth of focus, Table 2A lists the parameters for an exemplary wavefront design.

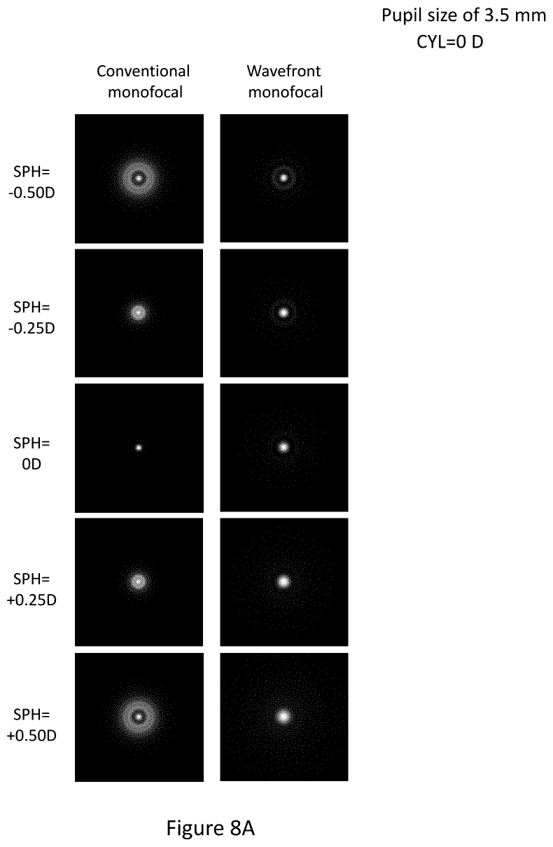

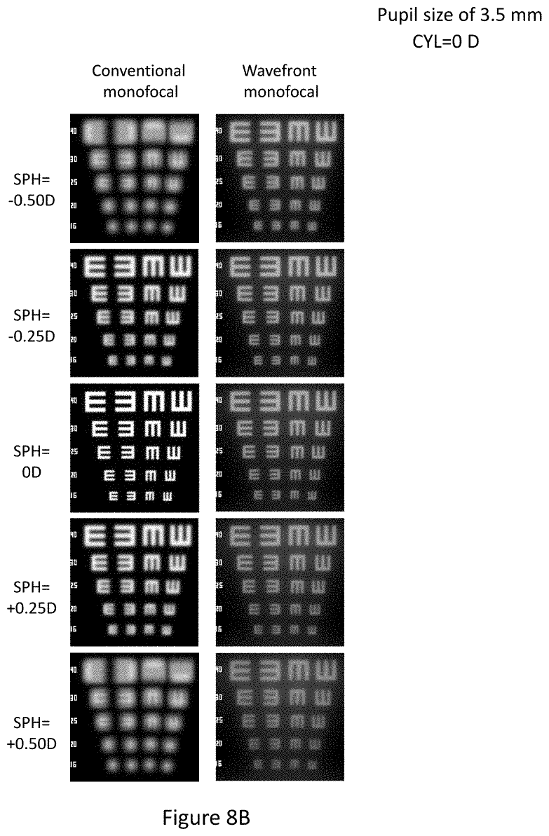

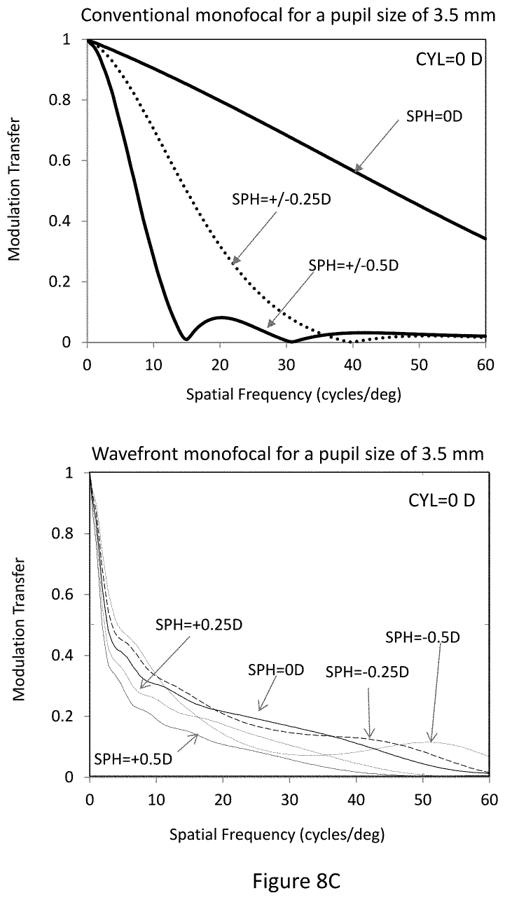

FIG. 8A shows point spread functions of a hypothetical eye for a pupil diameter of 3.5 mm with a conventional monofocal lens (left column) in comparison to the exemplary wavefront-engineered monofocal lens (right column) with induced spherical aberration and focus offset in Table 2A. The hypothetical eye is considered to have no astigmatism (CYL=0), and a focus error (SPH) between −0.5 D and +0.5 D is left uncorrected by the monofocal lens. It is seen that, except for the perfect spherical correction when SPH=0, the point-spread function of the wavefront-engineered monofocal lens (right column) is more compact than that of the conventional monofocal lens (left column) in all the cases when SPH=−0.5 D, −0.25 D, 0.25 D and 0.5 D.

FIG. 8B shows the calculated retinal images from the point spread functions for the cases in FIG. 8A for the conventional monofocal lens (left column) in comparison to the wavefront-engineered monofocal lens (right column). In addition, we show the calculated Modulation Transfer Functions (MTF) from the point spread functions for the cases in FIG. 8A for the conventional monofocal lens (top) and the wavefront-engineered monofocal lens in the exemplary design (bottom) in FIG. 8C.

For a perfect correction with SPH (SPH=0) and CYL (CYL=0), which is extremly rare (e.g., less than 1 in 20 eyes), as expected, inducing spherical aberration by the wavefront lens significantly reduces contrast of retinal image for all frequencies as seen in the images (middle row in FIG. 8B) and in MTF in FIG. 8C. Retinal contrast for the wavefront lens is reduced from 68% to 16% at 30 c/deg for 20/20, from 59% to 12% for 37.5 c/deg for 20/16, and from 47% to 5% at 48 c/deg for 20/12. It must be mentioned that this ideal case of SPH=0 and CYL=0 has liitle or no practical impact because a perfect focus correction for both SPH and CYL is extremly rare and retinal contrast in real eyes are further degraded by third-order Zernike aberrations such as coma (see “Aberrations and retinal image quality of the normal human eye” publised in Journal of the Optical Society of America A Vol. 14, Issue 11, pp. 2873-2883 (1997) by J Liang and D R Williams) . A formula for the mean human optcial modulation trasfer function as a function for pupil size was published by A B Watson in Journal of Vision, 13 (6):18, pp. 1-11 (2013).

SPH is normally not perfectly corrected due to 1) myopic power of −0.25 D between far vision at infinity and far vision at 4 meters in vision test, 2) errors in manufactured lens or errors in the eye refraction. For SPH=−0.25 D and SPH=0.25, the hypothetical eye cannot recognize any letter of 20/16 acuity or smaller letters with the conventional monofocal lens with a perfect correction of both SPH and CYL, shown in FIG. 8B, because the retinal contrast is only about 1.2% at spatial frequency of 37.5 cycles/degree and 2.1% for 20/16 acuity and 48 cycles/degree for 20/12.5 acuity, as shown in FIG. 8C. MTF of the conventional monofocal lens is less than 2.5% in the entire spatial frequency range from 36 cycles/degree and 48 cycles/degree, leading to a limitation of best corrected acuity below 20/16.

This is completely different for our wavefront-engineered monofocal lens. The wavefront design improves retinal contrast from less than 1.2% to 14% for SPH=−0.25 D and to 5% for SPH=0.25 for at 37.5 cycles/degree for 20/16 acuity, and improves retinal contrast from 2.1% to 11% for SPH=−0.25 D at 48 cycles/degree for 20/12.5 acuity. Thus, the wavefront-engineered monofocal lens enables the hypothetical eyes to achieve the best corrected visual acuity of 20/16, shown in FIG. 8B, or even 20/12.5 for SPH=−0.25 D. It is also observed, when compared to the conventional monofocal lens, our wavefront-engineered monofocal lens pays a small price of slightly reduced retinal contrast at the low frequencies such as 15 cycles/degree for 20/40 acuity and 20 cycles/degree for 20/30 acuity, and gains better vision for improving image contrast and clarity for spatial frequency higher than 24 cycles/degree (20/25 acuity).

For SPH=−0.5 D and SPH=0.5 D, the hypothetical eye with the conventional monofocal lens cannot see letters of 20/40 and 20/20, shown in FIG. 8B, because the retinal contrast is nearly zero at 15 cycle/degree and 30 cycles/degree, shown in FIG. 8C. It is also noticed that letters of 20/30 and 20/25 are distorted, shown in FIG. 8B, due to a phase reversal in the Phase Transfer Function (PTF) between 15 cycles/degree and 31 cycles/deg. A phase reversal in PTF causes position dispalcedment of the conresponsding spatial frequency by a half cycle. On the contrary, the wavefront-engineered monofocal lens enables the hypothetical eye to see all acuity letters between 20/40 and 20/16 without any distortion, shown in FIG. 8B. For SPH=−0.5 D, the wavefront-engineered monofocal lens would even enable one to see 20/12 letters with a retinal contrast of 11% at 48 cycles/degree. Debluring the degraded retinal images of the conventional monofocal lens by the wavefront-engineered monofocal lenses is achieved by 1) eliminating nearl 100% loss of retinal contrast in eye's MTF between 15 cycles/degree and 40 cycles/degree, 2) eliminaing the phase reversal in eye's PTF of conventional lens.

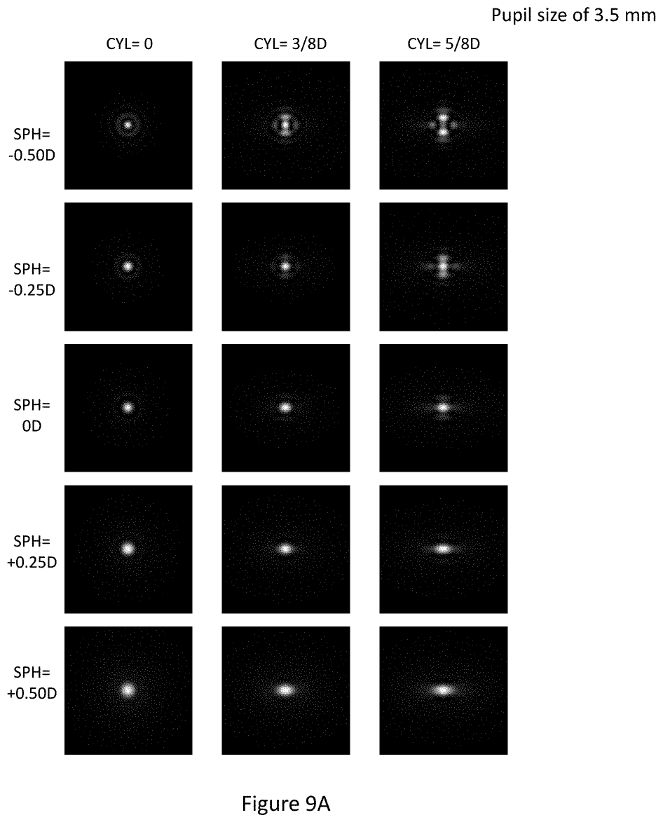

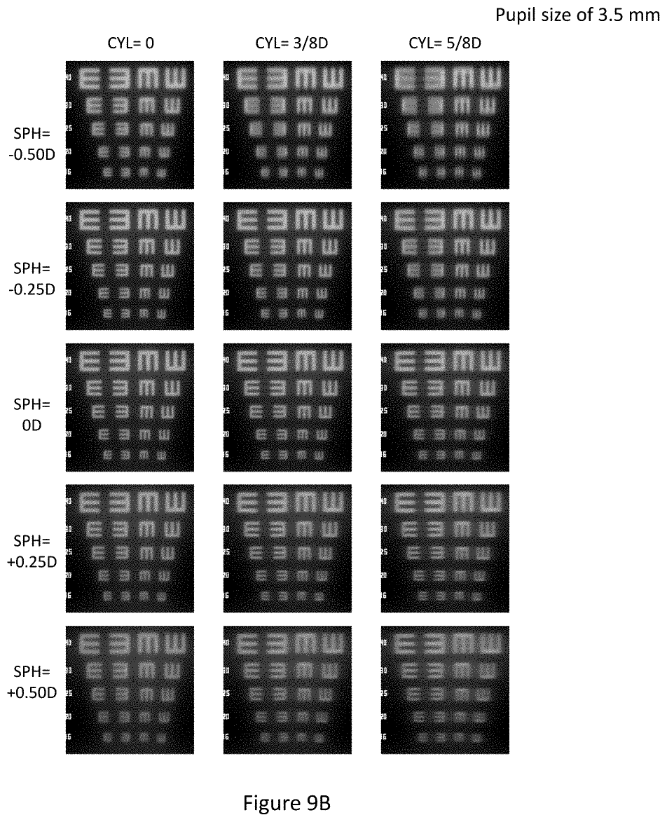

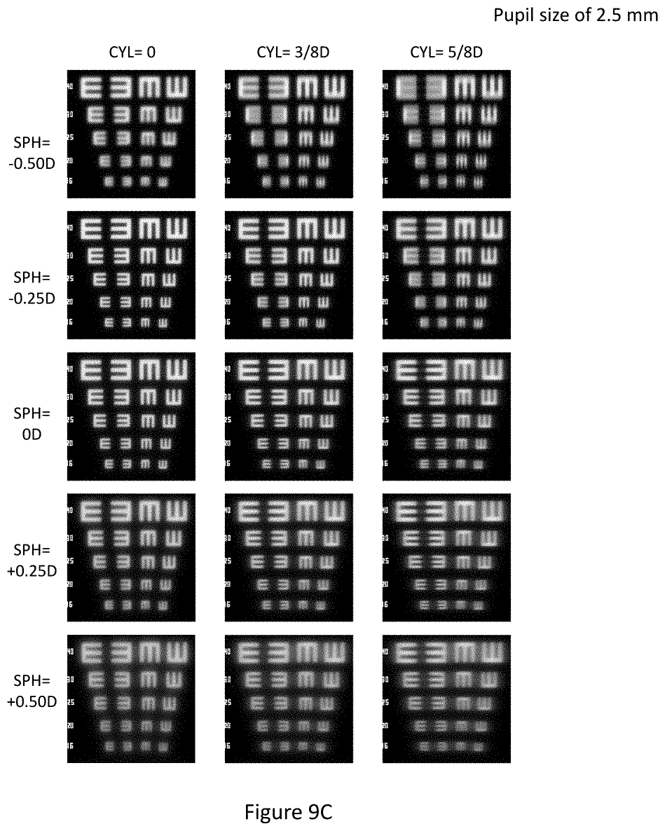

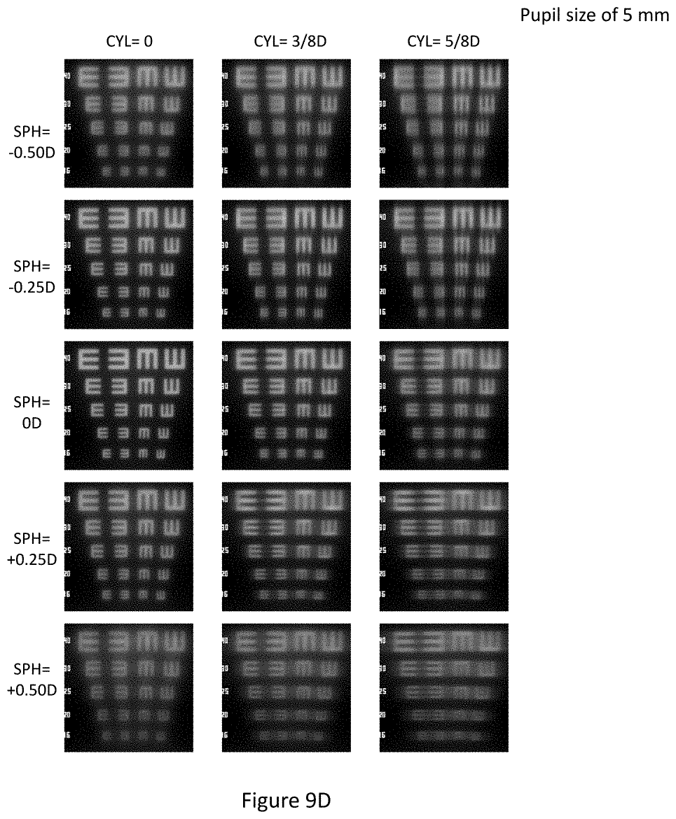

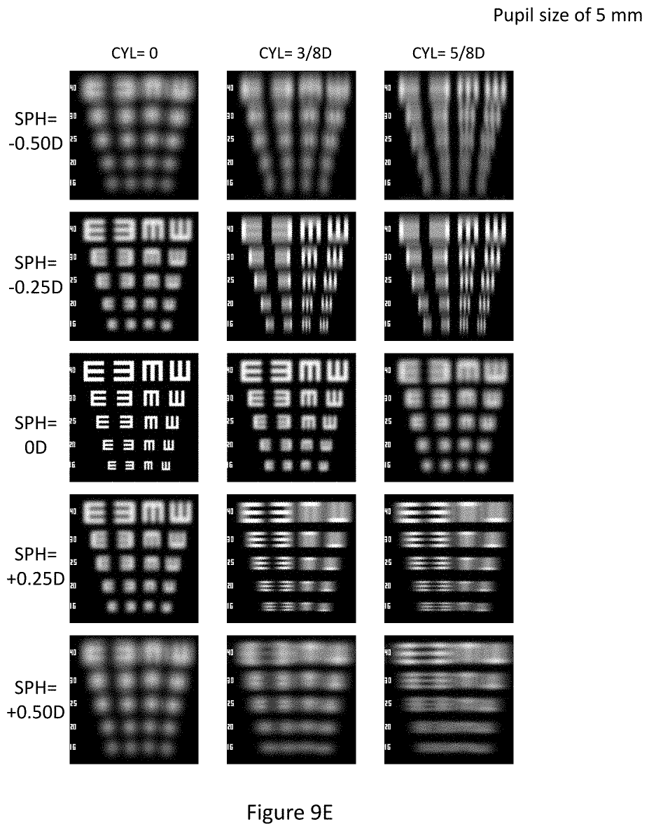

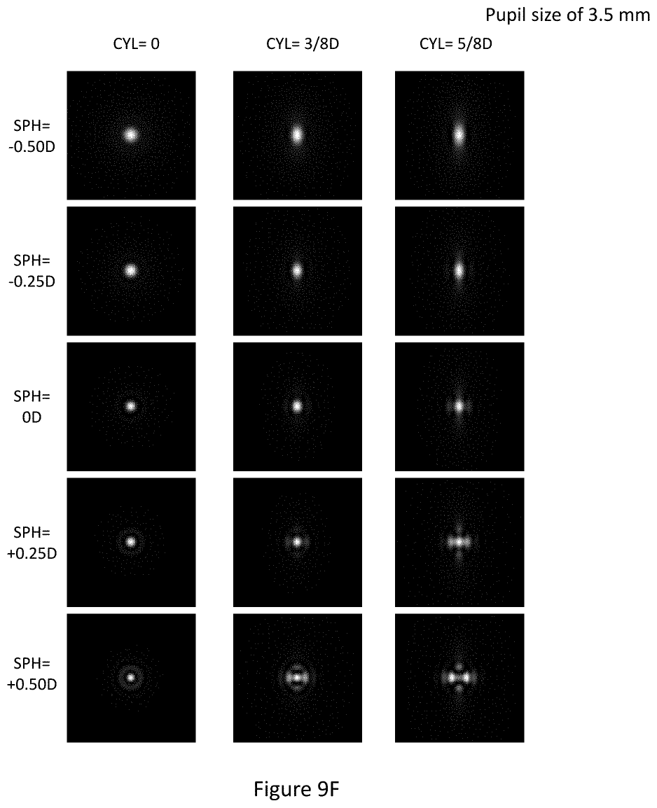

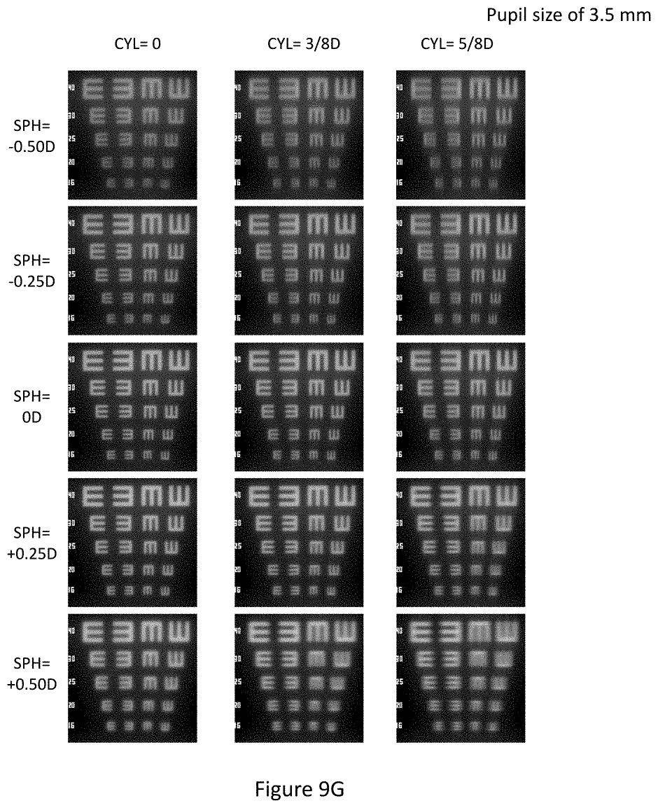

In order to study correction of residual astigmatism, focus errors, and its pupil size dependency of the exemplary wavefront-engineered monofocal lens, specified in Table 2A, we provide optical simulation in FIG. 9A through FIG. 9D.

FIG. 9A shows calculated point-spread functions of a hypothetical human eye for a pupil size of 3.5 mm in diameter for the exemplary wavefront-engineered monofocal lens in Table 2A. We also calculated retinal images of human eyes for a tumbling E chart for different pupil sizes in FIG. 9B for a 3.5 mm pupil size (indoor and acuity test).

Striking differences in three aspects are observed when the retinal images are compared between the conventional monofocal lens in FIG. 5B and the wavefront lens in FIG. 9B under the identical condition of pupil size of 3.5 mm.

Firstly, unlike the conventional monofocal lens in FIG. 5B, de-astigmatism is seen with the wavefront-engineered monofocal lens in FIG. 9B. There is little to no difference in calculated retinal images under different values of astigmatism (CYL) with the same focus error (SPH) in FIG. 9B.

Secondly, the wavefront-engineered monofocal lens provides exceptional acuity: 1) 20/16 acuity can be obtained independent of residual astigmatism in the eye with a tolerance of focus error of at least ±0.25 D, 2) acuity of 20/20 is achieved for focus error of ±0.5 D with a residual astigmatism up to ⅝ D.

Thirdly, quality of vision is improved with the wavefront-engineered monofocal lens because it eliminates image distortion of conventional lenses caused by residual focus errors or/and residual cylinder error shown in FIG. 5B. In Fourier Optics, image blur of an optical system is characterized by 1) losses in image contrast for different spatial frequencies of the object, which is measured by a Modulation Transfer Function (MTF), 2) phase shifts or phase reversals between different spatial frequencies of the object, which is measured by a Phase Transfer Function (PTF). A phase reversal for a given spatial frequency leads to a position shift by a half cycle for the special frequency in the retinal image. When the displaced spatial frequencies by a half cycle are summarized with the non-displaced spatial frequencies of the object, the final retinal image is not only blurred but also distorted, and this makes the letters distorted and uncomfortable for people to view.

We can conclude that the wavefront-engineered monofocal lens will improve vision correction for most normal eyes, but may result in reduced acuity or contrast for a small population (e.g., 1 in 20) with monofocal best corrected acuity of 20/10.

Modern cameras use autofocus to correct the focus error dynamically, and employ aspherical lenses as well as multiple lens elements to correct spherical aberration, astigmatism, and coma. Spherical aberration by its definition degrades image quality of an optical system, and this is certainly true for camera lenses as well as for human eyes with a large pupil size at night. Using spherical aberration to improve visual acuity and quality of vision is counterintuitive, but it makes perfect sense when we consider the imperfect nature of ophthalmic corrections with state of the art IOLs and contact lenses, shown in FIG. 5A and FIG. 5B.

Quality of an ophthalmic lens for an eye must consider vision for different pupil diameters: e.g., 2.5 mm for outdoor and daylight and 5 mm for night vision. FIG. 9C and FIG. 9D show the calculated retinal images for the same hypothetical eye for a pupil diameter reduced to 2.5 mm or increased to 5 mm, respectively.

Compared to the calculated retinal images in FIG. 9B for a 3.5 mm pupil, retinal images in FIG. 9C for a 2.5 mm pupil have much better contrast and legibility for the acuity letters for each combination of astigmatism and focus error.

Simulation of retinal point-spread functions and retinal images for night vision is difficult because we need to consider the eye's high-order aberrations at night that are different from eye to eye. For simplicity, we assume the uncorrected astigmatism and focus error left by the monofocal lenses are still more significant than the eye's high-order aberrations, which is reasonable for astigmatism of ⅜ D and ⅝ D, and/or a focus error of +/−0.25 D and +/−0.5 D.

FIG. 9D and FIG. 9E show the calculated retinal images of a hypothetical eye for a pupil size of 5 mm in diameter for the exemplary wavefront-engineered monofocal lenses (FIG. 9D) and a conventional monofocal lens (FIG. 9E), respectively. The wavefront errors of the wavefront-engineered monofocal lens do not extend beyond a 4 mm pupil diameter but the uncorrected astigmatism and focus errors do extend to the entire 5 mm pupil. It is evident that except for the rare case for SPH=0 and CYL=0, night vision performance for a 5 mm pupil of the exemplary wavefront-engineered monofocal lens is significantly better than that of a conventional monofocal lens for quality of vision and acuity. The effect at night in comparing FIG. 9D (wavefront monofocal) and FIG. 9E (conventional monofocal) looks more dramatic than the comparison in a pupil diameter of 3.5 mm in comparing FIG. 9B (wavefront monofocal) and FIG. 5B (conventional monofocal).

Therefore, we can conclude that, when uncorrected astigmatism, coma, and focus errors left by conventional monofocal lenses are considered in human eyes, spherical aberration in the central pupil is no longer a negative factor in designing ophthalmic lenses and eyepieces in vision devices.

In another exemplary embodiment of wavefront-engineered monofocal lenses, the wavefront errors introduced into the aspherical section are a negative spherical aberration (S₁<0) and a negative focus offset. Table 2B lists the parameters for the second exemplary wavefront-engineered monofocal lens.

FIG. 9F shows the calculated retinal image of a point source, point-spread function, of a hypothetical human eye with a pupil size of 3.5 mm in diameter for the second exemplary wavefront-engineered monofocal lens. From the calculated point-spread function in FIG. 9F, we also calculated the retinal images for a tumbling E chart, which is shown in FIG. 9G.

TABLE 2B
Parameters for an exemplary wavefront-engineered monofocal lens

| r₀ (mm) | S₁ (microns) | Φ(Diopters) |
|---|---|---|
| 1.75 | −2.75 | −0.65 |

The second exemplary wavefront-engineered monofocal lens in Table 2B, which uses a negative spherical aberration (S₁<0) and a negative focus offset, shares similar advantages with the first wavefront-engineered monofocal lens in Table 2A that uses a positive spherical aberration (S₁>0) and a positive focus offset. We also notice one clear difference between them: the second exemplary wavefront-engineered monofocal lens (Table 2B) has better quality of vision for positive focus errors SPH=0.25 D and 0.50 D while the first exemplary wavefront-engineered monofocal lens (Table 2A) has better quality of vision for positive focus errors SPH=0.25 D and 0.50 D.

In one embodiment of the wavefront-engineered monofocal lens, the induced total spherical aberration is negative (S₁<0) and the induced focus offset ϕ is negative and less than 0.75 D in magnitude (ϕ>−0.75 D). The induced negative spherical aberration (S₁) is between −0.71 microns and −7.51 microns in the central aspherical section, which is scaled for a pupil diameter between 2.5 mm and 4.5 mm according to Table 2C, showing spherical aberration (S₁) induced in a pupil with a different radius for the aspherical zone r₀ between 1.25 mm and 2.25 mm.

In another embodiment, the induced total spherical aberration is positive (S₁>0) and the induced focus offset ϕ is positive and less than 0.75 D in magnitude (ϕ<0.75 D). The induced positive spherical aberration (S₁) is between 0.71 microns and 7.51 microns in the central aspherical section, which is scaled for a pupil diameter between 2.5 mm and 4.5 mm according to Table 2C, showing spherical aberration (S₁) induced in a pupil with a different radius for the aspherical zone r₀ between 1.25 mm and 2.25 mm.

TABLE 2C
Parameters for a wavefront-engineered monofocal lens

| Radius of aspheric zone | ρ₀ (mm) | 1.25 | 1.75 | 2.25 |
|---|---|---|---|---|
| Spherical | S₁ (μm) | −0.71 | −2.75 | −7.51 |
| aberration = −2.75*(ρ₀/1.75)⁴ | | | | |
| Spherical | S₁ (μm) | 0.71 | 2.75 | 7.51 |
| aberration = 4.69*(ρ₀/2)⁴ | | | | |

In still another embodiment, the induced spherical aberration further includes a generalized spherical aberration that is characterized as a wavefront error of ρⁿ, and n is an integer equal to or greater than 3. The wavefront error by a generalized spherical aberration can be represented by a generalized polynomial ϕ(ρ)=c₃ ρ³+c₄ ρ⁴+c₅ ρ⁵+c₆ ρ⁶ and more. In one case, the induced spherical aberration further includes higher order spherical aberration that is characterized as a wavefront error of ρⁿ, where n is an even integer and larger than 4.

TABLE 2D
Exemplary designs of wavefront-engineered monofocal lenses

| | Parameters | WFM-CL1 | WF-EDOF M1 | WFM-CL2 | WF-EDOF M2 |
|---|---|---|---|---|---|
| Central | Radius R₁ (mm) | 1.75 | 1.75 | 1.75 | 1.75 |
| Aspherical | S.A. S₁ (Microns) | −1.2 | −2.75 | 1.2 | 2.75 |
| Zone | Focus offset Φ₁ | −0.37 | −0.15 | 0.25 | 0.85 |
| | (Diopter) | | | | |
| Annular | Radius R₂ (mm) | 3.0 | 3.0 | 3.0 | 3.0 |
| Aspherical | S.A. S₂ (Microns) | 0 | 0 | 0 | 0 |
| Zone | Focus offset Φ₂ | 0 | 0 | 0 | 0 |
| | (Diopter) | | | | |

Additional embodiments of wavefront-engineered monofocal lenses are provided in Table 2 D. WFM-CL1 and WFM-CL2 are optimized for wavefront contact lenses for patients without presbyopia. WF-EDOF M1 and WF-EDOF M2 are optimized for wavefront EDOF monofocal lenses for patients with presbyopia correction, and they can be adapted for contact lenses, IOLs, accommodation IOLs. Table 2E lists the induced spherical aberration in the aspherical central zone.

TABLE 2E
Positive spherical aberration in the central zone

| | Diameter of the central | | | | |
|---|---|---|---|---|---|
| | aspherical section | D₀ (mm) | 2.5 | 3.5 | 4.5 |
| WFM-CL1 | Spherical aberration in the | S₁ (μm) | −0.31 | −1.20 | −3.28 |
| | central section = −1.2* (D₀/3.5)⁴ | | | | |
| WF-EDOF M1 | Spherical aberration in the | S₁ (μm) | −0.72 | −2.75 | −7.51 |
| | central section = −2.75* | | | | |
| | (D₀/3.5)⁴ | | | | |
| WFM-CL2 | Spherical aberration in the | S₁ (μm) | 0.31 | 1.20 | 3.28 |
| | central section = 1.2* (D₀/3.5)⁴ | | | | |
| WF-EDOF M2 | Spherical aberration in the | S₁ (μm) | 0.72 | 2.75 | 7.51 |
| | central section = 2.75* (D₀/3.5)⁴ | | | | |

All these designs (WFM-CL1, WFM-CL2, WF-EDOF M1, WF-EDOF M1) as well as the designs in Table 2A and Table 2B can be used for Implantable Contact Lenses (ICLs). ICLs share similar problems in limited selection of lenses (SPH or CYL), errors in cylindrical AXIS, errors in lens manufacturing, errors in refraction prescriptions, presbyopia of eyes. ICLs are less forgiving than contact lenses because they entail a surgical procedure.

In some embodiments, the wavefront-engineered monofocal lens is configured as a wavefront contact lens having a diameter between 9 mm and 16 mm, and it comprises a front surface and a back surface, and at least one of the front surface and the back surface is aspheric for inducing spherical aberrations in the central aspherical section.

In one embodiment, the wavefront contact lens is configured to have a focus offset is between +0.12 D and +1.2 D, and the induced spherical aberration in the central pupil is between 0.31 microns and 7.51 microns in the central aspherical zone with a diameter between 2.5 mm and 4.5 mm.

In another embodiment, the wavefront contact lens is configured to induced spherical aberration in the central pupil between −0.31 microns and −7.51 microns in the central aspherical zone with a diameter between 2.5 mm and 4.5 mm, and a focus offset is less than 0.5 D in magnitude.

In yet another embodiment, the wavefront contact lens is further configured such that the induced spherical aberration in the central aspherical section (S₁) is custom determined based on the measured spherical aberration and other higher order aberrations in an individual eye.

In still another embodiment, the wavefront contact lens further includes correction of eye's high-order aberration for therapeutic treatments, wherein eye's high-order aberrations are aberrations except for astigmatism and focus error in an eye.

In another embodiment, the wavefront monofocal contact lens is further configured as a toric contact lens.

In yet another embodiment, the back surface of the contact lens is further configured to have an aspheric shape at a lens periphery for preventing lens rotation on the eye if the lens is a toric lens as well.

In some embodiments, the wavefront-engineered monofocal lens is configured as a wavefront monofocal intraocular lens (IOL) having a diameter of approximately 6 mm, e.g., between 5 mm and 7 mm, and it comprises a front surface and a back surface, and at least one of the front surface and the back surface is aspheric for inducing spherical aberrations in the aspherical section. The wavefront monofocal IOL further comprises a haptics section.

In one embodiment, the wavefront monofocal IOL is configured to have a negative focus offset less than 0.75 D in magnitude, the induced spherical aberration is between −0.31 microns and −7.5 microns in the central aspherical zone with a diameter between 2.5 mm and 4.5 mm.

In another embodiment, the wavefront monofocal IOL is configured to have a focus offset is between +0.25 D and +1.20 D and the induced spherical aberration is between 0.31 microns and 7.5 microns in the central aspherical zone with a diameter between 2.5 mm and 4.5 mm.

In yet another embodiment, the wavefront monofocal IOL is further configured as a toric IOL.

In still another embodiment, the wavefront monofocal IOL is configured as an accommodating IOL.

In some embodiments, the wavefront-engineered monofocal lenses (contact lenses, IOLs, and accommodating IOLs, ICLs) is further configured to include an aspherical section outside the central aspheric section for a) correcting spherical aberration in normal eyes at the pupil periphery, b) modifying spherical aberration at the pupil periphery in human eyes.

S. Plainis, D A Atchison and W N Charman studied four major brands of multifocal contact lenses and published their results in 2013 titled “Power Profiles of Multifocal Contact Lenses and Their Interpretation,” in Optometry and Vision Sciences, vol. 90, No. 10, pp1066-1077. Five contact lenses were found using aspherical surfaces to alter spherical aberration when they are placed on an eye: Air Optix-low, -med, -high from Alcon, and PureVision-Low, -High from Bausch & Lomb.

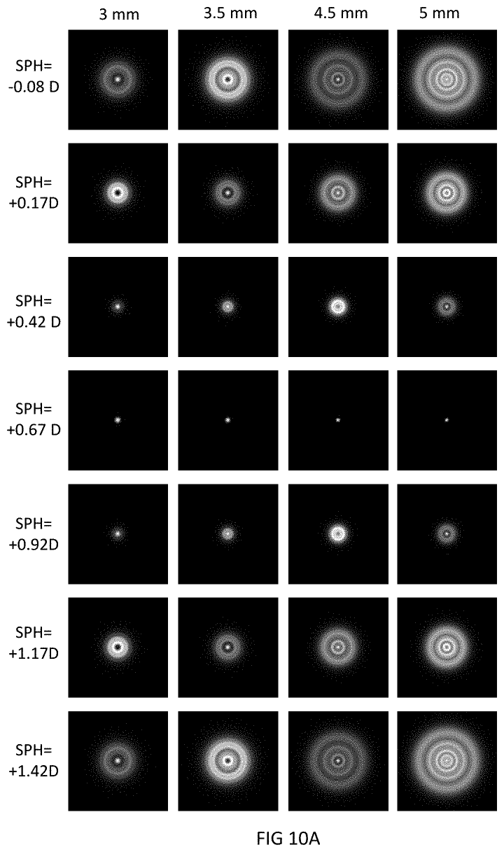

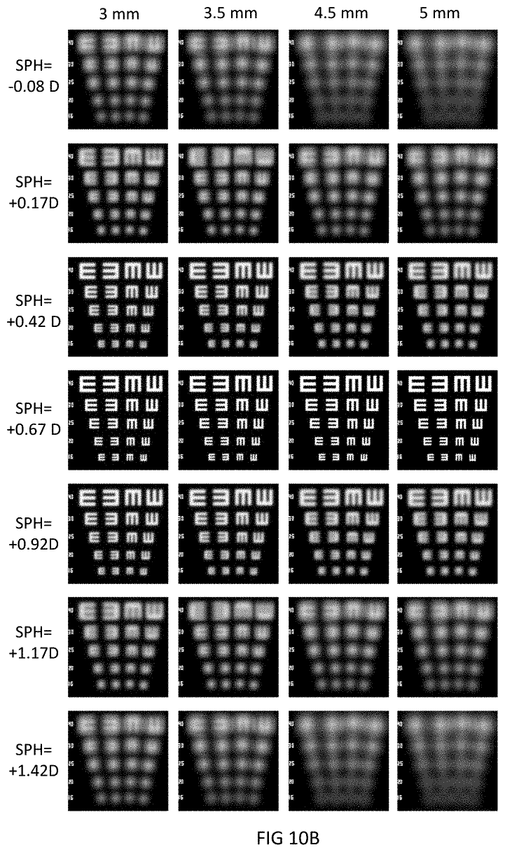

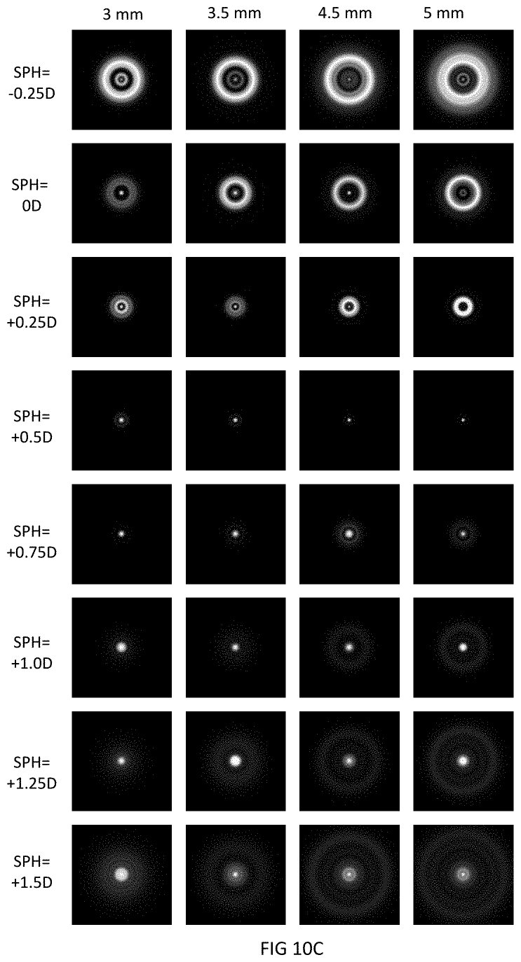

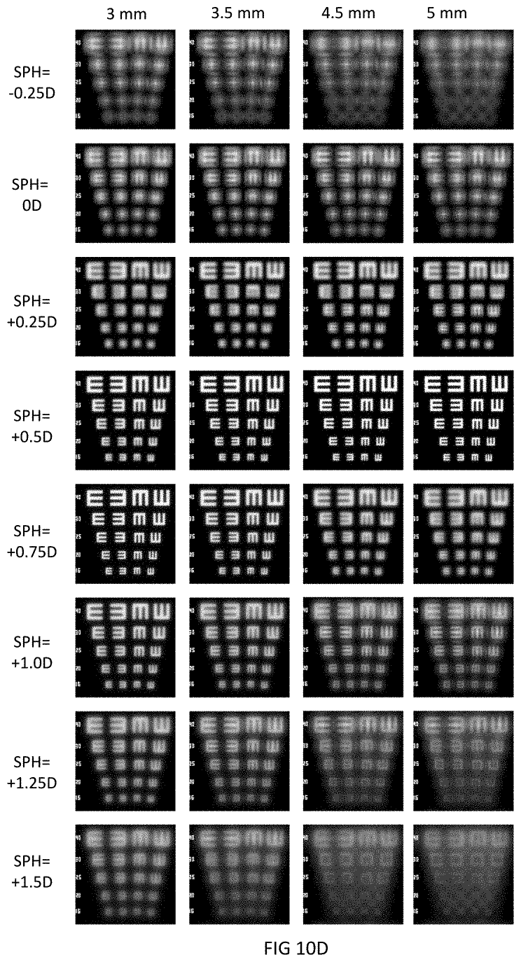

The “low” add PureVision from Bausch & Lomb and Air Optix from Alcon have a diopter profile of ϕ(η)=0.67−0.18 ρ² and ϕ(ρ)=0.54−0.15 ρ² with a diameter about 6 mm, respectively. They are essentially aspherical lenses for the correction of eye's mean spherical aberration in a normal population (0.112 ρ²), plus a positive focus offset of +0.67 D and 0.54 D beyond a baseline correction for a low presbyopia correction, respectively. Consumers, paying a premium for obtaining these so-called multifocal contact lens, could actually buy less expensive single-vision lenses with an offset SPH power of +0.50 D or +0.75 D in their prescription. FIG. 10A and FIG. 10B show calculated point-spread functions and calculated retinal images of an acuity chart for a PureVision-low lens from Bausch & Lomb. We have two conclusions. First, eye's best focus is shifted from a baseline correction (SPH=0) to additional SPH=+0.67 D in the entire lens as expected so that a low presbyopia between +0.5 D and +1.0 D is mitigated. At the same time, vision at far distances −0.08 D and +0.17 D is terribly blurred. Second, while offering a correction of eye's spherical aberration, these so-called multifocal contact lenses cannot be adapted as wavefront-engineered monofocal lenses described in the present invention because 1) they provides terrible vision at far distance as seen in FIGS. 10A and FIG. 10B, 2) they cannot provide mitigation for eye's uncorrected astigmatism which is shown for the case of S₁=0 in FIGS. 6A through FIG. 6H.

The “med” add Air Optix multifocal contact lens has a diopter profile of ϕ(ρ)=1.14−0.44 ρ² in the central pupil with a diameter of 2.8 mm. After corrections of eye's mean spherical aberration (0.112 ρ²) in a normal population and a baseline focus error of an individual eye, this lens leaves a diopter profile of ϕ′(ρ)=1.14−0.33 ρ². FIG. 10C and FIG. 10D show the calculated point-spread functions and retinal images of an acuity chart for an “Air Optix-med” lens, respectively. Best vision is set around +0.5 D with acceptable vision between +0.5 D and +1.25 D for indoor with a pupil size at 3 mm and at 3.5 mm. However, presbyopia corrections of “Air Optix-med” lenses also come with a heavy price for vision at far distance between −0.25 D and +0.25 D. Additionally, the “Air Optix med” lenses cannot be used for wavefront-engineered monofocal lenses as described in the present invention because far vision at 0 D and −0.25 D are terrible as seen in the FIGS. 10C/10D, and most people wearing Air Optix med lenses will not be able to pass a driving test to see 20/40 at around 6 meters based on the simulated results. Even if these lenses are prescribed for off-label uses, Air Optix med has the wrong combination of the focus offset and the induced negative spherical aberration.

The “high” add PureVision multifocal contact lens (Bausch & Lomb) and Air Optix multifocal contact lens (Alcon) have a diopter profile of ϕ(ρ)=1.93−0.50 ρ² and ϕ(ρ)=1.58−0.69 ρ² in the central pupil with a diameter of 2.4 mm and 2.8 mm, respectively. After corrections of eye's mean spherical aberration (0.112 ρ²) in a normal population and a baseline focus error of an individual eye, these lenses leave a diopter profile of ϕ′(ρ)=1.93−0.39 ρ² and ϕ′(ρ)=1.58−0.58 ρ², respectively. The structures of the “high” add PureVision and Air Optix multifocal lenses cannot be adapted for the wavefront monofocals described in the present invention because they degrade vision at far distance even more severely than Air Optix med lenses. Even if these lenses are prescribed for off-label use, they have the wrong combination of the focus offset and the induced negative spherical aberration.

### 2. Wavefront Extended Depth of Focus (EDOF) Bifocal Lenses

Bifocal lenses have two distinct optical powers, and they usually provide a first focus for vision at far distances and a second focus for a presbyopia correction.

Diffractive bifocals are available for IOLs with a Diopter separation between the two foci ranging from +1.75 D to 4.0 D. As mentioned earlier, problems with diffractive multifocal IOLs include 1) nighttime symptoms of halo and starburst due to simultaneous bifocal images, 2) spider-web type of night symptoms associated with diffractive structures, 3) ghost images of large objects at distance caused by defocused near focus, 4) poor vision between foci and image distortion due to focus error or astigmatism in the eye.

Because contact lenses cannot use the split-power design in spectacles or diffractive designs in IOLs due to a sharp diffractive surface, there is up to date no bifocal contact lenses that can offer presbyopia correction without severely degrading acuity at far distances. We showed that the so-called multifocal contact lenses (Air Optix from Alcon and PureVision from Bausch & Lomb) are monofocal lenses, and they cannot be qualified as bifocal lenses because patient's far vision has been severely compromised in FIG. 10A through FIG. 10D.

Inducing spherical aberrations of opposite sign in the central pupil was proposed in U.S. Pat. No. 8,529,559 B2 and US patent application # 2011/0029073 A1. In order to obtain a desired Depth of Focus (DoF) of 3 D for a presbyopia-correcting IOL, a focus offset of +4.0 D (being +1 D larger than the desired DOF of +3.0 D) was introduced in the central aspheric section. The design suffers from significant loss in retinal contrast for far distances for a pupil diameter of 3 mm or 3.5 mm (indoor and acuity test), a standardized diameter for IOL testing.

The Mini Well Ready IOL (Sifi S.p.A), designed based on inducing spherical aberrations of opposite sign into central pupil, solved the low contrast problem for far distances using a special configuration, and it provides an EDOF bifocal lens: a first focus for far distances with a high contrast PLUS a second extended depth of focus from +1.0 D to +2.5 D. However, the Mini Well Ready IOL also suffers from at least one drawback that the focus depth is 2.5 D, and much smaller than 3 D, required for reading at a close distance of 33 mm.

In one aspect of the present invention, we describe two EDOF bifocal lenses in Table 3A: one labeled as EDOF Bifocal 3 D for a high presbyopia correction of about 3 D, and the other labeled as EDOF Bifocal 1 D for a low presbyopia about +1.0 D. Differing from Mini Well Ready IOLs that has an extended depth of focus for the near distances (see “A New Extended Depth of Focus Intraocular Lens Based on Spherical Aberration” in J Refract Surg. 2017;33(6):389-394 by R Bellucci and MC Curatolo), our EDOF Bifocal lenses have an extended depth of focus for the far distance, which improve chances of achieving best corrected vision of 20/20 for far vision with IOL/ICL surgeries.

TABLE 3A
Exemplary Designs of EDOF Bifocal
Lenses in two aspheric sections

| | | EDOF | EDOF |
|---|---|---|---|
| | Parameters | Bifocal 1D | Bifocal3D |
| Centra | Radius R₁ (mm) | 1.15 | 1.10 |
| Aspherical | S.A. S₁ (Microns) | 0.70 | 1.0 |
| Zone | Focus offset Φ₁ | 1.0 | 1.65 |
| | (Diopter) | | |
| Annular | Radius R₂ (mm) | 1.75 | 1.75 |
| Aspherical | S.A. S₂ (Microns) | −1.11 | −2.22 |
| Zone | Focus offset Φ₂ | 0.37 | 1.15 |
| | (Diopter) | | |

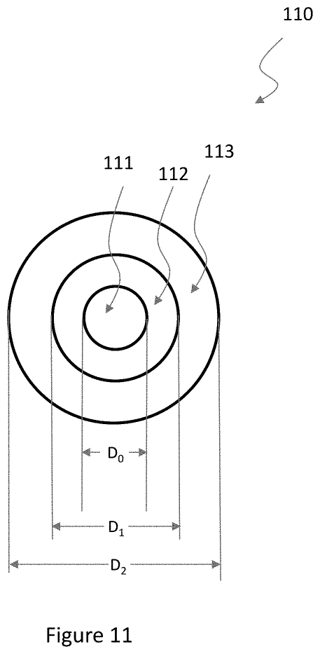

In a non-limiting embodiment, the EDOF bifocal lens in FIG. 11 for an eye (110) is configured as an implantable or a wearable lens, and comprises: 1) a baseline Diopter power extending across an optical section of the lens (111, 112, 113) for correction of far vision defects, and the optical section including the center section (111), the middle annular section (112), and outer annular section (113), and has a total diameter D2 between 5 mm and 8 mm, 2) a positive focus offset ϕ₁ less than 2.0 D and larger than +0.25 D at the center section (111) having a diameter less than 2.5 mm and larger than 1.8 mm, 3) two aspherical sections (111 and 112) having an outer diameter less than 4.5 mm and larger than 2.5 mm that covers at least the central pupil of an eye, and the aspherical section is characterized in that at least one surface of the lens is aspheric for inducing a positive spherical aberration in a first zone (111) and a negative spherical aberration in a second zone (112). The first and second zones are concentric. The second zone can further be configured to have a positive focus offset less than 1.5 D in some embodiments. The wavefront EDOF bifocal lens can be configured as a contact lens, an Intraocular Lenses (IOL), an Accommodating Intraocular Lenses (AIOL), an ICL(Implantable Contact Lens or Implantable Collamer Lens), or a Phakic IOL, which works with the cornea and crystalline lens of the eye together.

In the first exemplary design, we provide an EDOF bifocal with an add-on power of 1.0 D +/−0.25 D between two foci. Parameters of the exemplary wavefront bifocal lens (labeled “EDOF Bifocal1 D”) are listed in Table 3A.

We assume the EDOF bifocal lens has an optical section that has a diameter between 5 mm and 8 mm. The lens has a baseline Diopter power extending across an optical section of the lens for the correction of far vision defects the same as a monofocal lens.

The bifocal lens also has two aspherical sections that cover a central pupil of an eye, and its outer diameter D₀ is 3.5 mm (radius of 1.875). The aspherical sections are characterized in that at least one surface of the lens is aspheric for inducing a positive spherical aberration in a first zone and a negative spherical aberration in a second zone. The induced spherical aberrations in the aspherical sections are expressed as wavefront errors (OPD) across eye's pupil, i.e.,

$$\begin{matrix}OPD ( ρ ) = & 0.7 * {( ρ \text{/} {r}_{0} )}^{4} & if ρ \text{<=} 1.15 \\ = & - 1.11 * {( ρ \text{/} {r}_{1} )}^{4} & if 1.15 < ρ \text{<=} 1.75\end{matrix}$$

where ρ is a polar radius in the pupil plane. The positive spherical aberration in the first zone has its peak value of 0.70 microns at its boundary ρ=r₀=1.15. The negative spherical aberration in the second zone has its peak value of −1.11 microns at its boundary ρ=r₁=1.75 mm. The aspherical section has a diameter of 3.5 mm, covering a central pupil of the eye.

In addition to the baseline Diopter power and the induced spherical aberrations in aspherical sections, there is a positive focus offset of 1.0 D in the central (first) zone and a positive focus offset of 0.37 D in the annular (second) zone.

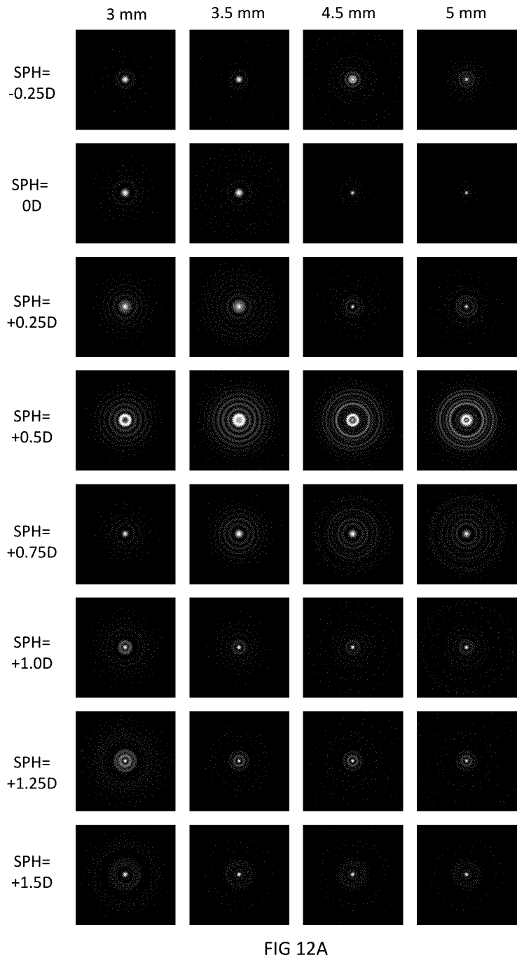

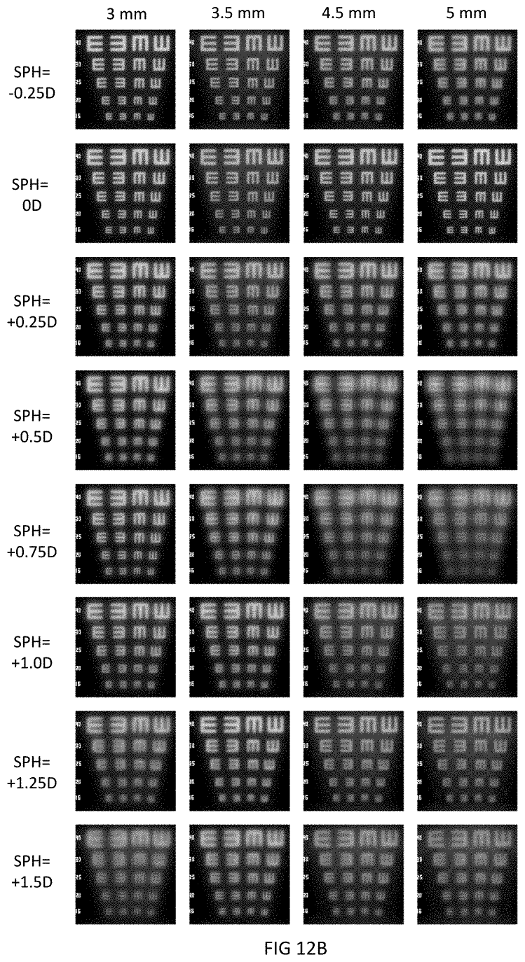

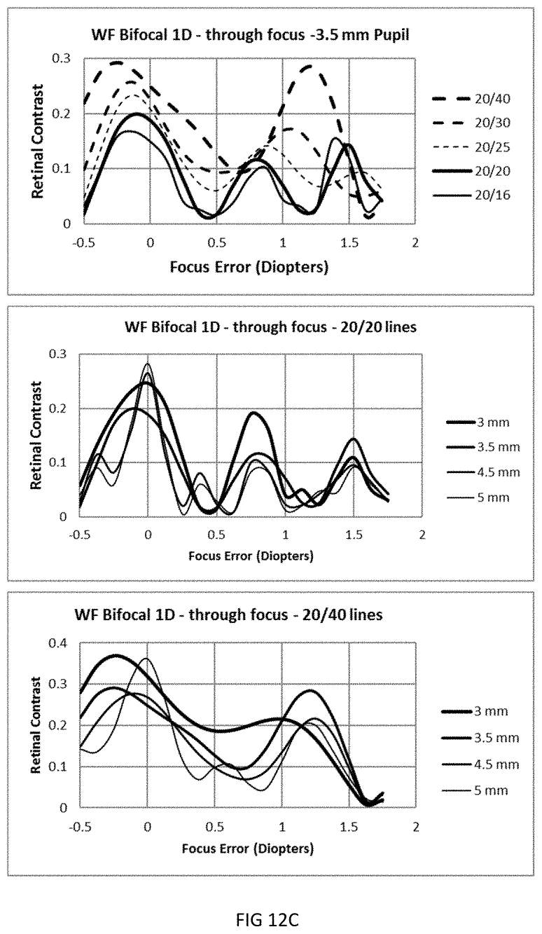

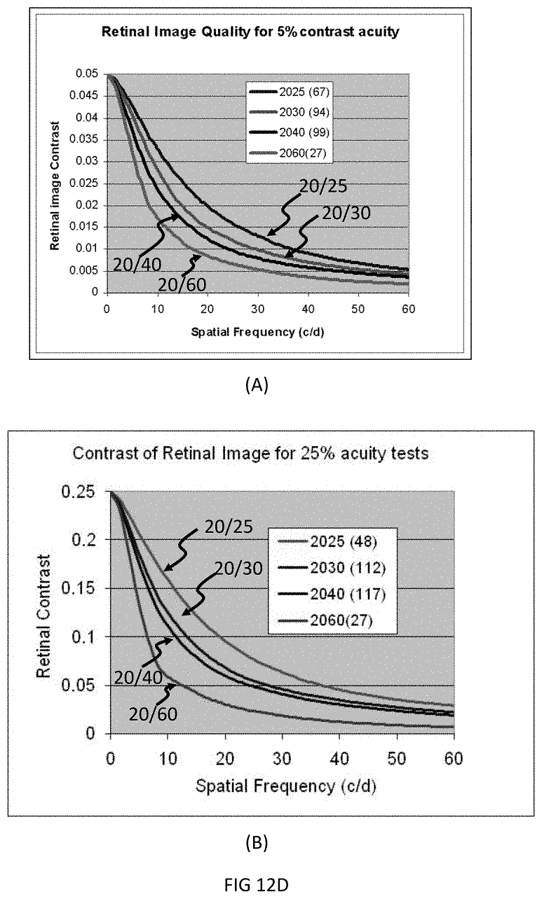

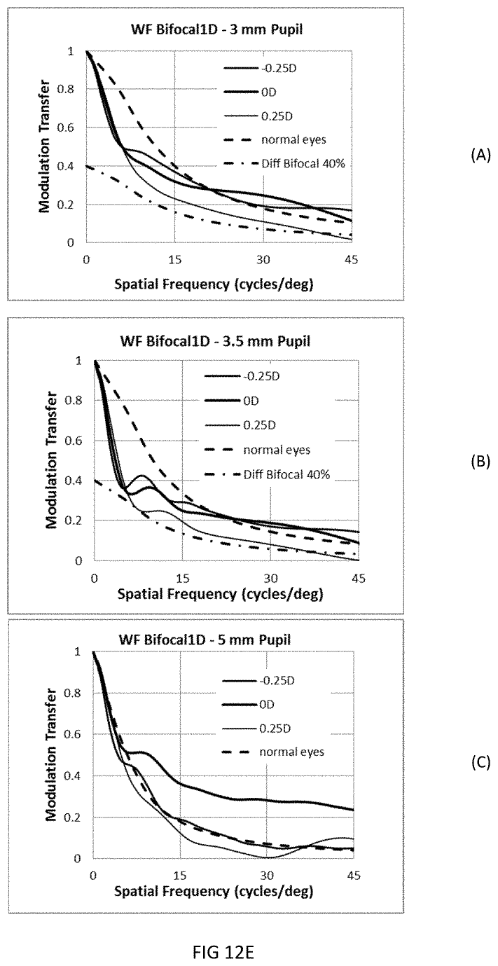

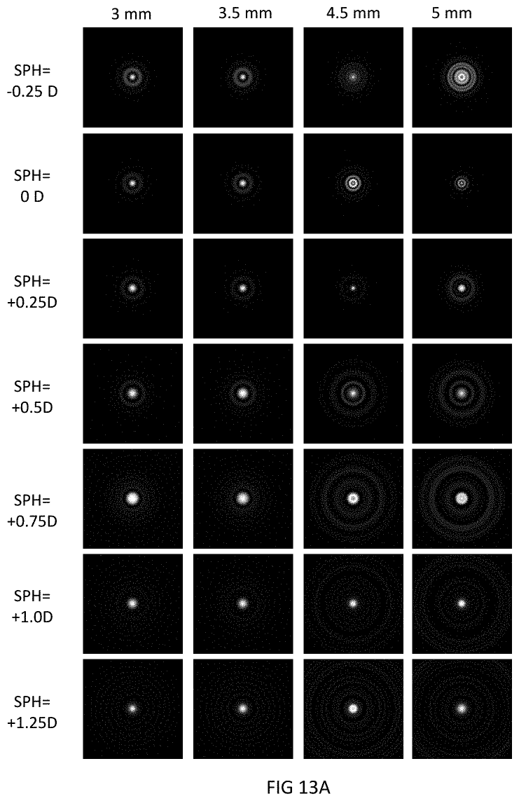

Performance of the wavefront bifocal lens is simulated and shown in FIG. 12A for calculated Points Spread Functions (PSF) from SPH=−0.25 D to SPH=+1.5 D and in FIG. 12B for calculated retinal images of an acuity chart. The parameter SPH is used to specify a focus error of the eye through focus. SPH=0 D specifies the best corrected vision at 4 meters, a typical distance for vision tests in the United States. SPH=−0.25 D specifies the corrected vision at infinity, which is myopic by −0.25 D if the targeted far distance is at 4 meters for the conventional acuity test. SPH=+1.0 D specifies a presbyopia correction of +1.0 D. We considered four pupil sizes of 3.0 mm and 3.5 mm for acuity tests, and 4.5 mm and 5.0 mm in diameter for night vision.

It is observed that, differing from the PSFs in FIG. 10A and FIG. 10C, the calculated PSFs of the WF Bifocal1D lens in FIG. 12A have a first focus covering focus range at least between −0.25 D and +0.25 D, and a second focus covering a focus range between +0.75 D and +1.5 D.

FIG. 12C shows plots of calculated “through focus” retinal contrast of WF Bifocal 1 D for a 3.5 mm pupil, and for 20/20 lines and 20/40 lines with pupil size between 3 mm to 5 mm. Our EDOF bifocal1D behaves slightly different from traditional bifocal in two aspects. First, the first focus for far distances is an Extended Depth of focus between −⅜ D and +⅜ D for acuity test at a 3.0 mm and 3.5 mm pupil. Second, the second focus for presbyopia correction between +0.75 D and +1.5 D has a gap for 20/20 acuity at +1.25 D. The calculated retinal images in FIG. 12B confirmed the wavefront bifocal characteristics plus a slightly degraded acuity and vision at +1.25 D

Estimating the best corrected acuity from through-focus MTF in FIG. 12C requires knowing the threshold contrast for each acuity line. FIG. 12D shows calculated retinal contrast for 20/25, 20/30, 20/40,20/60 for normal eyes in a photopic condition (A) and in Mesopic condition (B), respectively. These are unpublised data, and were obtained by J Liang, D Tanzer, T Brunstetter in studying more than 250 eyes of US navy pilots who had habitual and uncorrected acuity between 20/20 and 20/10. The photopic curves on the top (A) was obtained from 1) the best subjective acuity for each subject reading a chart of 5% low contrast acuity in a photopic condition, 2) the calculated MTF of each eye during the subject test of 5% low contrast acuity. From (A) in FIG. 12D, we estimate that the average threshold contrast for photopic vision is less than 2% for 20/25 (24 cycles/deg), for 20/30 (20 cycles/deg), and for 20/40 (15 cycles/deg). The Mesopic curves (B) was obtained from 1) the best subjective acuity for each eye reading a chart of 25% low contrast in a mesopic condition, 2) the calculated MTF of each eye for the pupil size during the subjective test of 25% low contrast acuity. From (B) in FIG. 12D, we estimate the average threshold contrast for mesopic vision is about 5% to 6% for 20/25 (24 cycles/deg), for 20/30 (20 cycles/deg), and for 20/40 (15 cycles/deg).

FIG. 12E shows plots of calculated Modulation Transfer Function (MTF) of WF Bifocal 1 D for far distances at infinity (−0.25 D), at 4 meters (0 D), and a focus error at +0.25 D for pupil sizes of 3 mm, 3.5 mm, and 5 mm. In FIG. 12E, we also show the mean MTFs of normal eyes labed as “Normal Eyes”, which is calculated based on the formula provided by A B Watson in Journal of Vision, 13 (6):18, pp. 1-11 (2013), as well as estimated MTFs of a diffractive bifocal lenses labled as “Diff Bifocal 40%” that is calculated from the mean MTF from normal eyes with a bifocal of equally 50%. Diffractive bifocal lenses usualy have an energy loss of about 20% that does not contribute to neither of “0” or “1” order diffraction image. Our WF Bifocal 1 D offers better contrast than diffractive multifocal lenses with 50% at far distances, and will have no contrast loss for spatial frequencies larger than 20 c/deg (20/30 or finer features) and a slight contrast loss for spatial frequencies less than 20 c/deg, when compared to normal human eyes. This is particularly true for real eyes because uncorrected astigmatism and coma in an eye can be mitigated by our WF Bifocal 1 D lenses, and they will degrade quality of vision for conventional monofocal lenses and diffractive multifocal lenses.

From data in FIG. 12C and FIG. 12E, we have a few findings for the EDOF bifocal1D. First, we expect the EDOF Bifocal can offer the patient 20/16 or better acuity with relatively high contrast. Second, night vision for a pupil size of 4.5 mm and 5 mm will be exceptional for far distances. Therefore, a bifocal lens for a presbyopia correction of 1 D is invented with little or no loss in retinal contrast at far distances. Another advantage of the wavefront bifocal lenses is its tolerance to uncorrected astigmatism (about 0.5 D).

In the exemplary design of “EDOF bifocal3 D” in Table 3A, the bifocal lens also has aspherical sections covering a central pupil of an eye. The induced spherical aberrations in the aspherical sections are expressed as wavefront errors (OPD) across eye's pupil, or

$$\begin{matrix}OPD ( ρ ) = & 1.0 * {( ρ \text{/} {r}_{0} )}^{4} & if ρ < {r}_{0} = 1.1 \\ = & - 2.22 * {( ρ \text{/} {r}_{1} )}^{4} & if 1.1 < ρ \text{<=} {r}_{1} = 1.75\end{matrix}$$

where ρ is a polar radius in the pupil plane. The positive spherical aberration in the first zone has its peak value of 1.0 microns at its boundary ρ=r₀=1.1. The negative spherical aberration in the second zone has its peak value of −2.22 microns at its boundary ρ=r₁=1.75.

In addition to the baseline Diopter power and the induced spherical aberrations in the aspherical sections, there is a positive focus offset of 1.65 D in the central (first) zone and a positive focus offset of 1.15 D in the annular (second) zone.

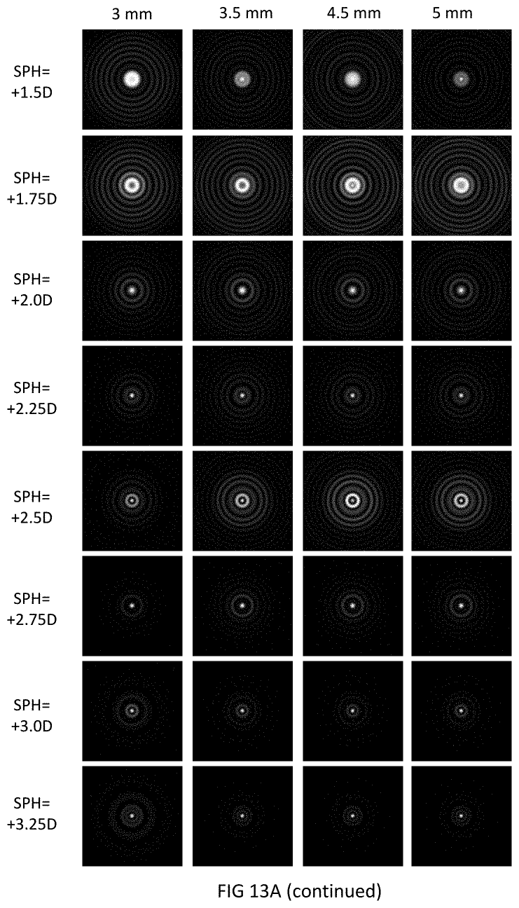

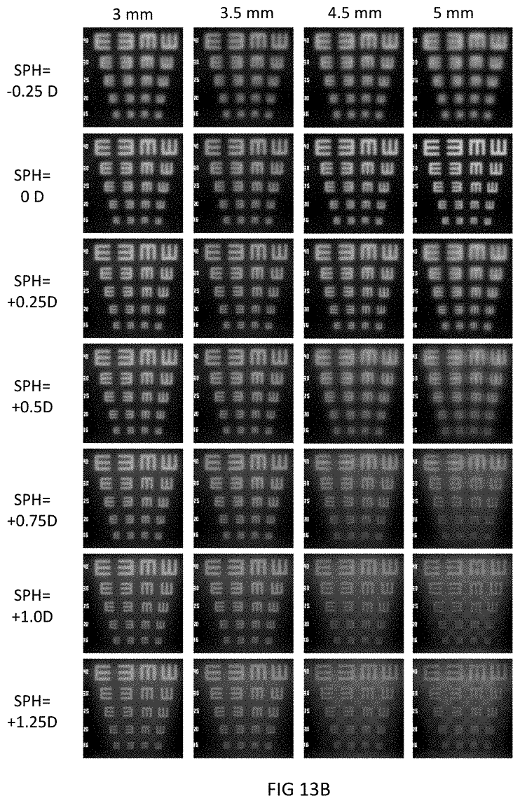

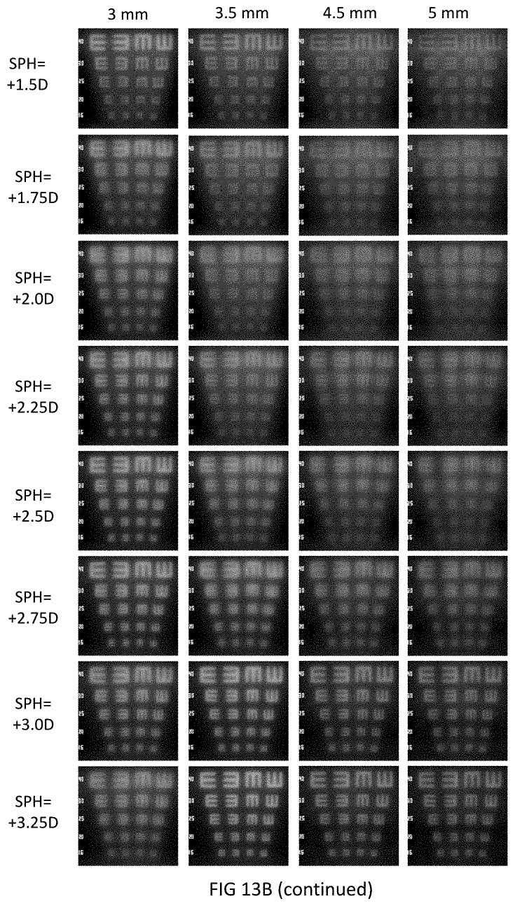

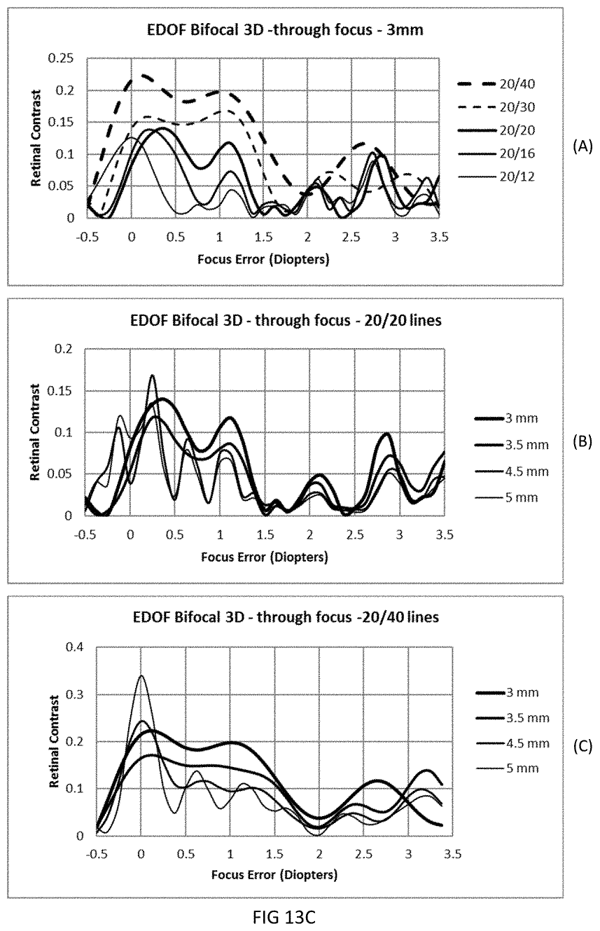

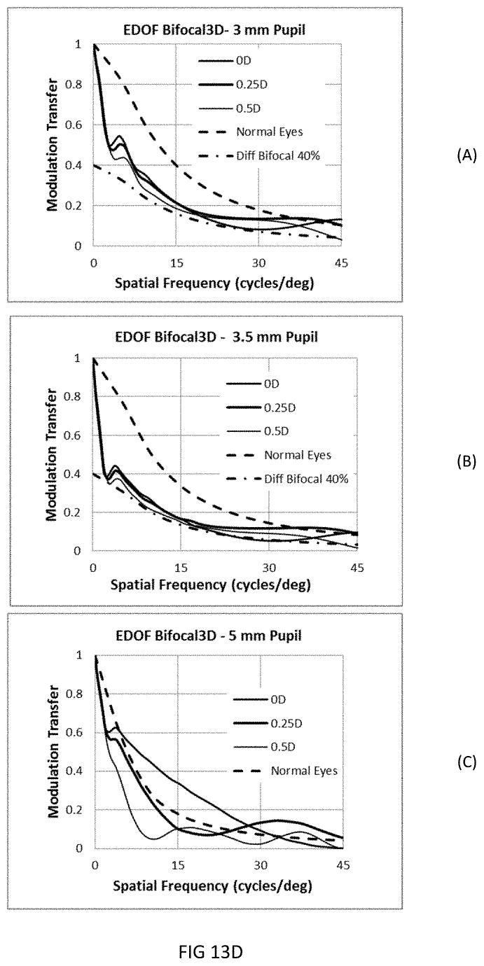

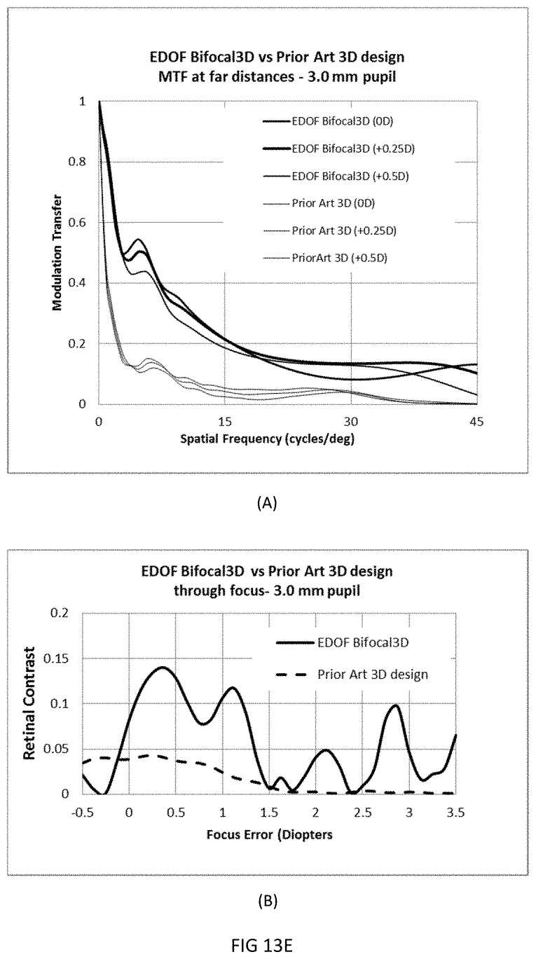

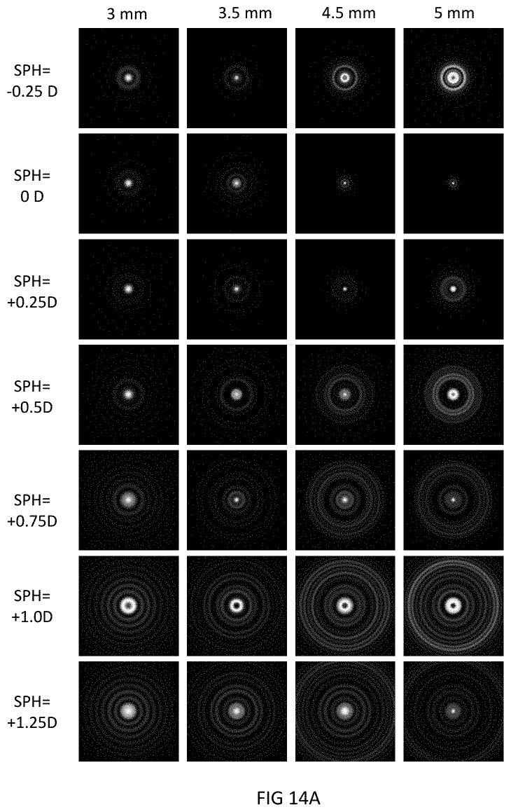

Performance of the wavefront EDOF bifocal3 D is simulated and shown in FIG. 13A for calculated Point Spread Functions (PSF) from SPH=−0.25 D to SPH=+3.25 D and in FIG. 13B for calculated retinal images of an acuity chart. SPH=0D specifies the best corrected vision at 4 meters, a typical distance for vision tests in the United States. SPH=−0.25 D specifies the corrected vision at infinity, SPH=+3.0 D specifies a presbyopia correction of +3.0 D. We considered four pupil sizes of 3.0 mm and 3.5 mm for acuity tests, and 4.5 mm and 5.0 mm for night vision.

It is observed that the calculated PSFs of the WF Bifocal3 D lens in FIG. 13A have a first focus covering an extended focus range between 0 D and +1.25 D, and a second focus covering a focus range between +2.75 D and 3.25 D. A focus at +2.25 D is too narrow and too weak to be considered a focus region.

FIG. 13C shows plots of calculated “through focus” retinal contrast of EDOF Bifocal 3 D for a 3.5 mm pupil, and for 20/20 lines and 20/40 lines with pupil size between 3 mm to 5 mm. Our EDOF bifocal3 D behaves slightly different from traditional bifocal in two aspects. First, the first focus for far distances is an Extended Depth of focus between 0 D and +1.25 D for acuity test at a 3.0 mm and 3.5 mm pupil. Second, a second focus for presbyopia correction between +2.75 D and +3.25 D. The calculated retinal images in FIG. 13B confirmed the EDOF bifocal characteristics.

FIG. 13D shows plots of calculated Modulation Transfer Function (MTF) of WF Bifocal 3 D for far distances at infinity (−0.25 D), at 4 meters (0 D), and a focus error at +0.25 D for pupil sizes of 3 mm, 3.5 mm, and 5 mm. In FIG. 13D, we also show the mean MTFs of normal eyes labed as “Normal Eyes” as well as estimated MTFs of a diffractive bifocal lenses labled as “Diff Bifocal 40%”. Our WF Bifocal 3 D will offer equal or better contrast than diffractive multifocal lenses at far distances, and will have no contrast loss for spatial freqencies larger than 30 c/deg (20/20 or finer features) and a slight contrast loss for for spatial freqencies lenss than 30 c/deg, when compared to normal human eyes. This is particularly true for real eyes because uncorrected astigmatism and coma in an eye can be mitigated by our WF Bifocal 3 D lenses, and they will degrade quality of vision for conventional monofocal lenses and diffractive multifocal lenses.

From FIG. 13C and FIG. 13D, we have a few findings for the EDOF bifocal3 D lens. First, we expect the EDOF Bifocal can offer the patient 20/16 or better acuity with high contrast and an extended depth of focus. Second, night vision for a pupil size of 4.5 mm and 5 mm will be excellent for far distances as well as for near distances. Another advantage of the wavefront bifocal lenses is its tolerance to uncorrected astigmatism of up to 0.5 D.

Solving the problem of poor contrast at far distances with the wavefront design in the prior art (U.S. Pat. No. 8,529,559 B2 and US patent application # 2011/0029073 A1) is made possible by finding an optimized solution with a reduced focus offset of +1.65 D in the central aspheric section with the EDOF Bifocal3 D, being 1.35 D less than a total focus depth of 3 D for the wavefront bifocal lenses. On the contrary, a focus offset of +4.0 D in the central aspheric section was found for the wavefront design in the prior art, being 1.0 D larger than a total focus depth of 3 D. Significant improvement in contrast by our EDOF Bifocal3 D in the present invention is plotted in FIG. 13E, showing retinal contrast for far distances in (A) and through-focus contrast for 20/20 acuity in (B) of our new EDOF Bifocal 3 D in comparison to the wavefront design in the prior art (U.S. Pat. No. 8,529,559 B2 and US patent application # 2011/0029073 Al). FIG. 13E are obtained for a lens diameter of 3 mm, a dimension for testing multifocal lenses in industry standards.

In one embodiment, the induced spherical aberrations in the aspherical sections is expressed as wavefront errors across the pupil or OPD, or

$$\begin{matrix}OPD ( ρ ) = & {S}_{1} * {( ρ \text{/} {r}_{0} )}^{4} & if ρ \text{<=} {r}_{0} \\ = & ( - {S}_{2} ) * {( ρ \text{/} {r}_{1} )}^{4} & if {r}_{0} < ρ \text{<=} {r}_{1}\end{matrix}$$

where ρ is a polar radius in a pupil plane, S₁ is positive and it measures the positive spherical aberration in the first zone (111), and r₀=0.5*D₀ is the radius of the first zone larger than 0.87 mm and less than 1.25 mm. (−S₂) is negative and it measures the negative spherical aberration in the second zone, and r₁ is the outer diameter of the second zone (112) less than 2.25 mm and larger than 1.20 mm. The second zone of the aspherical section can be further configured to add a focus offset ϕ₂, wherein the focus offset is between −1.0 D and +1.0 D. The positive spherical aberration S₁ in one embodiment is larger than 0.20 microns and less than 1.50 microns. Table 3B lists the calculated positive spherical aberration for the wavefront bifocal lenses with a diameter of the central aspherical section between 1.75 mm and 2.4 mm. The negative spherical aberration (−S₂) in one embodiment is more than 0.25 and less than 6 microns in magnitude. Table 3C lists the calculated negative spherical aberration for the wavefront bifocal lenses with an outer diameter of the annular aspherical section between 2.5 mm and 4.4 mm.

In still another embodiment, the aspherical section further induces a generalized spherical aberration that is characterized as the summation of a plurality of terms of ρⁿ, wherein n is an integer equal to or greater than 3.

In some embodiments, the wavefront bifocal lens is configured as a bifocal contact lens having a diameter between 9 mm and 16 mm. The wavefront bifocal contact lens has a front surface and a back surface, and at least one of the front surface and the back surface is aspherical at the lens center.

In one embodiment, the back surface of the wavefront EDOF bifocal contact lens is further configured to have an aspheric shape at a lens periphery for preventing lens rotation on the eye if the lens is a toric bifocal contact lens.

TABLE 3B
Positive spherical aberration in the central zone of the wavefront bifocals

| | Diameter of the central | | | | |
|---|---|---|---|---|---|
| | aspherical section | D₁ (mm) | 1.75 | 2.1 | 2.4 |
| EDOF | Spherical aberration in the | S₁ (μm) | 0.23 | 0.48 | 0.83 |
| Bifocal 1D | central section = 0.70* (D₁/2.3)⁴ | | | | |
| EDOF | Spherical aberration in the | S₁ (μm) | 0.40 | 0.83 | 1.42 |
| Bifocal3D | central section = 1.0* (D₁/2.2)⁴ | | | | |

TABLE 3C
Negative spherical aberration in the annular section of the wavefront bifocals

| | Outer diameter of the annular | | | | |
|---|---|---|---|---|---|
| | aspherical section | D₂ (mm) | 2.5 | 3.5 | 4.4 |
| EDOF | Spherical aberration in the | S₂ (μm) | −0.29 | −1.11 | −2.78 |
| Bifocal 1D | annular section = −1.11* (D₂/3.5)⁴ | | | | |
| EDOF | Spherical aberration in the | S₂ (μm) | −0.57 | −2.22 | −5.55 |
| Bifocal3D | annular section = −2.22* (D₂/3.5)⁴ | | | | |

In some embodiments, the wavefront bifocal lens is configured as a wavefront bifocal IOL that has a diameter between 5 mm and 7 mm, and the aspheric surface is a front surface or a back surface of the IOL. In one embodiment, the wavefront bifocal IOL is further configured as an accommodating IOL.

In another embodiment, the wavefront bifocal lens is configured as a wavefront cornea inlay that has a diameter of about 6 mm or between 5 mm and 7 mm, and the aspheric surface is a front surface or a back surface of the corneal inlay.

### 3. Wavefront EDOF Trifocal Lenses

Diffractive trifocal IOLs not only provide a high rate for spectacle-free IOL surgeries, but also make post-op eyes see things that actually do not exist and are created by the diffractive optics: 1) nighttime symptoms of halo and starburst due to simultaneous multiple images, 2) spider-web type of night symptoms associated with diffractive structures, 3) ghost images of large objects at distance caused by defocused intermediate and near foci.

Inducing spherical aberration of opposite sign in the central pupil was proposed in U.S. Pat. No. 8,529,559 B2 and US patent application # 2011/0029073 A1 for presbyopia-correcting IOLs of +3 D. In order to obtain a desired 3 D Depth of Focus (DoF), a focus offset of +4.0 D larger than the desired DOF was introduced in the central aspheric section.

TABLE 4A
Exemplary designs of wavefront trifocal lenses in aspheric zones

| | | EDOF | EDOF | EDOF | EDOF |
|---|---|---|---|---|---|
| | | Trifocal | Trifocal | Trifocal | Trifocal |
| | Parameters | 2.25Da | 2.25Db | 2.75D | 3.25D |
| Central | Radius R₁ (mm) | 1.0 | 0.95 | 0.92 | 0.875 |
| Aspherical | S.A. S₁ (Microns) | 0.75 | 0.75 | 0.8 | 0.90 |
| Zone | Focus offset Φ₁ | 1.62 | 1.75 | 2.0 | 2.70 |
| | (Diopter) | | | | |
| Annular | Radius R₂ (mm) | 1.5 | 1.5 | 1.5 | 1.5 |
| Aspherical | S.A. S₂ (Microns) | −1.2 | −2.20 | −2.20 | −3.4 |
| Zone | Focus offset Φ₂ | 0.5 | −0.5 | 0.12 | 0 |
| | Diopter) | | | | |

There are at least three issues with the design in U.S. Pat. No. 8,529,559 B2 and US patent application # 2011/0029073 A1. First, the design suffers from a low contrast at far distances, which was noticed and addressed with an improved design of Mini Well Ready IOLs. Second, the original design as well as Mini Well Ready IOL are not trifocal lenses for meeting active lifestyle of patients that require excellent vision for far distances for driving and watching TV, intermediate distances (around 0.6 m) for working with computers, and near distances (around 0.3 m) for reading books or small prints. Third, there also lack trifocal ophthalmic lenses with a total focus range between 2.0 D and 2.5 D for contact lenses, implantable contact lenses, and corneal inlays, since these lenses work together with eye's crystalline lens.

In one aspect of the present invention, we provide a new class of wavefront EDOF trifocal lenses in Table 4A to address these issues. First, we were able to create wavefront trifocal lenses that have three foci: a first “far” focus, a second “intermediate” focus with a small add-on power, and a third “near” focus with a large add-on power. These trifocal lenses offer functional vision for “far” distances, “intermediate” distances, and “near” distances. Second, the trifocal lenses cover a broad presbyopia range from 2.25 D to 3.25 D not only for IOLs but also for contact lenses, ICLs and cornea inlays. Third, solving the problem of poor contrast with far distances for a presbyopia correction of 3 D, which was made possible by discovering optimized solutions that use a focus offset ϕ₁ smaller than a total presbyopia range from the baseline Diopter power to the “near” add-on power. Fourth, the trifocal lenses have an extended depth of focus for far distances.

In one exemplary design of “EDOF Trifocal 2.75 D” in Table 4A, the lens has two aspherical sections covering a central pupil of an eye, and its outer diameter is 3.0 mm. The aspherical sections are characterized in that at least one surface of the lens is aspheric for inducing a positive spherical aberration in a first zone and a negative spherical aberration in a second zone, and the first and second zones are concentric. The induced spherical aberrations in the aspherical sections are expressed as wavefront errors (OPD) across eye's pupil, or

$$\begin{matrix}OPD ( ρ ) = & 0.80 * {( ρ \text{/} {r}_{0} )}^{4} & if ρ < {r}_{0} = 0.92 \\ = & - 2.2 * {( ρ \text{/} {r}_{1} )}^{4} & if 0.92 < ρ \text{<=} {r}_{1} = 1.5\end{matrix}$$

where ρ is a polar radius in the pupil plane. The positive spherical aberration in the first zone has its peak value of 0.80 microns at its boundary ρ=r₀=0.92. The negative spherical aberration in the second zone has its peak value of −2.2 microns at its boundary ρ=r₁=1.5.

In addition to the baseline Diopter power and the induced spherical aberrations in the aspherical sections, there is a positive focus offset of +2.0 D in the central (first) zone with a diameter of 1.75 mm (radius of 0.875 mm).

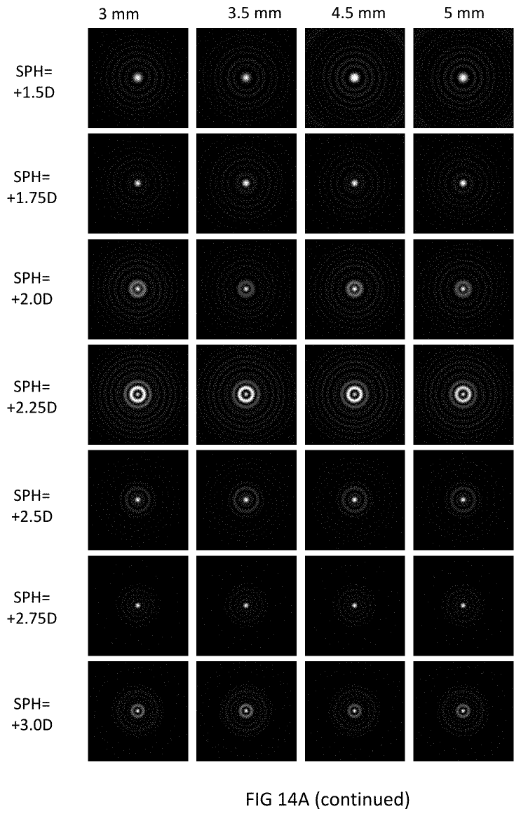

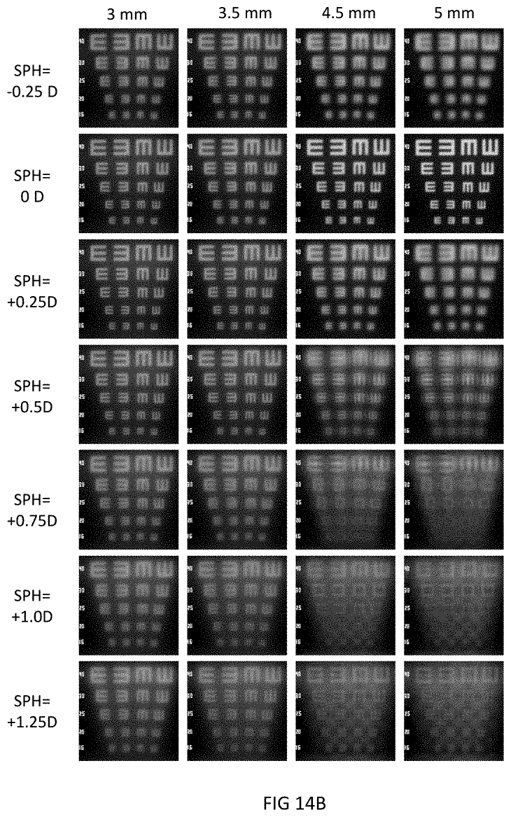

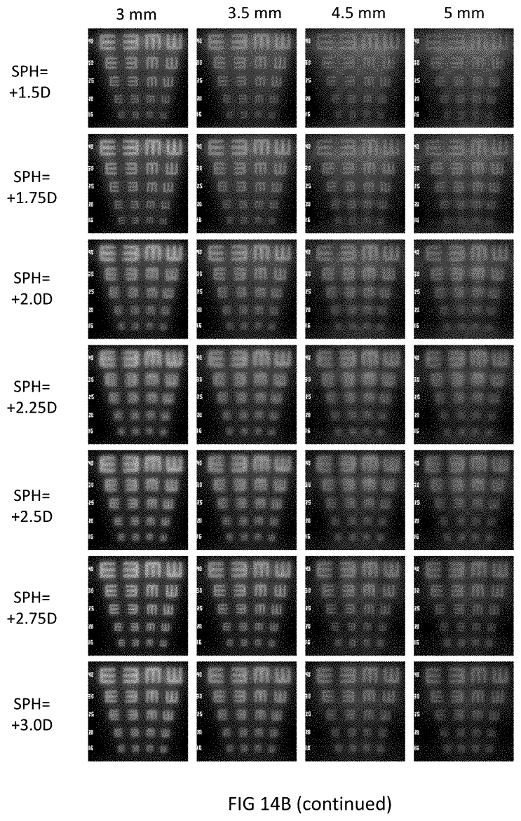

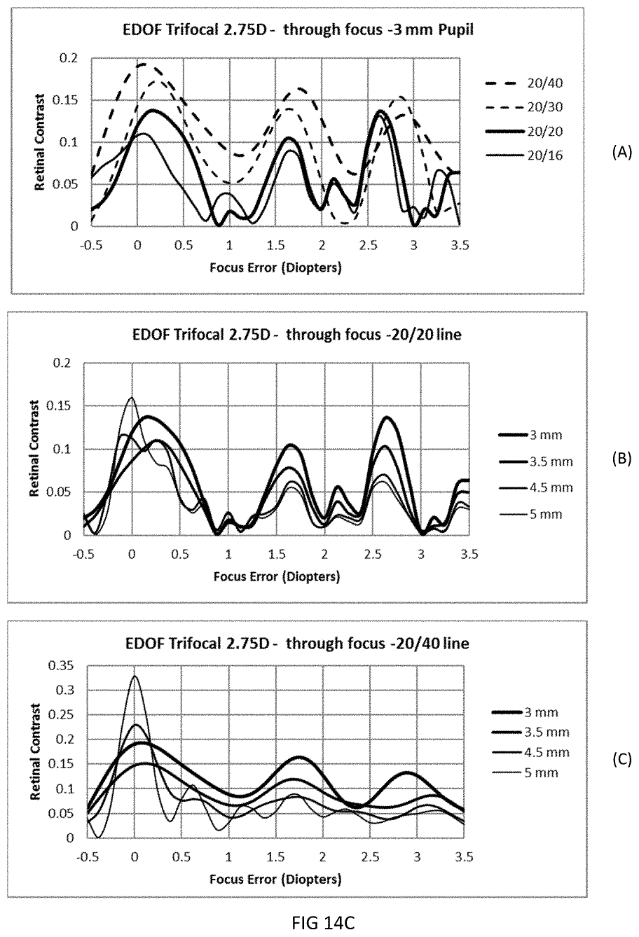

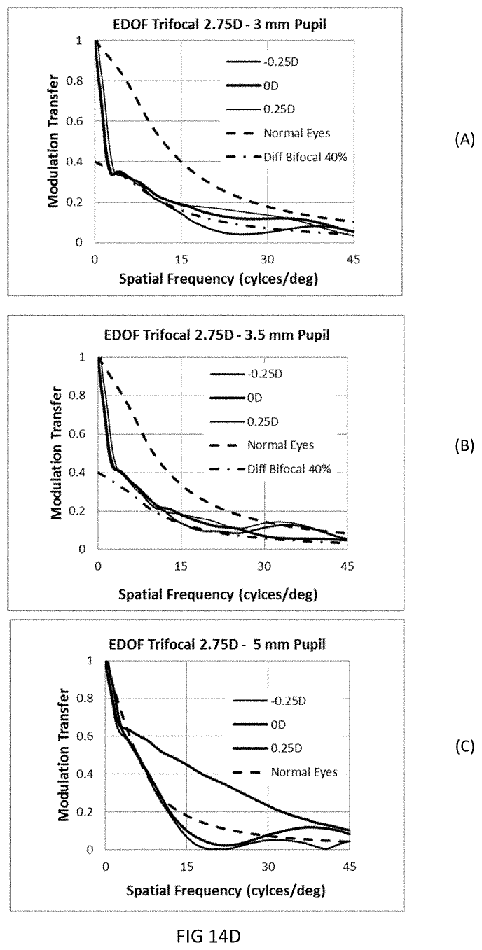

Performance of the EDOF trifocal 2.75 D is simulated and shown in FIG. 14A for the calculated Point Spread Functions (PSF) from −0.25 D to +3.0 D and in FIG. 14B for the calculated retinal images of an acuity chart. The parameter SPH is used to specify a focus error of the eye through focus. SPH=0 D specifies the best corrected vision at 4 meters. SPH=−0.25 D specifies the corrected vision at infinity, which is myopic by −0.25 D if the targeted far distance is at 4 meters for the conventional acuity test. SPH=+3.0 D specifies a presbyopia correction of +3.0 D. We considered four different pupil sizes of 3.0 mm and 3.5 mm for acuity tests, and 4.5 mm and 5.0 mm in diameter for night vision.

FIG. 14C shows plots of calculated “through focus” retinal contrast of EDOF trifocal 2.75 D for a 3.5 mm pupil, and for 20/20 lines and 20/40 lines.

It is observed from the calculated PSFs in FIG. 14A and the “through focus” plots in FIG. 14C that the EDOF trifocal 2.75 D has three distinct foci: a first focus covering an extended focus range between −0.25 D and +0.75 D for vision at far distance, a second focus covering a focus range between +1.25 D and +2.0 D for intermidiate distances, and a third focus between 2.25 D and 3.0 D for near distance.

FIG. 14D shows plots of calculated Modulation Transfer Function (MTF) of EDOF trifocal 2.75 D for far distances at infinity (−0.25 D), at 4 meters (0 D), and a focus error at +0.25 D for pupil sizes of 3 mm, 3.5 mm, and 5 mm. In FIG. 14D, we also show the mean MTFs of normal eyes labed as “Normal Eyes” as well as estimated MTFs of a diffractive bifocal lenses labled as “Diff Bifocal 40%”. Our EDOF trifocal 2.75 D will offer equal or better contrast than diffractive multifocal lenses at far distances, and will have no contrast loss for spatial frequencies larger than 30 c/deg (20/20 or finer features) and some contrast loss for spatial frequencies less than 30 c/deg, when compared to normal eyes. This is particularly true for real eyes because uncorrected astigmatism and coma in an eye can be mitigated by our EDOF trifocal 2.75 D lenses, and they will degrade quality of vision for conventional monofocal lenses and diffractive multifocal lenses.

From FIG. 14C and FIG. 14D, we have a few findings for the EDOF trifocal 2.75 D lens. First, we expect the EDOF Bifocal can offer 20/16 or better acuity with relatively high contrast and an extended depth of focus. Second, night vision for a pupil size of 4.5 mm and 5 mm will be excellent for far distances as well as for near distances. Another advantage of the wavefront bifocal lenses is its tolerance to uncorrected astigmatism up to 0.5 D.

Table 4A provides three other embodiments of EDOF trifocal lenses that solve the problem of low contrast for far distance with the designs in U.S. Pat. No. 8,529,559 B2 and US patent application # 2011/0029073 A1 PLUS the following features: 1) an extended depth of focus for far distances, 2) a second focus with presbyopia correction between +1.25 D and +1.75 D, 3) a third focus that extends the total focus range between 2.25 D and 3.25 D.

TABLE 4B
Positive soherical aberration in the central zone of EDOF trifocal lenses

| | Diameter of central section | D₁ (mm) | 1.65 | 1.85 | 2.1 |
|---|---|---|---|---|---|
| EDOF | Spherical aberration in central | S₁ (μm) | 0.35 | 0.55 | 0.91 |
| Trifocal2.25Da | section = 0.75* (D₁/2.0)⁴ | | | | |
| EDOF | Spherical aberration in central | S₁ (μm) | 0.43 | 0.67 | 1.12 |
| Trifocal2.25Db | section = 0.75* (D₁/1.9)⁴ | | | | |
| EDOF | Spherical aberration in central | S₁ (μm) | 0.52 | 0.82 | 1.36 |
| Trifocal2.75D | section = 0.80* (D₁/1.84)⁴ | | | | |
| EDOF | Spherical aberration in central | S₁ (μm) | 0.71 | 1.12 | 1.86 |
| Trifocal3.25D | section = 0.90* (D₁/1.75)⁴ | | | | |

TABLE 4C
Negative spherical aberration in the annular aspherical zone of trifocal lenses

| | Outer diameter of annular | | | | |
|---|---|---|---|---|---|
| | aspherical section | D₂ (mm) | 2.5 | 3.0 | 3.75 |
| EDOF | Spherical aberration in the | S₁ (μm) | −0.57 | −1.20 | −2.92 |
| Trifocal2.25Da | annular section = −1.2* (D₂/3.O)⁴ | | | | |
| EDOF | Spherical aberration in the | S₁ (μm) | −1.06 | −2.20 | −5.37 |
| Trifocal2.25Db | annular section = −2.2* (D₂/3.O)⁴ | | | | |
| EDOF | Spherical aberration in the | S₁ (μm) | −1.06 | −2.20 | −5.37 |
| Trifocal2.75D | center section = −2.2* (D₂/3.O)⁴ | | | | |
| EDOF | Spherical aberration in the | S₁ (μm) | −1.64 | −3.40 | −8.3 |
| Trifocal3.25D | center section = −3.4* (D₂/3.O)⁴ | | | | |

The inventions of wavefront trifocal lenses with high retinal contrast at far distances are made possible by finding optimized solutions with a low focus offset of +1.62 D and +2.7 D in the central aspheric section. These EDOF trifocal designs can be adapted for contact lenses, IOLs, accommodating IOLs, phakic IOLs, ICLs, and corneal inlays.

In some embodiments, the wavefront EDOF trifocal lens in FIG. 11 is configured as an implantable or wearable lens. It comprises: 1) a baseline Diopter power extending across an optical section of the lens (111, 112, 113) for correction of far vision defects, and the optical section has a diameter D₂ between 5 mm and 8 mm and the correction of far vision defects including a focus error and/or a cylinder error, 2) a positive focus offset ϕ1 less than +3.0 D and larger than +1.0 D at a center section (111) having a diameter D0 less than 2.1 mm and larger than 1.65 mm, 3) two central aspherical sections (111, 112) at least in a center of the lens having an outer diameter less than 4 mm and larger than 2.5 mm, which covers a central pupil of the eye, and the central aspherical sections being characterized in that at least one surface of the lens is aspheric for inducing a positive spherical aberration in a first zone (111) and a negative spherical aberration in a second zone (112), and the first zone and the second zone are concentric. The wavefront errors beyond the baseline Diopter power convert the monofocal lens into a trifocal lens: a first “far” focus, a second focus with an “intermediate” add-on power, and a third focus with a “near” add-on power, wherein the positive focus offset ϕ₁ at a center section must be less than the total focus range of the trifocal lens.

In one embodiment of the wavefront EDOF trifocal lenses, the induced spherical aberrations in the aspherical sections are expressed in Optical Path Difference (OPD), or the wavefront errors across eye's pupil as

$$\begin{matrix}OPD ( ρ ) = & {S}_{1} * {( ρ \text{/} {r}_{0} )}^{4} & if ρ \text{<=} {r}_{0} \\ = & ( - {S}_{2} ) * {( ρ \text{/} {r}_{1} )}^{4} & if {r}_{0} < ρ \text{<=} {r}_{1}\end{matrix}$$

where ρ is a polar radius in the pupil plane. S₁ is positive and it measures the positive spherical aberration in the first zone having its peak value of S₁ at its boundary ρ=r₀, and r₀ is a radius of the first zone and is larger than 0.82 mm and less than 1.1 mm. (−S₂) is negative and it measures the negative spherical aberration in the second zone having its peak value of (−S₂) at its boundary ρ=r₁, and r₁ is the outer diameter of the second zone, which is larger than 1.2 mm and less than 2 mm.

In another embodiment, the positive spherical aberration in the first zone S1 is larger than 0.30 microns and less than 2 microns.

In yet another embodiment, the negative spherical aberration (−S2) is larger than 0.50 and less than 8.5 microns in magnitude.

In still another embodiment, the aspherical section further induces a generalized spherical aberration that is characterized as Optical Path Difference including terms of ρⁿ and n is an integer equal to or greater than 3.

In yet another embodiment, the wavefront trifocal lens is further configured to add a focus error ϕ₂ into the second zone of the aspheric section, and the focus error is between −1.0 D and +1.0 D.

In some embodiments, the wavefront trifocal lens is configured as a wavefront trifocal contact lens having a diameter between 9 mm and 16 mm, and the aspheric surface is either a front surface or a back surface of the contact lens. The back surface of the trifocal contact lens is further configured to have an aspheric shape at a lens periphery for preventing lens rotation on the eye if the contact lens is also a toric lens.

In other embodiments, the wavefront trifocal lens is configured as a wavefront trifocal IOL, and it has an optical section of about 6 mm, between 5 mm and 7 mm in diameter. The wavefront trifocal IOL has a front surface and a back surface, and at least one of the front or back surface is aspheric at the lens center.

### 4. Quasi-Accommodating Lenses

Accommodating IOLs surfer from one or more of the following drawbacks today: 1) a low accommodation range being not enough for an effective presbyopia correction, 2) poor control of artificial accommodation to achieve a desired accommodation state at will, 3) a large fluctuation in artificial accommodation making vision unstable, 4) poor vision due to eye's uncorrected astigmatism.

In one aspect of the present invention, we disclose a new class of wavefront lenses for an eye: Quasi Accommodating and Continuously-In-Focus (QACIF) Lens. The QACIF lens has an optical section less than 8 mm in diameter and provides nearly continuous focus for a focus range more than 1.0 D and up to 2 D. Although the focusing range of 2 D is smaller than 3 D for IOLs used in cataract surgeries, a QACIF lens with a 2 D depth of focus will be good enough for treatments of all presbyopia eyes without cataract using an ICL, a phakic IOL, or a contact lens. QACIF lens can be achieved by a special multifocal structure that has a plurality of foci being close enough for creating a nearly continuous focus. The multifocal lenses can be achieved by 1) using an aspherical surface to induce spherical aberrations into the central part of lens with a diameter less than 4 mm, or 2) using diffractive optics to create simultaneous multiple foci.

In one exemplary design of a QACIF lens “QACIF2D” in Table 5A, the lens has two aspherical sections covering a central pupil of an eye, and its outer diameter is 3.5 mm. The aspherical sections are characterized in that at least one surface of the lens is aspheric for inducing a positive spherical aberration in a first zone and a negative spherical aberration in a second zone, and the first and second zones are concentric. The induced spherical aberrations in the aspherical sections are expressed as wavefront errors (OPD) across eye's pupil

$$\begin{matrix}OPD ( ρ ) = & 1.0 * {( ρ \text{/} {r}_{0} )}^{4} & if ρ < {r}_{0} = 1.25 \\ = & - 1.11 * {( ρ \text{/} {r}_{1} )}^{4} & if 1.25 < ρ \text{<=} {r}_{1} = 1.75\end{matrix}$$

where ρ is a polar radius in the pupil plane. The positive spherical aberration in the first zone has its peak value of 1.0 microns at its boundary ρ=r₀=1.25 mm. The negative spherical aberration in the second zone has its peak value of −1.11 microns at its boundary ρ=r₁=1.75 mm.

In addition to the baseline Diopter power and the induced spherical aberrations in the two aspherical sections, there is a positive focus offset of +1.25 D in the central (first) zone with a diameter of 2.5 mm (radius of 1.25 mm), and a positive focus offset of +0.75 D in the annular (second) zone with an outer diameter of 3.5 mm (radius of 1.75 mm).

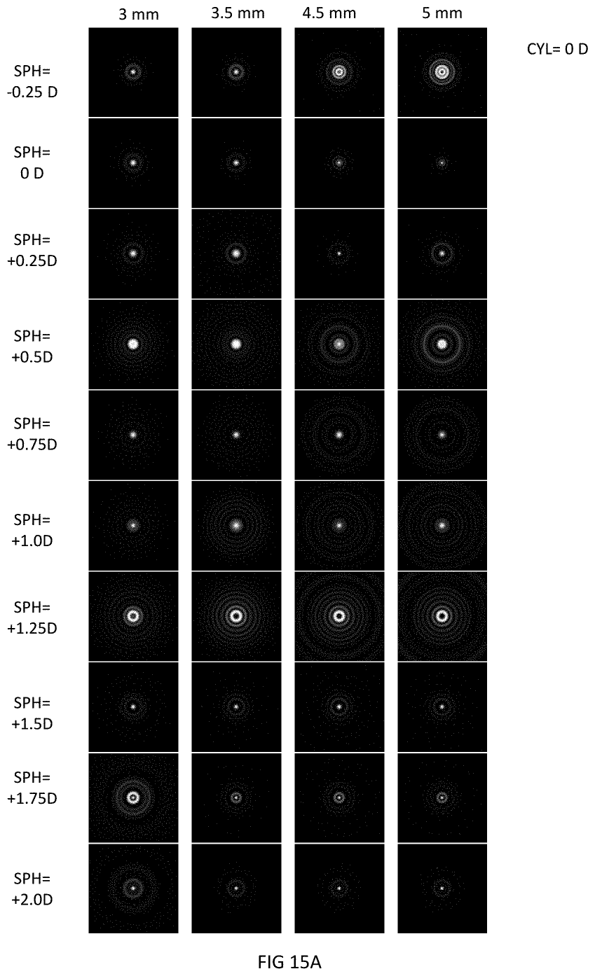

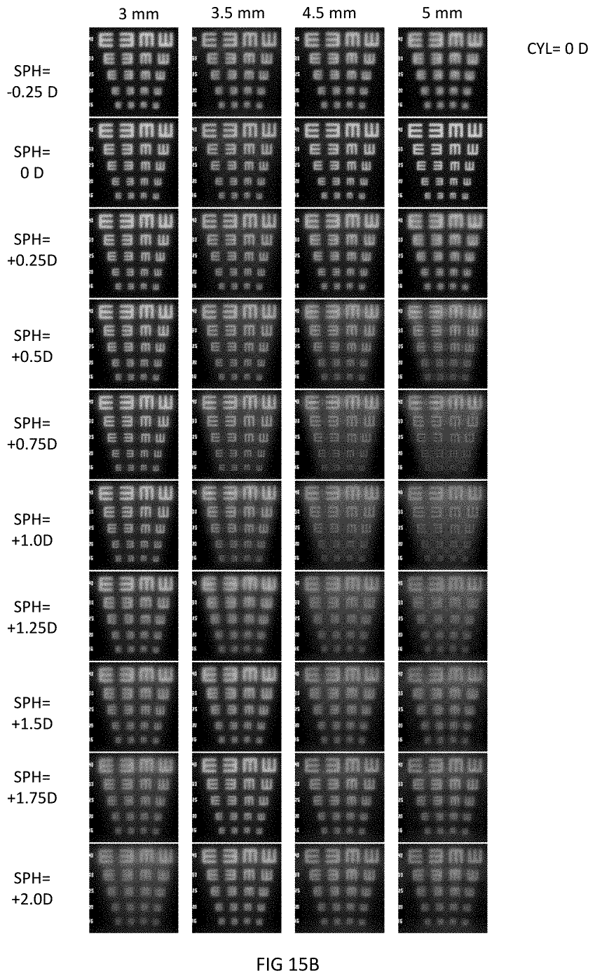

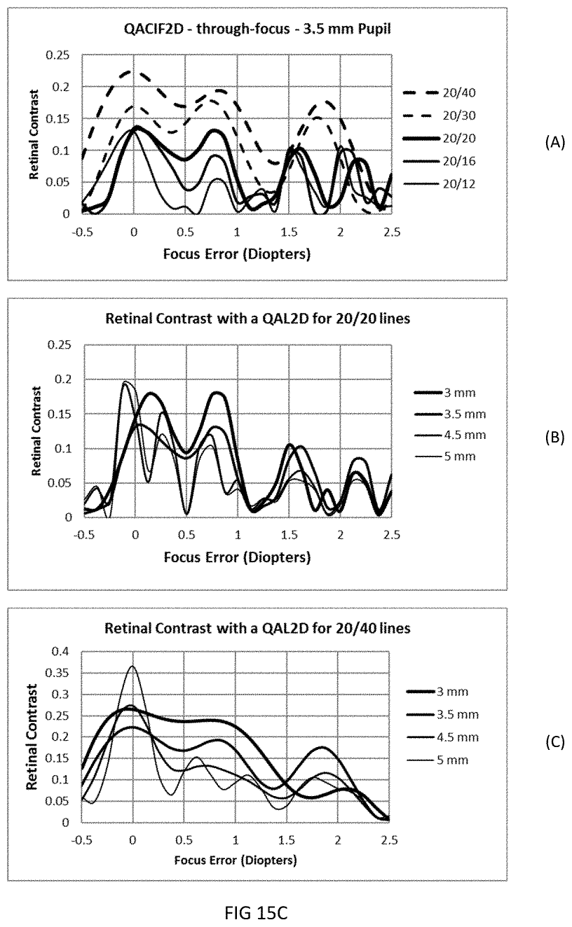

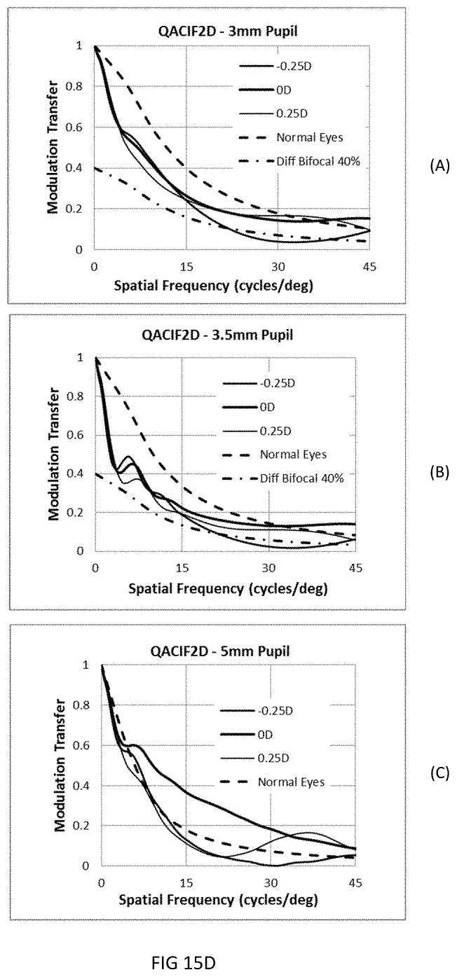

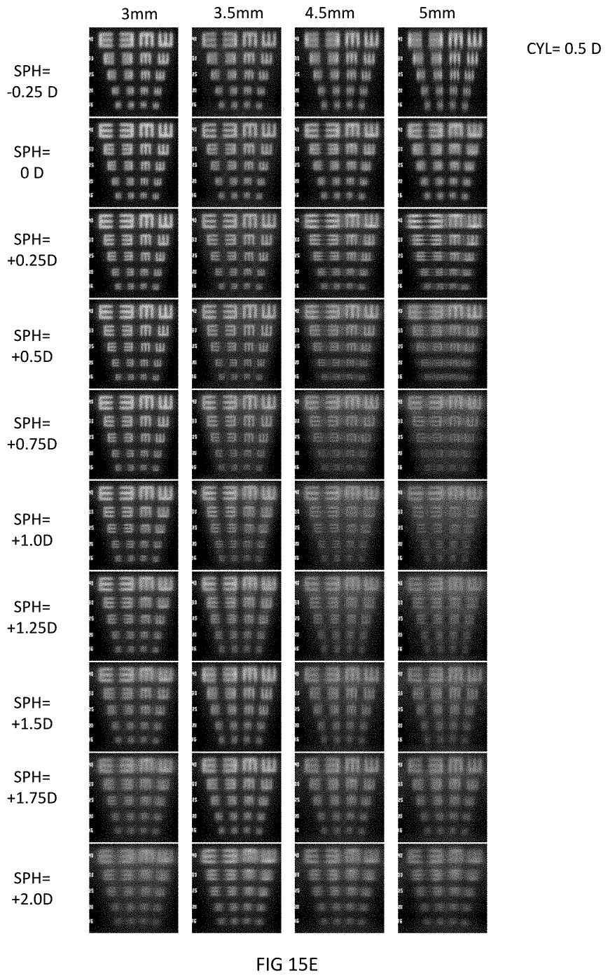

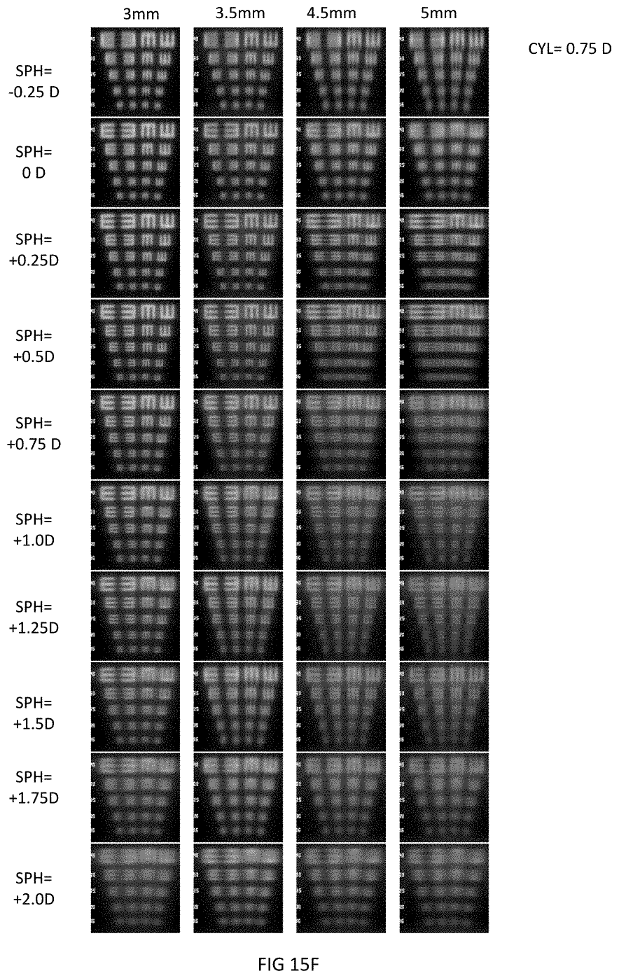

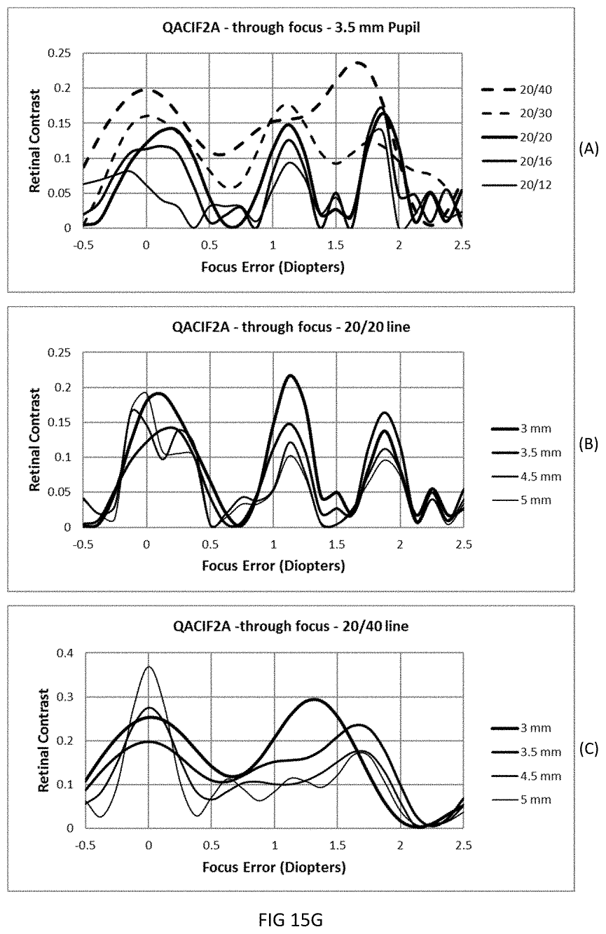

Performance of the wavefront QACIF2D is simulated and shown in FIG. 15A for the calculated Point Spread Functions (PSF) and in FIG. 15B for the calculated retinal images of an acuity chart. The parameter SPH is used to specify a focus error of the eye through focus. SPH=0 D specifies the best corrected vision at 4 meters. SPH=−0.25 D specifies the corrected vision at infinity. SPH=+2.0 D specifies a presbyopia correction of +2.0 D. We considered four pupil sizes of 3.0 mm and 3.5 mm for acuity tests, and 4.5 mm and 5.0 mm in diameter for night vision.

Form the calculated point-spread function in FIG. 15A between SPH=−0.25 D and SPH=+2.0 D, the lens provides three focus zones centered around 0D, +0.75 D, and last one around +1.75 D with twin peaks at +1.5 D and +2.0 D. For the pupil size of 3 mm and 3.5 mm in acuity tests, these foci are so close forming extended depth of focus that makes the lens nearly in focus throughout the focus range between SPH=−0.25 D and SPH=2.0 D, except for a relative weak focus point at SPH=+1.25 D.

FIG. 15C shows plots of calculated “through focus” retinal contrast of QACIF2D for a 3.5 mm pupil, and for 20/20 lines and 20/40 lines with pupil size between 3 mm to 5 mm. The QACIF lens can offer 20/20 or better vision for a first focus with extended depth of focus from −0.25 D to 1.0 D, and offer 20/20 or 20/25 between +1.50 D and +1.75 D. Visua acuity of 20/30 or better is expected through focus from −0.25 D to +2.0 D. These findings are can be confired in the caluclated retina image in FIG. 15B. Therefore, we see a nearly continously-in-focus lens with a slightly degraded vision at +1.25 D in all pupil sizes and +2.0 D for a 3 mm pupil.

FIG. 15D shows plots of calculated Modulation Transfer Function (MTF) of QACIF2D for far distances at infinity (−0.25 D), at 4 meters (0 D), and a focus error at +0.25 D for pupil sizes of 3 mm, 3.5 mm, and 5 mm. In FIG. 15D, we also show the mean MTFs of normal eyes labed as “Normal Eyes” as well as estimated MTFs of a diffractive bifocal lenses labled as “Diff Bifocal 40%”. Our QACIF2D will offer better contrast than diffractive multifocal lenses for far distances, and will have no contrast loss for spatial frequencies larger than 30 c/deg (20/20 or finer features) and a slight contrast loss for spatial freqencies less than 30 c/deg, when compared to normal human eyes. This is particularly true for real eyes because uncorrected astigmatism and coma in an eye can be mitigated by our QACIF2D lenses, and they will degrade quality of vision for conventional monofocal lenses and diffractive multifocal lenses.

We expect the QACIF2D lens can offer patient 20/16 or better acuity with relatively high contrast, and night vision for a pupil size of 4.5 mm and 5 mm will be exceptional.

FIG. 15E and FIG. 15F show calculated retinal images with a QACIF2D lens if the eye has uncorrected astigmatism of ½ D and ¾ D, respectively. It is clearly seen that images in FIG. 15E with an uncorrected CYL of 0.5 D is almost identical to those in FIG. 15B with CYL=0. Even for an uncorrected astigmatism of 0.75 D, shown in FIG. 15F, vision is still good between +0.25 D and +1.25 D.

In addition to de-astigmatism, QACIF2D is also pupil-size independent between 3 mm and 5 mm, which can be validated with retinal images in FIGS. 15A/15E/15F as well as through-focus plots (B) and (C) in FIG. 15c. This is completely different from conventional lenses shown in FIG. 5B and in FIG. 10B where optics with a large pupil are more sensitive to focus error and astigmatism.

Even without engaging any artificial accommodation of AIOLs, based on two fundamental features of the exemplary lens: 1) excellent acuity of 20/20 or 20/25 from SPH=−0.25 D to SPH=+2.0 D, 2) nearly independence of pupil sizes between 3 mm and 5 mm, we classify this type of lenses as Quasi-Accommodating and Continuously-in-Focus (QACIF) lenses for 2.0 D.

An ICL or phakic IOL with QACIF2D optics can treat everyone 45 years and older without cataract for myopia /hyperopia, astigmatism, and presbyopia, making all of them spectacle independent PLUS free from reading glasses.

FIG. 15G shows another design of Quasi-Accommodating and Continuously-in-Focus lens “QACIF2A”. It offers a pupil-size independent EDOF trifocal lens with a first focus with extended depth of focus between −0.25 D and +0.5 D, a second focus centered at +1.25 D, and a third focus at +1.75 D. QACIF2A can be used to complement to QACIF2D. If QACIF2A and QACIF2D are applied to two eyes separately, the patient can expect 20/20 or better vision for the entire focus range between −0.25 D and +2.0 D PLUS for all pupil sizes between 3 mm and 5 mm.

Two more designs of QACIF lenses are also listed in Table 5A. They share similar characteristics of nearly continuously-in-focus for a focus range of 2.0 D and high tolerance of uncorrected astigmatism.

In some embodiments, the wavefront Quasi Accommodating and Continuously-in-Focus (QACIF) Lens is configured as an implantable or wearable lens. The wavefront QACIF lens comprises: 1) a baseline Diopter power extending across an optical section of the lens for correction of far vision defects, and the optical section having a diameter between 5 mm and 8 mm and the correction of far vision defects including a focus error and/or a cylinder error, 2) a central aspherical section having a positive focus offset ϕ₁ and a positive spherical aberration S₁, the positive focus offset ϕ1 being less than 2.0 D and greater than 0.75 D, and the positive spherical aberration S₁ being larger than 0.25 microns and less than 2.75 microns in the central aspheric section having a diameter less than 2.75 mm and greater than 1.9 mm, 3) an annular aspherical section outside the central aspherical section inducing negative spherical aberration, and the annular aspherical section having an outer diameter less than 4.5 mm and greater than 2.5 mm. Positive spherical aberration for the QACIF lenses in the central aspherical section is calculated and listed for a diameter of 1.9 mm, 2.2 mm, and 2.75 mm in Table 5B.

The wavefront QACIF lens is configured as a contact lens, an Intraocular Lens (IOL), an Accommodating Intraocular Lens (AIOL), a phakic IOL, an ICL (Implantable Contact Lens or Implantable Collamer Lens), or a corneal inlay.

In one embodiment, the annular aspherical section outside the central aspherical section is further configured to have a positive focus offset larger than 0 and less than 1.5 D.

TABLE 5A
Exemplary designs of QACIF lenses in aspherical zones

| | Parameters | QACIF2A | QACIF2B | QACIF2C | QACIF2D |
|---|---|---|---|---|---|
| Central | Radius R₁ (mm) | 1.05 | 1.2 | 1.2 | 1.25 |
| Aspherical | S.A. S₁ Microns) | 0.85 | 0.8 | 0.8 | 1.0 |
| Zone | Focus offset Φ₁ | 1.55 | 1.15 | 1.25 | 1.25 |
| | (Diopter) | | | | |
| Annular | Radius R₂ (mm) | 1.75 | 1.75 | 1.75 | 1.75 |
| Aspherical | S.A. S₂ Microns) | −1.67 | −1.11 | −0.74 | −1.11 |
| Zone | Focus offset Φ₂ | 0.30 | 0.75 | 1.0 | 0.75 |
| | (Diopter) | | | | |

TABLE 5B
Positive spherical aberration of QACIF in the central aspherical zone

| | Diameter of central | | | | |
|---|---|---|---|---|---|
| | aspheric section | D₁ (mm) | 1.9 | 2.2 | 2.75 |
| QACIF2A | Spherical aberration in the central | S₁ (μm) | 0.57 | 1.02 | 2.50 |
| | section = 0.85* (D₁/2.1)⁴ | | | | |
| QACIF2B | Spherical aberration in the central | S₁ (μm) | 0.31 | 0.56 | 1.38 |
| | section = 0.80* (D₁/2.4)⁴ | | | | |
| QACIF2C | Spherical aberration in the central | S₁ (μm) | 0.31 | 0.56 | 1.38 |
| | section = 0.80* (D₀/2.4)⁴ | | | | |
| QACIF2D | Spherical aberration in the central | S₁ (μm) | 0.33 | 0.60 | 1.46 |
| | section = 1.0* (D₁/2.5)⁴ | | | | |

In another embodiment, the induced spherical aberrations in the aspherical sections are expressed in Optical Path Difference (OPD), or the wavefront errors across eye's pupil as

$$\begin{matrix}OPD ( ρ ) = & {S}_{1} * {( ρ \text{/} {r}_{0} )}^{4} & if ρ \text{<=} {r}_{0} \\ = & ( - {S}_{2} ) * {( ρ \text{/} {r}_{1} )}^{4} & if {r}_{0} < ρ \text{<=} {r}_{1}\end{matrix}$$

where ρ is a polar radius in the pupil plane, S₁ is positive and it measures the positive spherical aberration in the first zone having its peak value of S₁ at its boundary ρ=r₀, and r₀ is a radius of the first zone and is larger than 0.9 mm and less than 1.4 mm. (−S₂) is negative and it measures the negative spherical aberration in the second zone having its peak value of (−S₂) at its boundary ρ=r₁, and r₁ is the outer diameter of the second zone, is larger than 1.25 mm and less than 2.25 mm.

In yet another embodiment, the negative spherical aberration (−S₂) is more than 0.15 microns and less than 4.75 microns in magnitude for an outer diameter of the annular aspherical zone less than 4.5 mm and greater than 2.5 mm. Negative spherical aberration in the annular aspherical section is calculated for a diameter of 2.5 mm, 3.0 mm, and 3.75 mm and listed in Table 5C.

In still another embodiment, the aspherical sections further induce a generalized spherical aberration that is characterized as Optical Path Difference including terms of ρⁿ and n is an integer equal to or greater than 3.

In one embodiment, the wavefront QACIF lens is configured as a wavefront contact lens having a diameter between 9 mm and 16 mm, and the aspheric surface is either a front surface or a back surface of the contact lens. The back surface of the contact lens is further configured to have an aspheric shape at a lens periphery for preventing lens rotation on an eye if the contact lens is also a toric lens.

TABLE 5C
Negative spherical aberration in the annular aspheric section

| | Outer diameter of the annular | | | | |
|---|---|---|---|---|---|
| | aspherical section | D₂ (mm) | 2.5 | 3.0 | 4.5 |
| QACIF2A | Spherical aberration in the | S₁ (μm) | −0.43 | −0.90 | −4.56 |
| | annular section = −1.67* (D₂/3.5)⁴ | | | | |
| QACIF2B | Spherical aberration in the | S₁ (μm) | −0.29 | −0.6 | −3.03 |
| | annular section = −1.11* (D₂/3.5)⁴ | | | | |
| QACIF2C | Spherical aberration in the | S₁ (μm) | −0.19 | −0.4 | −2.02 |
| | annular section = −0.74* (D₂/3.5)⁴ | | | | |
| QACIF2D | Spherical aberration in the | S₁ (μm) | −0.29 | −0.6 | −3.03 |
| | annular section = −1.11 (D₂/3.5)⁴ | | | | |

In another embodiment, the wavefront QACIF lens is configured as a wavefront IOL, and it has optical section of about 6 mm, between 5 mm and 7 mm in diameter. The wavefront IOL has a front surface and a back surface, and at least one of the front and back surfaces is aspheric at the lens center.

In yet another embodiment, the QACIF IOL is further configured as an accommodating IOL.

In still another embodiment, the wavefront QACIF lens is configured as a wavefront ICL to be implanted between iris and natural lens of an eye, wherein the aspheric surface is a front surface or a back surface of the wavefront ICL lens.

In another embodiment, the QACIF ICL is achieved through a thickness variation in the optics if the baseline power is less than 1.0 D in magnitude.

In yet another embodiment, the wavefront QACIF lens is configured as a wavefront cornea inlay that can be implanted into cornea of the eye for vision correction, wherein the aspheric surface is a front surface or a back surface of the wavefront cornea inlay.

In another aspect, we disclose a wavefront Implantable Contact Lens (ICL) for an eye, and it comprises: a) a haptics section for fixing the ICL to an iris in an anterior chamber of an eye with an example in WO1999062434A1 or holding the ICL in place inside a posterior chamber of an eye with an example in U.S. Pat. No. 6,106,553, b) a wavefront lens that includes b1)a baseline Diopter power extending across an optical section with a diameter between 5 mm and 8 mm for a spherocylindrical correction, b2) a central section with a diameter between 1.65 mm and 2.5 mm that induces a positive spherical aberration plus a positive focus offset ϕ1 less than +3.0 D and greater than +0.5 D, b3) an annular section with an outer diameter less than 4.5 mm that induces a negative spherical aberration. The wavefront errors from the induced spherical aberrations and the focus offset in the central and annular sections creates one of 1) a quasi-accommodation and continuous-in focus lens, 2) a wavefront bifocal lens, 3) a wavefront trifocal lens.

In one embodiment, the wavefront ICL has a central aspherical section and an annular aspherical sections for inducing the required spherical aberrations.

In yet another aspect, we disclose a method of refractive correction for an eye, and it comprises the steps of: a) determining refractive errors of an eye for a far vision correction, and the refractive errors include at least a sphere power SPH, b) performing a refractive surgery that makes the post-op eye with an extended depth of focus from a first focus power ϕ₁ to a second focus power ϕ₂, and the sphere power SPH of the eye is targeted between ϕ₁ and ϕ₂ so that the post-op eye can retain excellent vision at far distances even if the eye has a post-op myopia progression between −0.5 D and −1.25 D. In one embodiment, the refractive surgery having an extended depth of focus involves in implanting a wavefront ICL with an extended depth of focus. For example, if an ICL with optics of QACIF2D is implanted into an eye with a targeted far distance at SPH=+0.75 D instead of SPH=0D, the eye will not only have a post-op 20/20 vision but also have excellent vision for a focus range from −0.25 D to +1.0 D, shown in FIG. 15B/15C. This is advantageous because 1) it can mitigate post-op myopic progression up to 1 D for young adults; 2) any post-op myopic progression less than 1 D will be beneficial starting from 40 years old when the post-op eye develops presbyopia.

### 5. Advantages of Wavefront Monofocal, Bifocal, Trifocal and QACIF Lenses

Conventional monofocal and diffractive multifocal lenses can be excellent based on optical designs and test results in labs, but their performance suffers from many issues when they are actually put into or onto a human eye.

The disclosed wavefront lenses (monofocal and multifical) solve everal fundamental problems of monofocal/multifocal lenses in the prior art: 1) eliminating halo and starbust associated with diffractive multifocal lenses, 2) eliminating blurred zone between foci of multifocal lenses, 3) improving quality of vision for patients by eliminating image distortion of conventional monofocal lenses and diffractive multifocal lenses, 4) improving chances of achieving best corrected vision of 20/20 by extending depth of focus for 20/20 plus increasing tolerence for uncorrected astigmatism, which has been shown in FIG. 9B/9G, in FIG. 12C, in FIG. 13C, in FIG. 14C, and in FIG. 15C.

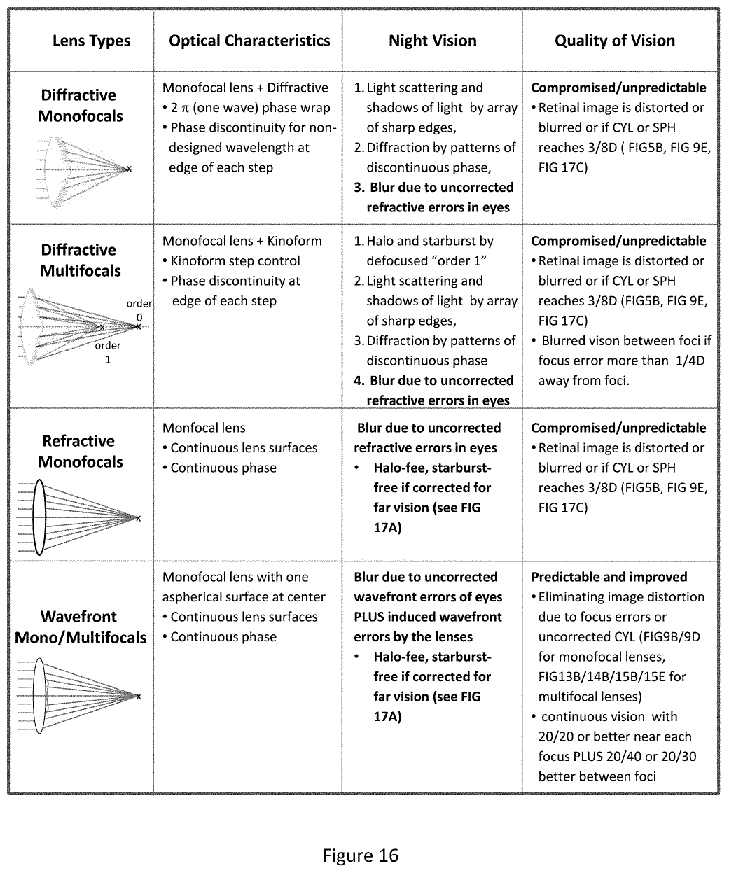

FIG. 16A provides a comparison of our wavefront mono/multifocal lenses of the present invention with conventional refractive monofocal lenses as well as difractive monofocal/multifocal lenses.

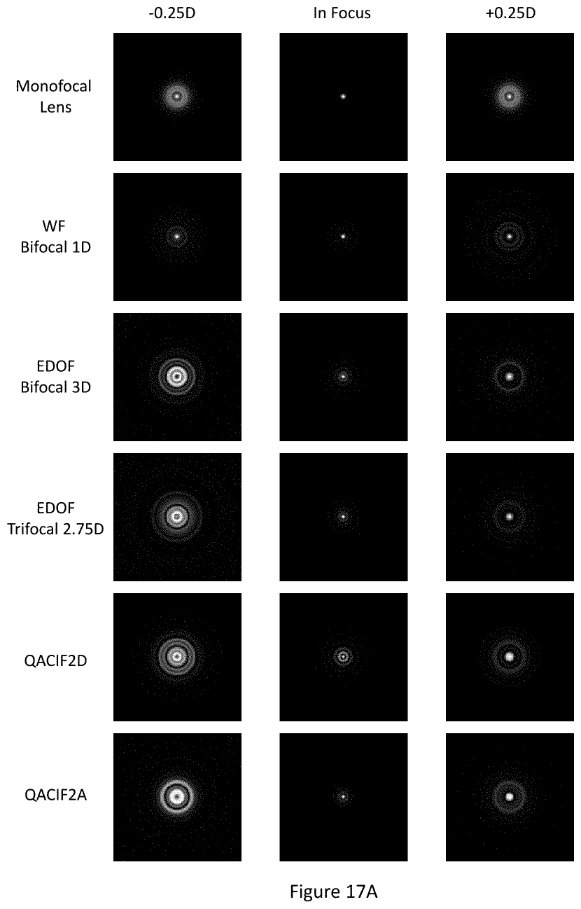

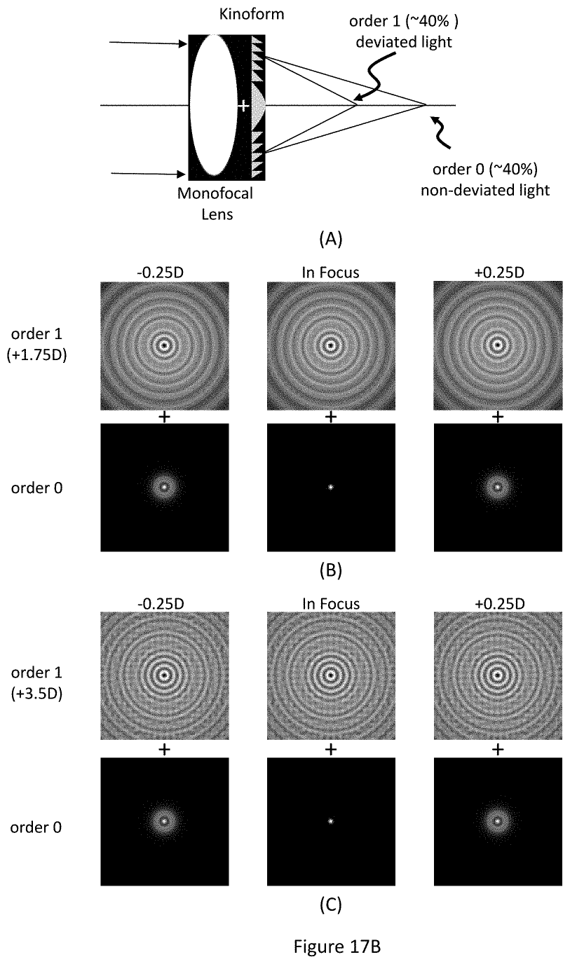

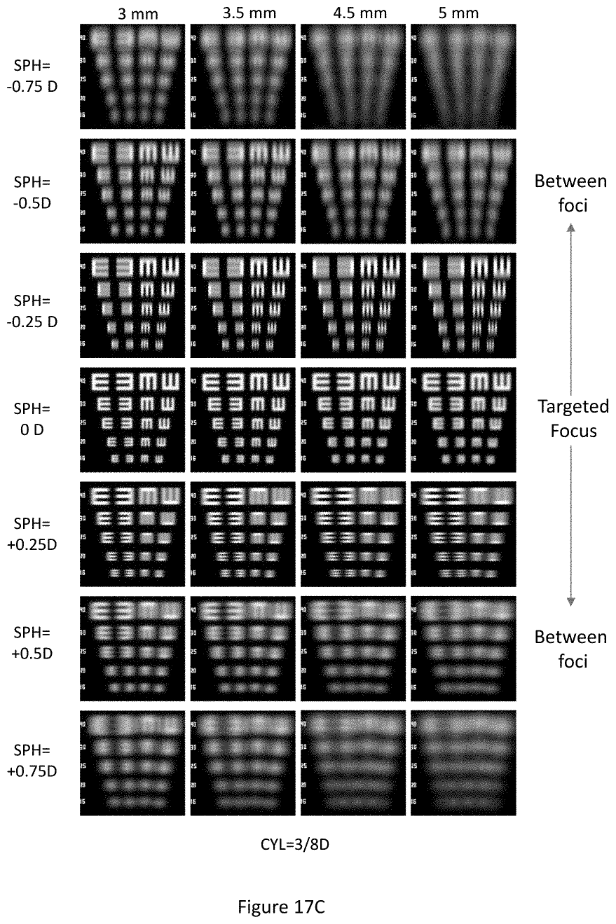

FIG. 17A shows calculated retinal images for pupil sizes of 5 mm at nighttime for a conventional refractive monofocal lenses in comparison with exemplary designs of wavefront multifocal lenses of the present inventions. We consider three focus settings: −0.25 D for far vision at infinity, 0 D for the targeted vision chart at 4 meters, +0.25 D for a presbyopia of +0.25 D. Angular dimension of each square in FIG. 16B is 0.25 degrees of arc. Compared to the sun in the sky in an angular size (about 0.5 degree of arc), the pattern of point-spread functions at the three far distances is very small: 1) about one 12^{th} for a conventional monofocal lens, and 2) one 14^{th} to one sixth for our wavefront EDOF bifocal, EDOF trifocal and QACIF lenses.

Diffractive multifocal lenses are constructed as a monofocal lens plus a Kinoform diffractive surface (see FIG. 17B in (A)). Retinal image of a diffractive multifocal lens consists of a non-deviated diffraction order “0” for the designed far vision correction, a deviated diffraction order “1” with an add-on power, and other diviated “higher” order diffraction images. Therefore, in addition to a focused image of diffraction order “0” that will be affected by wavefront errors of an eye, there is a defocused image of diffractive order “1” with a focus error of “the add-on power”, shown in FIG. 17B and (C) for an add-on power of +1.75 D and +3.5 D, respectively. Therefore, it is inevitable that halo and starburst will associate with diffractive multifocal lenses due to the defocued image of the near focus. In addition, nighttime symptoms with diffractive lenes can also be caused by 1) light scattering and shadows of light caused by a patterned of sharp edges, 2) diffraction pattern by discontinuous phase at each step in the Kinoform.

We can thus conclude that our wavefront multifocal lenses have similar night vision performance to that of a monofocal lens with a perfect correction for focus error. Nighttime halo and starburst of diffractive multifocal lenses are effectively eliminated. Additionally, our wavefront multifocal lenses would be better than conventional monofocal IOLs if the targeted far vision of a monofocal IOL is at around 1 meters for easing presbyopia instead of 4 meters for the best far vision.

Two other fundamental problems of conventional multifocal lense are 1) blurred vision between foci, 2) poor quality of vision associated with image distortion. We saw from calculated retinal images of a monofocal lens through focus in FIG. 10B that acceptable vision has a short depth of focus of about +/−0.25 D for a perfect correction of astigmatsim (CYL=0). If there is uncorrected astigmatism in the eye, however, focus depth will be further reduced. FIG. 17C shows calculated retinal images of a monofocal lens through focus between −0.75 D and +0.75 D with uncorrected astigmatism of ⅜ D. We can conclude: 1) retinal image distortion happens as soon as the focus error reches 0.25 D, 2) focus depth for 20/20 is much less than +/−0.25 D. For a diffractive bifocal IOL with 40% diffraction efficiency for far distances, the retinal images are similar as those in FIG. 10B with CYL=0 and FIG. 17C with CYL=⅜ D but with a contrast reduction of (1-40%) across all spatial freqencies. Therefore, for a multifocal lens with an add-on power larger than 1.5 D, we will expect blurred vision or distorted vision between foci for any focus distance with a focus error about 0.25 D from either of the foci.

Completely blurred vision and distored vision between foci is effectively resolved with our wavefront bifocal/ trifocal and QACIF lenses, shown in FIG. 9B/9D/9G, FIG. 15B/15E, FIG. 12B, FIG. 13B, FIG. 14B. Our wavefront lenses for presbyopia provide continous vision with 20/40 or better acuity throughout the focus range in each design.

### 6. Liquid Ophthalmic Lenses

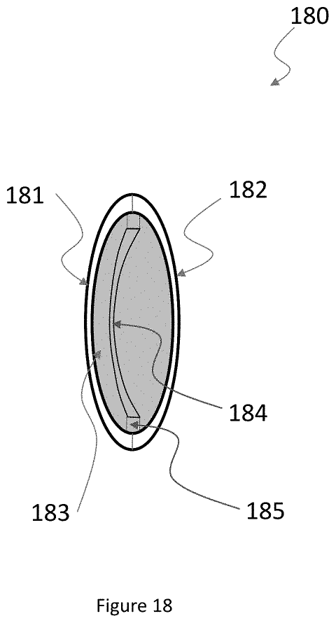

In one aspect of the present invention, we disclose a liquid ophthalmic lens (180) in FIG. 18. It comprises: 1) a liquid lens portion having a flexible bag formed by a front optical element (181) and a back optical element (182) and liquid (183) filled in the flexible bag formed by the front and the back optical elements, 2) a solid optical element (184) immersed in the liquid of the liquid lens section, configured to alter the refractive properties of the liquid lens, 3) a mounting mechanism (185) to fix the solid optical element (184) to the flexible bag.

In one embodiment, the liquid lens portion is configured to be deformable between an unaccommodated state for a nominal refractive power and an accommodated state for a different refractive power. The solid optical element (184) has a front surface and a back surface and an index of refraction n₁, which is different from that of the liquid (n₂).

Many mechanisms for attaching a liquid lens to a surgical eye are in the prior art for accommodation control of the liquid lens. In one embodiment, the liquid ophthalmic lens further comprises a haptic portion configured to deform in response to forces applied by movement of ciliary muscles of an eye, the haptic portion having an interior liquid volume in fluid communication with the liquid lens portion.

In yet another embodiment, the solid optical element immersed in the liquid lens portion is optically a spherical lens configured to change the spherical power of the combined liquid lens. This design makes it suitable for a large population with different IOL power requirements using the same structures for the front and back element of the liquid lens. The liquid lens has an IOL power of 29 D without the immersed solid optical element, with one structure design for its front surface (101), back surface (102), and the liquid. Its shape can be deformed to achieve a fixed range of accommodation up to 4.0 D. If the immersed solid optical element can be selected for one optical power between +11.0 D and −11.0 D, the same structure of liquid lens plus the immersed lens will achieve a focus range between +18 D and +40 D. One advantage of using one structure for the deformable liquid lens is to reduce potential variations in accommodation control due to different structures of deformable liquid lenses.

In yet another embodiment, the immersed solid optical element in the liquid lens portion is optically a toric lens configured to add a cylinder power to the liquid lens. This makes it suitable for accommodating toric IOLs to use the same structure of accommodating IOLs for its front and back element of the liquid lens.

In still another embodiment, the solid optical element immersed in the liquid lens portion induces spherical aberration(s) and a focus offset(s) in the center section of the liquid lens with a diameter around 3.5 mm, e.g., between 2.2 mm and 4.5 mm, and the induced spherical aberration(s) and focus offset(s) provides mitigation to uncorrected astigmatism, coma, focus errors, presbyopia left by the liquid IOL when it is implanted into a human eye.

### 7. Wavefront Corneal Implants for Presbyopia Corrections

In one aspect, we disclose a wavefront corneal implant that is configured for a presbyopia correction for an eye. The wavefront corneal implant comprises an optical element having a diameter D₁ between 2.0 mm and 4.5 mm. The optical element has a base section of uniform thickness, and an add-on section for refractive corrections. The overall thickness is between 10 microns and 50 microns. The add-on section induces wavefront errors into an eye that include: 1) a positive focus power ϕ₁ between 1.0 D and 2.5 D at the center section having a diameter D₀ of 1.5 mm to 2.5 mm, 2) a positive spherical aberration in the center section, 3) a negative spherical aberration in an annular section outside of the center section.

In one embodiment, the annular section can further induce a focus error between −1.0 D and +1.0 D.

Differing from the conventional corneal inlays in the form of a positive lens in U.S. Pat. No. 8,057,541 B2, #8,900,296B, the wavefront inlay using one of the wavefront bifocal, wavefront trifocal, and QACIF designs offers excellent acuity of 20/20 or better for far distances and 20/20 or better for near vision with an add-on power between +1.0 D and +2.5 D.

The base section of uniform thickness can be configured as a parallel plate or to have a curvature radius of about 7.8 mm, like the curvature radius of a normal cornea. In one embodiment, the add-on section is configured to vary in thickness across the corneal implant only.

In another embodiment, the corneal implant is made of a biocompatible material, and is made through a process of molding or lathing.

In another embodiment, the corneal implant is made of human cornea tissue from donors, and is made through a process of laser ablation using UV light and/or using laser cutting with short pulse lasers.

In yet another embodiment, the add-on optical section of the corneal implant comprises a thickness variation as well as a change of refractive index. The change of refractive index can be achieved using a short pulse laser. Employing a change of refractive index in the corneal implant has an advantage in that it allows fine tuning of the wavefront map because a change of refractive index is very small, in the range between 0.001 and 0.03.

In still another embodiment, the wavefront corneal implant is made of human cornea tissue from a donor in a process of laser ablation/cutting as well as index change of the corneal tissue using a short pulse laser.

In one embodiment, the add-on section further includes a baseline Diopter power extending across the corneal implant for 1) a conventional spherical correction or 2) a sphero-cylindrical correction for far vision defects.

In another embodiment, the add-on section of the corneal implant further induces a generalized spherical aberration that is characterized as wavefront errors in term of ρⁿ, and n is an integer equal to or greater than 3.

### 8. Wavefront Surgical Procedures for Presbyopia Corrections of Human Eyes

In one aspect of the present invention, we disclose a wavefront method of surgical procedure for presbyopia corrections of human eyes. The wavefront procedure comprises: 1) using a first laser beam to generate a central island in a central pupil having a diameter D₁ between 2.0 mm and 4.5 mm, an optical effect of the central island being represented by a wavefront error W₁(r); 2) using a second laser beam to change the refractive index of corneal tissue by δn and a depth distribution d(r) of tissue with index change in the central pupil. A combination effect W₁(r) of the central island due to the first laser and a Gradient-Index (GRIN) optics created through the laser writing using a second laser beam in the cornea causes combined wavefront errors that include: a) a positive focus power ϕ₀ at the center section having a diameter D₀ of 1.5 mm to 2.5 mm, and the positive focus power being between 1.0 D and 2.50 D; b) a positive spherical aberration in the center section, c) a negative spherical aberration in an annular section, outside of the center section, d) a focus error between −1.0 D and +1.0 D in the annular section.

In one embodiment, the wavefront procedure further includes using the first laser to generate a baseline refraction correction for a conversional spherical correction or a spherocylindrical correction for far vision defects when necessary, and the baseline refractive correction is either performed by tissue ablation using a UV beam or by tissue removal using a short pulse laser.

### 9. Wavefront Lenses for Contact Lens Fitting

In one aspect of invention, we disclose a wavefront contact lens for testing human eyes. The contact test lens comprises: 1) a hypothetical baseline Diopter power extending across an optical section, which has a diameter between 5 mm and 9 mm, and the hypothetical baseline Diopter power being theoretical and not for a specific eye, b) a central aspherical section at least in a center of the lens having a diameter between 2.2 mm and 4.5 mm that uses at least one aspheric surface to induce additional spherical aberration at central pupil of the eye.

In some embodiments, the baseline hypothetical Diopter power includes at least one of the following: a) optically plano that has no refractive power, b) a correction for eye's astigmatism, c) a hypothetical spherocylindrical correction.

In one embodiment, the test contact lens further includes a focus offset in the central aspherical section.

In another embodiment, the central aspherical section is configured to have at least one aspheric surface for inducing a positive spherical aberration in a first zone and a negative spherical aberration in a second zone, wherein the first zone and the second zone are concentric.

In another aspect, we disclose a method for prescribing contact lenses. The method comprises the steps of: 1) determining a spherocylindrical correction for a contact lens that includes SPH for a spherical power, and/or astigmatism specified by CYL and AXIS, 2) placing a wavefront contact lens onto a tested eye, and the test contact lens comprising: 2a) a hypothetical baseline Diopter power extending across an optical section and having a diameter of 5 to 9 mm, 2b) a central aspherical section at least in a center of the lens having a diameter D₀ between 2.2 mm and 4.5 mm that uses at least one aspheric surface to induce additional spherical aberration at central pupil of the eye, 3) updating the determined spherocylindrical correction for a contact lens subjectively using a phoropter, 4) prescribing a contact lens based on the updated spherocylindrical correction and the optical properties of the wavefront contact lens placed onto the tested eye.

In yet another aspect, we describe a system for prescribing contact lenses. The system comprises: 1) a wavefront module that measures aberrations in an eye, 2) a processor module for 2a) determining a spherocylindrical correction for a contact lens, and the spherocylindrical correction consisting of a focus error SPH and/or astigmatism specified by CYL and AXIS, and 2b) determining at least an aspherical component in the central part of the lens having a diameter between 2.2 mm and 4.5 mm, and the aspherical component of the lens inducing spherical aberration into the corrected eye for mitigating the estimated residual refractive errors of the eye under a conventional spherocylindrical correction, 3) a phoropter module for updating the determined spherocylindrical correction for a contact lens subjectively by keeping or modifying at least the spherical power SPH, 4) an output module for prescribing a contact lens based on updated spherocylindrical correction and the aspherical component in the central part of the lens.

In one embodiment, the estimated residual refractive errors of the eye under a conventional spherocylindrical correction include the following: astigmatism, coma, focus error, and presbyopia.

In another embodiment, updating the determined spherocylindrical correction for a contact lens subjectively further includes placing a wavefront contact lens onto a tested eye, and the wavefront contact lens contain at least an aspherical component in the central part of the lens having a diameter between 2.2 mm and 4.5 mm, and the aspherical component of the lens induces spherical aberration into the corrected eye. The system can further provide a selection between a conventional contact lens and a wavefront contact lens.

In still another embodiment, determining at least an aspherical component in the central part of the lens for vision optimization for the purpose of 1) increasing contrast in the Modulation Transfer Function at high spatial frequency higher than 30 cycles/deg and improving the best corrected acuity beyond 20/20, 2) eliminating image distortion, particularly for eliminating phase reversal in the Phase Transfer Function (PTF) at low spatial frequencies below 30 cycles/deg.

### 10. Therapeutic Treatments for Eye's High-Order Aberrations

Inducing spherical aberration in the central pupil of the eye for vision correction is powerful, and provides mitigation of uncorrected astigmatism, focus error, coma, and presbyopia. Our wavefront engineered lenses will be also effective for improving therapeutic correction of the eye's high-order aberrations.

In one aspect, we disclose a contact lens for therapeutic treatment of an eye, comprising: a) a baseline wavefront refractive correction extending across an optical section of the lens for correction of far vision defects, the optical section having a diameter between 5 mm and 8 mm, and the baseline wavefront refractive correction includes a focus error, astigmatism, and high-order Zernike aberrations such as coma, spherical aberration, b) at least an aspherical section at the lens center inducing spherical aberration(s) into eye's central pupil for mitigating imperfections in the correction of far vision defects.

The imperfection in the correction of far vision defects in one embodiment includes one or more of the following deficiencies: 1) registration errors between the baseline wavefront correction and the wavefront errors in the eye, 2) limitations in correcting some aberrations in the baseline wavefront refractive correction, and 3) imperfection in measuring the baseline wavefront correction for far vision defects.

In one embodiment, the therapeutic contact lens further includes an outer section that has a diameter between 6.0 and 13 mm, and is optically transparent.

In another embodiment, the therapeutic contact lens is configured as an EDOF monofocal, EDOF bifocal, EDOF trifocal, and QACIF lens.

### 11. Methods and Devices for Improving Vision Devices Containing Eyes

Inducing spherical aberration in the central pupil of eye for vision correction has been found powerful in correcting uncorrected astigmatism, coma, focus error, and presbyopia left by conventional correction lenses. It can also be applied to improve a vision device that contains an eye as an image sensor.

In one aspect of the invention, we disclose an improved vision device that uses a human eye as an image sensor. The vision device comprises 1) an optical image module, 2) an eyepiece module being the lens or a group of lenses that is closest to the eye. Either the eyepiece or the optical image module induces spherical aberration at least into the human eye in a central pupil having a diameter D₀ between 2.2 mm and 4.5 mm.

In one embodiment, the vision device is one of the followings: a Virtual Reality (VR) device, a microscope including a stereo microscope and a surgical microscope, a telescope including a monocular or a binocular, a vision goggle including a night vision goggle and a game goggle.

In another embodiment, the optical image module provides one of the followings: a) a microscopic view of objects nearby; b) a telescopic view of distant objects; c) a view of an electronic display.

In yet another embodiment, the eyepiece has a central aspherical section inducing spherical aberration within a small numerical aperture near the optical axis and cover diameter of eye's pupil up to 4.5 mm.

In still another embodiment, the central aspherical section of the eyepiece further incudes a focus offset beyond the induced spherical aberration.

In one embodiment, the eyepiece has aspherical sections in the center for inducing wavefront errors including: a) a positive focus power between +1.0 D and +2.5 D at a center section having a diameter D₀ of 1.5 mm to 2.5 mm; b) additional positive spherical aberration in the center section; c) a negative spherical aberration in an annular section with an outer diameter between 2.5 mm and 4.5 mm outside of the center section.

In still another embodiment, the eyepiece further corrects spherical aberration of human eyes at pupil periphery if the vision device uses the eye's pupil beyond 4.5 mm in diameter.

In one embodiment, the eyepiece induces spherical aberrations of opposite signs into an observer's eye at least in a central pupil having a diameter D₀ between 3.0 mm and 4.5 mm.

In another embodiment, inducing spherical aberration at least into an observer's eye in a central pupil is achieved by an addition of a phase plate to a conventional eyepiece. The eyepiece can further provide focus adjustment for eyes with different amounts of myopia or hyperopia, and a pupil tracking device, which assists the alignment of the optical axis of the eyepiece to the pupil center of the eye.

In still another embodiment, the vision device is further integrated with a surgical instrument or a head-mount device.

In another aspect of the present invention, we disclose an eyepiece, being the lens or group of lenses that is closest to the eye, and it comprises one aspheric surface to induce spherical aberration at least in the central zone of the optics having a diameter D between 2.2 mm and 4.5 mm. In one embodiment, the eyepiece further corrects spherical aberration of human eyes at pupil periphery if the vision device uses the eye's pupil beyond 4.5 mm in diameter.

Ever since its discovery in the 19th century, spherical aberration has been considered an optical defect that causes image blur like astigmatism, coma. In the present invention, however, we have shown, just like some harmful materials and agents used in drugs for treating diseases when they are delivered into human bodies with a small enough amount in a controlled manner to be efficacious, that spherical aberration may intentionally be delivered into the central pupil of an eye with a lens in a controlled manner for treatment of common refractive errors left uncorrected by ophthalmic lenses, including astigmatism, coma, focus errors, and presbyopia. These uncorrected refractive errors degrade quality of vision corrections for almost every eye, leading to poor acuity, distorted vision, and nighttime symptoms.

When these lenses with induced spherical aberration(s) are placed into or onto an eye, a lens decentration from the visual axis of an eye is possible. We have simulated optical quality with lens decentration, and concluded that a lens decentration within 0.5 mm has no or negligible impact on performance of the lens.

We must also point out that an excess amount of spherical aberration at the eye's pupil periphery can still degrade night vision. Spherical aberration at the pupil periphery can be treated just like conventional aspherical lenses. The wavefront lenses (monofocals, bifocals, trifocals, QACIF lenses) have several options for their optical properties at the pupil periphery beyond their central aspherical sections. These wavefront lenses can be configured to include: 1) a spherical section outside the central aspheric section, 2) a toric shape throughout a toric lens, 3) an aspherical section outside the central aspheric section for modifying spherical aberration in the correction lens with a high refractive power or/and for correcting a mean spherical aberration in normal eyes at the pupil periphery.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only and is not intended to limit the invention.

What is claimed is:

1. A non-diffractive multifocal lens for an eye, comprising:
an optic having an anterior surface and a posterior surface, said optic including an optical section having a diameter, D₂, where D₂ is equal to or less than 8 mm, wherein the optical section is comprised of a plurality of optical sub-sections, further wherein:
I) in a central optical sub-section having a diameter, D₁, where D₁ is between 2.5 mm and 4.5 mm, the central optical sub-section is configured as a non-diffractive multifocal lens with a plurality of foci, characterized by
(a) a first focus with a refractive power Φ₁ and the focus in itself has an Extended Depth of Focus (EDOF) compared to a monofocal lens having the same diameter, D₁, wherein the first focus is configured for the correction of an eye's myopia or hyperopia effects and
(b) at least another focus, Φ₂, having a more positive refractive power than Φ₁, wherein Φ₂=Φ₁+|δΦ|, where |δΦ| is between 0.5 D and 3.5 D for a presbyopia correction;
II) in an outer annular optical sub-section having an outer diameter D₂, the optic is configured as a monofocal lens with a refractive power of Φ₀ sufficient to provide a baseline correction for a far vision defect of a myopia or a hyperopia of the eye;
III) the refractive power Φ₁ of the first focus in the central optical sub-section is configured approximately equal to Φ₀ within 0.4 Diopter;
further wherein the optical section is characterized by
I) having a baseline Diopter power, Φ₀, extending across the optical section with a diameter D₂,
II) inducing a positive spherical aberration in a first zone and a negative spherical aberration in a second zone, wherein the first zone and the second zone are concentric and form the central optical sub-section with a diameter D₁,
III) having a positive focus offset δΦ₀ at an inner central sub-section having a diameter, D₀, smaller than D₁, wherein the positive focus offset is more than +0.5 Diopters.

2. The lens of claim 1, wherein the diameter, D1, of central optical sub-section is 3 mm.

3. The lens of claim 1, wherein the Extended Depth of Focus (EDOF) in the first focus Φ₁, compared to a monofocal lens at the same diameter D₁, can be quantified by its through-focus point-spread function or its through-focus contrast for the spatial frequencies of 30 cycles/deg, wherein 30 cycles/deg relate to an acuity metric of 20/20.

4. The lens of claim 1, wherein a contrast metric of the first focus (Φ₁) is equal to or higher than a threshold value of 10% for spatial frequency of 30 cycles/deg.

5. The lens of claim 1, wherein a maximum contrast metric of the first focus (Φ₁) for far vision is equal to or greater than that of the other focus (Φ₂=Φ1+|δΦ|) for a presbyopia correction at spatial frequency of 30 cycles/deg.

6. The lens of claim 1, wherein the central optical sub-section has at least one aspherical surface.

7. The lens of claim 1, wherein inducing a positive spherical aberration in the first zone and a negative spherical aberration in the second zone comprises Gradient-Index (GRIN) optics.

8. The lens of claim 1, further including a haptics section outside the optic, configured as one of an Intraocular Lens (IOL), a phakic IOL, an Implantable Contact Lens (ICL), an Accommodating Intraocular Lens (AIOL).

9. The lens of claim 1, further including a non-refractive section outside the optic, configured as contact lens.

10. The lens of claim 1, wherein the induced positive spherical aberration in a first zone and a negative spherical aberration in a second zone is expressed in Optical Path Difference (OPD) or wavefront errors as
$$\begin{matrix}OPD ( ρ ) = & {S}_{1} * {( ρ \text{/} {r}_{0} )}^{4} & if ρ \text{<=} {r}_{0} \\ = & ( - {S}_{2} ) * {( ρ \text{/} {r}_{1} )}^{4} & if {r}_{0} < ρ \text{<=} {r}_{1}\end{matrix}$$
wherein ρ is a polar radius, S₁ is positive and represents the positive spherical aberration in a first zone while r₀ is the radius of the first zone, less than 1.2 mm and more than 0.9 mm, and wherein (−S₂) is negative and represents the negative spherical aberration in the second zone while r₁ is the outer radius, less than 2.25 mm and more than 1.25 mm.

11. The lens of claims 10, wherein the lens in the central optical sub-section further induces a generalized form of spherical aberration that is characterized as the summation of a plurality of terms of ρⁿ, wherein n is an integer equal to or greater than three (3).

12. The lens of claim 1, further configured as a toric lens.

13. The lens of claim 1, further configured to include an aspherical surface for the outer annular optical sub-section in order to modify spherical aberration at the pupil periphery in human eyes, including but not limited to the correction of an averaged spherical aberration in normal population.

14. A non-diffractive trifocal lens for an eye, comprising:
an optic having an anterior surface and a posterior surface; said optic includes an optical section having a diameter D₂ equal to or less than 8 mm and configured into a plurality of optical sub-sections, further wherein:
I) in a central optical sub-section having a diameter D₁ between 2.5 mm and 4.5 mm, the central optical sub-section is configured as a non-diffractive trifocal lens, characterized by having a first refractive power Φ₁ for correction of myopia or hyperopia and two additional foci Φ₂ and Φ₃, wherein Φ₂=Φ₁+|δΦ₁| and Φ₃=Φ₁+|δΦ₂| respectively, and |δΦ₁| and |δΦ₂| are between 0.5 D and 3.5 D for a presbyopia correction;
II) in an outer annular optical sub-section having an outer diameter D₂, the optic is configured as a monofocal lens with a refractive power of Φ₀ sufficient to provide a baseline correction for a far vision defect of a myopia or a hyperopia of eye;
III) the refractive power Φ₁ for the first focus in the central optical sub-section is configured approximately equal to Φ₀ in the outer annular sub-section within 0.4 Diopters;
further wherein the optical section is characterized by
I) having a baseline Diopter power, Φ₀, extending across the optical section with a diameter D₂,
II) inducing a positive spherical aberration in a first zone and a negative spherical aberration in a second zone, wherein the first and the second zones are concentric and form the central optical sub-section with a diameter D₁,
III) having a positive focus offset δΦ₀ at an inner central sub-section having a diameter, D₀, smaller than D₁, wherein the positive focus offset is less than +3.0 Diopters.

15. The lens of claim 14, wherein the diameter, D1, of the central optical sub-section is 3 mm.

16. The lens of claim 14, wherein a contrast metric of the first focus (Φ₁) for the far vision correction is equal to or higher than a threshold value of 10% for spatial frequency of 30 cycles/deg, relating to an acuity metric of 20/20.

17. The lens of claim 14, wherein a maximum contrast metric of the first focus (Φ₁) in the central optical sub-section for far vision is configured equal to or higher than those of the other two foci for a presbyopia correction at spatial frequency of 30 cycles/deg.

18. The lens of claim 14, wherein the three foci of the trifocal lens in an central optical sub-section are sufficiently separated and all three foci have contrast approximately equal to or higher than a threshold value around 10% for spatial frequency 30 cycles/deg, relating to an acuity metric of 20/20.

19. The lens of claim 14, wherein the central optical sub-section has at least one aspherical surface.

20. The lens of claim 14, wherein inducing a positive spherical aberration in the first zone and a negative spherical aberration in the second zone comprises Gradient-Index (GRIN) optics.

21. The lens of claim 14, further including a haptics section outside the optic and is configured as an Intraocular Lens (IOL), a phakic IOL or an Implantable Contact Lens (ICL), an Accommodating Intraocular Lens (AIOL).

22. The lens of claim 14, further including a non-refractive section outside the optic and is configured a contact lens.

23. The lens of claim 14, wherein the induced positive spherical aberration in a first zone and a negative spherical aberration in a second zone is expressed in Optical Path Difference (OPD) or wavefront errors as
$$\begin{matrix}OPD ( ρ ) = & {S}_{1} * {( ρ \text{/} {r}_{0} )}^{4} & if ρ \text{<=} {r}_{0} \\ = & ( - {S}_{2} ) * {( ρ \text{/} {r}_{1} )}^{4} & if {r}_{0} < ρ \text{<=} {r}_{1}\end{matrix}$$
wherein ρ is a polar radius, S₁ is positive and represents the positive spherical aberration in a first zone while r₀ is the radius of the first zone, less than 1.2 mm and more than 0.9 mm, and wherein (−S₂) is negative and represents the negative spherical aberration in the second zone while r₁ is the outer radius, less than 2.25 mm and more than 1.25 mm.

24. The lens of claims 23, wherein the lens in the central optical sub-section further induces a generalized form of spherical aberration that is characterized as the summation of a plurality of terms of ρⁿ, wherein n is an integer equal to or greater than three.

25. The lens of claim 14, further configured as a toric lens.

26. The lens of claim 14, further configured to include an aspherical surface for the outer annular optical sub-section in order to modify spherical aberration at the pupil periphery in human eyes, including but not limited to the correction of an averaged spherical aberration in normal population.

27. A quasi-accommodation lens for an eye, comprising:
an optical section up to 8 mm in diameter (D₂) including a non-diffractive multifocal structure in a central optical sub-section with a diameter (D₁) between 2.5 mm and 4.5 mm, wherein the non-diffractive multifocal structure provides a substantially continuous and uninterrupted vision for a focus range larger than 1.0 D and less than 3.5 D, further characterized by
I) having a plurality of foci with their contrast larger than a threshold value of 8% to 10% for spatial frequency of 30 cycles/deg (equivalent to 100 lp/mm) so that 20/20 acuity can be achieved in a first focus for far vision at distance and in at least another focus for a presbyopia correction,
II) having a minimum contrast of 6% to 8% throughout the focus range for spatial frequency of 15 cycles/deg (equivalent to 50 lp/mm) so that 20/40 acuity can always be achieved for continuous and uninterrupted vision; wherein the non-diffractive multifocal structure is achieved by inducing spherical aberration(s) into the central optical section.

28. The lens of claim 1, wherein the diameter, D1, of central optical sub-section is 3 mm.

29. The lens of claim 27, wherein refractive property of the lens is further characterized by I) having a baseline Diopter power Φ₀ extending across the optical section with a diameter D₂, II) inducing a positive spherical aberration in a first zone and a negative spherical aberration in a second zone, wherein the first zone and the second zone are concentric and they form the central optical sub-section with a diameter D₁, III) having a positive focus offset |δΦ₀| at an inner central optical sub-section with a diameter (D₀) smaller than D₁.

30. The lens of claim 29, wherein the induced positive spherical aberration in a first zone and a negative spherical aberration in a second zone is expressed in Optical Path Difference (OPD) or wavefront errors as
$$\begin{matrix}OPD ( ρ ) = & {S}_{1} * {( ρ \text{/} {r}_{0} )}^{4} & if ρ \text{<=} {r}_{0} \\ = & ( - {S}_{2} ) * {( ρ \text{/} {r}_{1} )}^{4} & if {r}_{0} < ρ \text{<=} {r}_{1}\end{matrix}$$
wherein ρ is a polar radius, S₁ is positive and represents the positive spherical aberration in the first zone while r₀ is the radius of the first zone, less than 1.2 mm and more than 0.9 mm, and wherein (−S₂) is negative and represents the negative spherical aberration in the second zone while r₁ is the outer radius, less than 2.25 mm and more than 1.25 mm.

31. The lens of claims 29, wherein the lens in the central optical sub-section further induces a generalized form of spherical aberration that is characterized as the summation of a plurality of terms of ρⁿ, wherein n is an integer equal to or greater than there (3).

32. The lens of claim 27, wherein the central optical sub-section has at least one aspherical surface.

33. The lens of claim 29, wherein inducing a positive spherical aberration in the first zone and a negative spherical aberration in the second zone comprises Gradient-Index (GRIN) optics.

34. The lens of claim 27 further includes a haptics section outside the optic and is configured as an Intraocular Lens (IOL), a phakic IOL or an Implantable Contact Lens (ICL), an Accommodating Intraocular Lens (AIOL).

35. The lens of claim 27 further includes a non-refractive section outside the optic and is configured a contact lens.

36. The lens of claim 27, further configured as a toric lens.

37. The lens of claim 27, further configured to include an aspherical surface for the outer annular optical sub-section in order to modify spherical aberration at the pupil periphery in human eyes, including but not limited to the correction of an averaged spherical aberration in normal population.
